(19) United States
(12) Patent Application Publication
(10) Pub. No.: US 2010/0210025 A1
(43) Pub. Date: Aug. 19, 2010

(54) Common Module Profiling of Genes

(75) Inventors: Merridee WOUTERS, Lindfield (AU); Richard George, Darlinghurst (AU)

Correspondence Address: MORGAN, LEWIS & BOCKIUS LLP (SF), One Market, Spear Street Tower, Suite 2800, San Francisco, CA, 94105, US

(73) Assignee: Victor Chang Cardiac Research Institute limited, Darlinghurst (AU)

(21) Appl. No.: 12/709,292

(22) Filed: Feb. 19, 2010

Related U.S. Application Data
(63) Continuation-in-part of application No. 11/504,914, filed on Aug. 15, 2006, now abandoned.

(51) Int. Cl. G01N 33/00

(52) U.S. Cl. 436/94

(57) ABSTRACT

A system for profiling a genomic sequence comprising assigning modules to a genome, wherein each module has a defined sequence characteristic and the genome is divided into modules; assigning a value or weight to a module for a given profile, wherein the presence of one or more modules in a genomic sequence contributes to the profile of the genomic sequence relative to its value or weight; analysing a genomic sequence to identify modules present; and assigning a profile to the genomic sequence based on the presence of the modules and their respective value or weight.

### TECHNICAL FIELD

The invention relates to systems for profiling genomic sequences.

### BACKGROUND

The identification of genes responsible for human disease is useful to gain an understanding of disease mechanisms and is essential in the development of diagnostics and therapeutics. Linkage analysis of disease inheritance patterns is a successful procedure to associate a disease with a specific genomic region. Unfortunately, isolating the disease-causing gene(s) can be difficult: genomic regions are often large, containing hundreds of candidate genes, making experimental methods time consuming and expensive. Furthermore, searches for single nucleotide polymorphisms (SNPs) in the genomes of individual patients from clinical studies will produce a large number of potential gene candidates. These high-throughput analyses will require computational approaches to identify good candidates for further study.

The completion of the human genome sequencing project has permitted the development of new genome-scale bioinformatics approaches to understand disease. While some progress has been made in candidate gene prediction, these systems can, at best, only claim modest pruning of the genes in a disease interval and result in false negatives around 50% of the time.

Previous candidate gene prediction systems have largely been based on keyword similarity to known disease genes. For example, the G2D system is based on biomedical literature searches and associates pathological conditions with gene ontology (GO) terms. Candidate genes are then identified by homology to GO-annotated and disease-associated genes. The method POCUS finds candidate genes by identifying an enrichment of GO-keywords, shared InterPro domains and expression profiles among a given set of susceptibility loci relative to the genome at large. The method by Tiffin et al (Tiffin N, Kelso J F, Powell A R, Pan H, Bajic V B, Hide W A. (2005) Integration of text- and data-mining using ontologies successfully selects disease gene candidates. Nucleic Acids Res. 33, 1544-52) selects candidates according to their expression profiles within tissues associated with disease, and relationships between clinical and molecular data are identified using the eVOC anatomy ontology. The recent method SUSPECTS again compares GO, InterPro and expression libraries of putative disease genes with those known to be involved in the same disease. Similarly, GeneSeeker integrates keyword data based on mapping, expression and phenotypic databases from human and mouse studies. The method by Freudenberg and Propping (Freudenberg J, Propping P. (2002) A similarity-based method for genome-wide prediction of disease-relevant human genes. Bioinformatics., 18 S2, S110-5) is based on a measure of phenotypic similarity between diseases and produces clusters of disease genes using keywords derived from OMIM (Hamosh A, Scott A F, Amberger J, Bocchini C, Valle D, McKusick V A. (2002) Online Mendelian Inheritance in Man (OMIM), a knowledgebase of human genes and genomic disorders. Nucleic Acids Res., 30, 52-5). Recently, Franke et al 2006 (Franke L, Bakel H, Fokkens L, de Jong E D, Egmont-Petersen M, Wijmenga C. (2006) Reconstruction of a functional human gene network, with an application for prioritizing positional candidate genes. Am J Hum Genet. 78, 1011-25) developed a system based on predicted protein-protein interactions (PPIs), whereby disease genes are identified through common interactions to proteins in multiple disease intervals that have common phenotypes.

Some of these methods have been incorporated into a consensus approach that has been applied to select candidates for the complex diseases type 2 diabetes and obesity. Using a combination of methods appears to be effective for ranking candidate disease genes.

The present inventors have developed a computational system (termed ‘Common Module Profiling’ (CMP)) to predict profiles such as candidate disease genes within disease loci. These predicted disease genes, and their biochemical pathways, may constitute potential drug targets for the treatment of disease.

### SUMMARY OF INVENTION

In a first aspect, the present invention provides a system for profiling a genomic sequence comprising:

(a) assigning modules to a genome, wherein each module has a defined sequence characteristic and the genome is divided into modules;
(b) assigning a value or weight to a module for a given profile, wherein the presence of one or more modules in a genomic sequence contributes to the profile of the genomic sequence relative to its value or weight;
(c) analysing a genomic sequence to identify modules present; and
(d) assigning a profile to the genomic sequence based on the presence of the modules and their respective value or weight.

Preferably, the genomic sequence is an amino acid sequence of a protein and each module is a universal re-occurring unit found in protein sequences.

Preferably, the genome forms the encoding region and the encoding region is divided into different modules.

In a second aspect, the present invention provides a system for profiling an amino acid sequence to identify an associated profile, the system comprising:

(a) assigning modules to the protein coding region of a genome to divide the genome into modules, wherein each module has a defined amino acid characteristic;
(b) assigning a value or weight to a module for a given profile, wherein the presence of one or more modules in an amino acid sequence contributes to the profile of the sequence relatively to its value or weight;
(c) analysing an amino acid sequence to identify modules present; and
(d) assigning a profile to the amino acid sequence based on the presence of the modules and their respective value or weight.

The profile may be any useful information such as a gene or loci associated with a phenotype, disease, drug-binding characteristic, trait associated to pharmacogenomics, associated interacting genes, association with a phenotype, associated or interacting modules, or the module with a particular disease or phenotype, or associated biochemical pathways, or associated modules within biochemical pathways or interacting models with profiles with characteristics described herein.

In a preferred embodiment, the phenotype is a disease or a quantitative trait locus (QTL).

In another preferred embodiment, the profile is an association with a disease.

In another preferred embodiment, the profile is a drug-binding characteristic.

In one preferred embodiment, a given value or weight of a module assigned to a profile is obtained by identifying modules associated with a given phenotype (directly or indirectly through pathways or complexes) and assigning a score based on the similarity of a module to modules associated with a specific phenotype.

In another preferred embodiment, a given value or weight of a module assigned to a profile is obtained by identifying enrichment of those modules in loci (genomic regions) known to be associated with the phenotype. For example, this can be carried out by identification of overrepresentation of particular modules in loci associated with the phenotype and score the degree of overrepresentation.

The present inventors have carried out detailed analysis of genomic regions using proprietory software that can assign a value or weight to a module for a given profile. The present invention can thus identify modules in genomic sequences wherein each module has a defined sequence characteristic, associate profiles with the modules, and assign profiles to genomic sequences from the values or weights of the modules present.

For a given profile, typically a module is assigned a value or weight according to its presence in sequences associated with the profile.

In a third aspect, the present invention provides a system in computer readable form containing modules with defined genomic sequence characteristics wherein each module has an assigned value or weight for one or more profiles.

In a fourth aspect, the present invention provides a system in computer readable form containing modules with defined amino acid characteristics wherein each module has an assigned value or weight for one or more profiles.

In a fifth aspect, the present invention provides a system for profiling a genomic sequence comprising:

a data processing apparatus comprising a central processing unit (CPU),

a memory operably connected to the CPU, the memory containing a program adapted to be executed by the CPU,

wherein the CPU and memory are operably adapted to use inputted biological information to:

(a) assign modules to a genome, wherein each module has a defined sequence characteristic and the genome is divided into modules;
(b) assign a value or weight to a module for a given profile, wherein the presence of one or more modules in a genomic sequence contributes to the profile of the genomic sequence relative to its value or weight;
(c) analyse a genomic sequence to identify modules present; and
(d) assign a profile to the genomic sequence based on the presence of the modules and their respective value or weight.

In a sixth aspect, the present invention provides a system for profiling an amino acid sequence to identify an associated profile, the system comprising:

a data processing apparatus comprising a central processing unit (CPU),

a memory operably connected to the CPU, the memory containing a program adapted to be executed by the CPU,

wherein the CPU and memory are operably adapted to use inputted biological information to:

(a) assign modules to the protein coding region of a genome to divide the genome into modules, wherein each module has a defined amino acid characteristic;
(b) assign a value or weight to a module for a given profile, wherein the presence of one or more modules in an amino acid sequence contributes to the profile of the sequence relatively to its value or weight;
(c) analyse an amino acid sequence to identify modules present; and
(d) assign a profile to the amino acid sequence based on the presence of the modules and their respective value or weight.

In some preferred embodiments, the system of the fifth or of the sixth aspect of the invention further includes a web server operably connected to the data processing apparatus. In some such embodiments, the web server may facilitate the prediction or prioritization of candidate disease genes for both Mendelian and complex diseases.

In a seventh aspect, the present invention provides a computer program element comprising a computer program code to make a programmable device profile a genomic sequence by:

(a) assigning modules to a genome, wherein each module has a defined sequence characteristic and the genome is divided into modules;
(b) assigning a value or weight to a module for a given profile, wherein the presence of one or more modules in a genomic sequence contributes to the profile of the genomic sequence relative to its value or weight;
(c) analysing a genomic sequence to identify modules present; and
(d) assigning a profile to the genomic sequence based on the presence of the modules and their respective value or weight.

According to an eighth aspect, the present invention provides a computer program element comprising a computer program code to make a programmable device profile an amino acid sequence to identify an associated profile by:

(a) assigning modules to the protein coding region of a genome to divide the genome into modules, wherein each module has a defined amino acid characteristic;
(b) assigning a value or weight to a module for a given profile, wherein the presence of one or more modules in an amino acid sequence contributes to the profile of the sequence relatively to its value or weight;
(c) analysing an amino acid sequence to identify modules present; and
(d) assigning a profile to the amino acid sequence based on the presence of the modules and their respective value or weight.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of each claim of this specification.

In order that the present invention may be more clearly understood, preferred embodiments will be described with reference to the following drawings and examples.

### BRIEF DESCRIPTION OF THE DRAWINGS

FIG. 1 shows sensitivity (continuous line) and proportion of predicted genes that are actually disease genes (dashed line) for OPHID (diamond), OPHIDh (circle), OPHIDlit+ (triangle) and OPHIDlit− (square) at three levels of interactions (Distance). Results are shown for the 100 interval size only.

FIG. 2 shows performance of PPI data from a) OPHID, b) OPHIDh, c) OPHIDlit+ and d) OPHIDlit−. Results are shown for three levels of interaction using the shortest path length to a disease gene (Distance). Black diamonds represent the number of disease genes found. The number of non-disease genes returned at the 50-gene interval (square), 100-gene interval (triangle) and 150-gene interval (x). The number of disease genes returned by random selection at the 50-gene interval (*), 100-gene interval (circle) and 150-gene interval (+).

FIG. 3 shows CMP performance at different thresholds for the 100 gene interval size, based on ten diseases. Black bars represent the percentage of disease genes found. Gray bars represent the proportion of predictions that are actually disease genes.

FIG. 4 shows candidate gene enrichment for the 50 (a), 100 (b) and 150 (c) gene interval size. Black diamonds represent enrichment of data sets using the combined methods. Gray squares represent enrichment of data using random selection. Disease genes are listed alphabetically from left to right on the x-axis, as in Table 1.

FIG. 5 shows combined prediction success. a) Correct predictions based on known disease genes. b) Correct predictions based on multiple intervals c) Combined CPS and CMP predictions for familial hypertrophic cardiomyopathy (cfh). Disease genes are represented by their ENTREZ-name. Gene-linking lines are predictions by CPS and CMP. PRKAG2 and TPM1 where found using PPI data at a distance of three, all others found by PPI data were found at a distance of one.

FIG. 6 shows SNP-gene mapping approaches and genome coverage. (A) Nearest neighbour (NN) approach showing a resident SNP, the green shading representing the nearest gene, and the genes adjacent SNPs shaded in yellow. Bystander (BY) approach with colored shadings representing different interval sizes. SNPs are marked with blue bars. The number of SNPs captured by each approach is listed in Table 4. (B) Affymetrix 500K chip sets SNP to annotated gene coverage of the present invention. Total number of genes in the present invention is 27,499 (excluding genes on chromosomes X and Y). * common GWAS approach.

FIG. 7 shows a smoothed density distribution plot showing enrichment of genes similar to phenotype-specific known disease genes by CMP in the search space (colored lines) against the whole genome (black line) for (A) BD, (B) CAD, (C) CD, (D) HT, (E) RA, (F) T1D and (G) T2D. Search spaces shown are those of the MWS (dashed) and WS data sets (solid) for different SNP to gene mappings: nearest NN mapping (red), adjacent NN mapping (orange) and 1 Mbp BY mapping (blue).

FIG. 8 is a diagram illustrating overlap of remodelling genes (A) in five phenotypes CAD, HT, RA, T1D and T2D focusing on calpains and metalloproteases (ADAMs, ADAMTSs and MMPs); (B) in three phenotypes CAD, HT, and T2D.

### MODE(S) FOR CARRYING OUT THE INVENTION

A bioinformatics approach that encompasses methods of sequence comparison and protein pathway and interaction data analysis has been developed by the present inventors. Two methods may be used for the automated prediction of disease genes within known disease intervals.

Both methods use two sources of input for disease-gene prediction: firstly, known disease genes are used to predict novel disease genes in intervals of the same disease-phenotype and secondly, without knowledge of the disease-genes, all the genes in the multiple intervals of the same phenotype are used to find protein relationships to predict candidate disease genes.

The first method and useful part of the present invention, Common Module Profiling (CMP), is based on the principle that candidate genes may have similar functions to disease genes that have already been determined. This is analogous in concept to methods using functional annotations, but many human proteins lack annotation and, therefore, similarities would be missed when comparing keywords alone. For example, only 10,000 human proteins, approximately 25% of the human proteome, have manually curated GO-terms.

CMP uses a domain-based (modules) comparative sequence analysis to identify those proteins with potential functional-similarity. Domain based sequence comparison searches have been shown to be more accurate than full-sequence searches as commonly applied in BLAST or PSI-BLAST database searches. Unlike the keyword systems, CMP calculates a measure of domain-based similarity to known disease genes rather than a binary comparison.

For the CMP algorithm, complete protein domain annotation is performed by parsing all protein sequences against the Pfam library of Hidden Markov models using HMMer. Pairwise similarity scores between common domains of proteins are calculated using the Smith-Waterman algorithm implemented in SSEARCH. The alignments are scored using a metric based on the normalized bit score, which ranges between 0 and 1. Candidate genes above a given threshold—selectable by the user—are prioritized based on this score. Domain combinations are tested for over-representation in the intervals compared to the genome as a whole through upper and lower significance tests, based on a range of expected values relating to domain correlation. The upper significance test is based on the assumption of no correlation between domains, while the lower significance test is based on the assumption of complete correlation. For all domain combinations the real degree of domain correlation will lie between these two scenarios. A χ² value is calculated for each scenario, and the resulting candidate genes are ranked based on these values.

In known gene mode, candidate proteins are compared with known phenotype-associated proteins. In ab initio mode, a census of all domains in input intervals associated with the phenotype is taken, and over-representation of specific domain combinations amongst genes from different intervals is tested.

The second method, Common Pathway Scanning (CPS), is based on the assumption that common phenotypes are generally associated with disruption in proteins that participate in the same complex or pathway. Recently, Gandhi et al 2006 (Gandhi T K, Zhong J, Mathivanan S, Karthick L, Chandrika K N, Mohan S S, Sharma S, Pinkert S, Nagaraju S, Periaswamy B (2006) Analysis of the human protein interactome and comparison with yeast, worm and fly interaction datasets. Nature Genet. 38, 285-93) showed that disease-genes preferentially interact with other disease-causing genes. There are currently over 200 biological pathway and network resources available. The present inventors have utilised data from BioCarta (www.biocarta.com), KEGG and OPHID, the most comprehensive databases of their type. BioCarta and KEGG are chiefly pathway databases with BioCarta specialising in signalling pathways and KEGG in metabolic pathways. OPHID is a secondary PPI database containing literature-derived interaction data from BIND, MINT and HPRD, as well as data from recent high-throughput experimentation. OPHID also contains transferred interactions from orthologous proteins in model organisms.

The CPS algorithm uses the phenotype-specific disease genes to associate pathways with the phenotype. In known disease gene mode, the genes within candidate loci are checked for their occurrence in disease phenotype-associated pathways. For each disease, pathways are ranked by the number of known disease genes that they contain and candidate genes are ranked according to the disease-relevance of their associated pathways.

Under multiple interval or ab initio mode, the pathways of all genes in the intervals are pooled and tallied in order to identify the most common A pathway is only counted once for each locus, even if multiple pathway-associated genes are found within the locus. Candidate disease genes are then identified according to the pathway frequency across loci.

Linkage analysis is a successful procedure to associate disease with specific genomic regions. Unfortunately, these regions are often large, containing hundreds of genes, which make experimental methods employed to identify the disease gene arduous and expensive. It is important, therefore, to prioritise likely disease genes and discount those that are unlikely to be involved in the disease. We present a computational approach to prioritise candidate disease genes for further experimental study. Starting with a disease interval, two algorithms can be applied: Common Module Profiling (CMP) and Common Pathway Scanning (CPS), which are computational versions of traditional approaches to candidate selection. CPS applies network data derived from protein-protein interaction and pathway databases to identify relationships to known disease genes. CPS is based on the assumption that common phenotypes are associated with dysfunction in proteins that participate in the same complex or pathway. CMP identifies likely candidates using a domain-dependent sequence similarity approach, based on the hypothesis that disruption of genes of similar function will lead to the same phenotype. Both methods, CMP and CPS may also be combined for the automated prediction of disease genes within known disease intervals. Both algorithms use two forms of input data: known disease genes or multiple disease loci. When using known disease genes as input, our combined methods have a sensitivity of 0.518 and a specificity of 0.966 and reduced the candidate list by 13-fold. Using multiple loci, our methods successfully identify disease genes for all benchmark diseases with a sensitivity of 0.835 and a specificity of 0.626. Our combined approach also prioritizes good candidates and will accelerate the disease gene discovery process.

### Materials and Methods

### Annotation Pipeline

All biological data was combined into a relational database. For examples 1 and 2, human disease gene information was extracted from the OMIM database and lists of genes flanking the disease genes were obtained from EntrezGene (build 35). Protein sequence data was taken from GenBank and complete protein domain annotation was performed on all protein sequences using Pfam Hidden Markov Models (version 18). Finally, all genes were mapped to the latest pathway and PPI data downloaded from BioCarta, KEGG and OPHID.

### Common Module Profiling

CMP compares the Pfam-domain content of each protein within a disease interval to identify putative disease genes. Different calculations are performed depending on whether CMP uses known disease genes or multiple intervals as input.

When known disease genes are used as input, a protein (candidate) observed to have disease-like domains is assigned a score (S) based on the similarity between the protein's domains (j) and the domains (i) in the known disease gene (dg) using SSEARCH bit scores(s). SSEARCH is an implementation of the Smith and Waterman local alignment algorithm. Scores were normalised by matching the equivalent region of the disease gene against itself on a domain by domain basis (equation 1).

$$S = \frac{{\sum }_{i} max ( s ( {dg}_{i} , {candidate}_{j} ) )}{{\sum }_{i} s ( {dg}_{i} , {dg}_{i} )} j = 1 … N \qquad ( 1 )$$

Where a protein has multiple domains of the same type, the highest scoring matching domain is used.

When CMP is used across multiple intervals, a census of all domains in every interval associated with the disease is taken. A similarity score based on the numerator of equation 1 is calculated as well as two calculations of statistical significance. In the first calculation of significance, domains in a sequence are assumed to be completely uncorrelated, this represents an upper limit of significance. The expected (eₐ) number of genes containing those domains is calculated by:

$${e}_{a} = mnf {\prod }_{i} {P}_{i} \qquad ( 2 )$$

where m is the number of intervals containing the domains of interest; n is the number of genes in the interval; and f is a form factor, related to the average number of domains per gene. The probability of encountering domain i is given by:

$${P}_{i} = \frac{{N}_{i}}{N} \qquad ( 3 )$$

where N is all domain types. These numbers are determined from a census of all domains across the genome. For the second calculation of significance, domains are assumed to be completely correlated, this represents a lower limit of significance. The expectation (e_{b}) is based on the prevalence of the rarest domain:

e_{b}=mnf.min(Pi) (4)

Two χ² tests (χ²c and χ²b) are then calculated in the usual manner using the two expectation values at a significance of 0.995. Clusters of genes containing the same domains are then ranked according to the two alternative values.

### Common Pathway Scanning

Potential disease genes were predicted by identifying all proteins within a disease interval that are part of a pathway, described in BioCarta and KEGG. PPI data from OPHID was used to identify novel disease genes by identifying the interaction partners of known disease genes in a disease interval. Three levels of interactions are tested for potential disease genes, based on the shortest path length to a disease gene. When CPS is applied across multiple intervals, i.e. in the absence of known disease genes, all interaction partners and pathways associated with the genes in each interval are compared. Disease genes are predicted by identifying common pathways or interaction partners between the intervals.

### Benchmarking

The prediction algorithms were validated using data from previously determined disease intervals where at least three disease genes have been identified. The disease genes are used to generate pseudo-intervals. Three pseudo-interval sizes are used that encompass 50, 100 and 150 genes around the known disease genes.

When the disease genes were used as the input, the predictive power of each algorithm was tested on each disease gene using leave-one-out cross validation. In this method, one of the disease genes was disregarded and the remaining known disease genes were used to identify the omitted disease gene in its pseudo-interval. If there is not information about the disease genes, all genes in the intervals sharing a phenotype were used to identify common relationships.

Several measures of predictive power were used: sensitivity, the probability of finding a disease gene among disease genes (TP/(TP+FN)); and specificity, the probability of not finding a disease gene among non-disease genes (TN/(TN+FP)); where TP is the number of true positives, TN is the number of true negatives, FP is the number of false positives and FN is the number of false negatives. An enrichment ratio (ER) was also calculated for each disease from the proportion of disease genes predicted by the methods divided by the proportion of disease genes within the disease intervals (equation 5).

$$ER = \frac{TP / ( TP + FP )}{( \sum disease genes / \sum all genes )} \qquad ( 5 )$$

CPS and CMP predictions were compared with a random selection of candidate genes within a disease interval. The number of random assignments made was based on the number of predictions made by each method. Random selections were performed 1000 times for each disease, from which an average number of correctly identified disease genes is calculated.

### Results

### Example 1

### Candidate Gene Prediction Using Each of the Two Methods (CPS and CMP)

Table 1 shows the results of candidate gene prediction for each of the two methods on the 29 diseases as used by Turner et al. (Turner F S, Clutterbuck D R, Semple C A. (2003) POCUS: mining genomic sequence annotation to predict disease genes. Genome Biol., 4, R75) in their analysis of POCUS. Complete lists of all disease genes and pseudo-intervals used for benchmarking are available at our web site www.pathologene.org. The present invention made predictions for all 29 diseases in each of the 50, 100 and 150-gene intervals and correctly predict a disease gene in 20 of the 29 diseases, finding 88 of the total 170 disease genes. In comparison, POCUS made candidate predictions for eight of the 29 diseases for interval sizes averaging 94 genes and only five of the diseases had a disease gene correctly predicted.

CMP results are based on a cut-off threshold of 0.1. CPS-interactions go to the 1st level of interaction only. CPS-OHPID contains all PPI data from OPHID. CPS-OPHIDh contains human data only. CPS-OPHIDlit+ contains data from literature databases only. CPS-OPHIDlit− does not contain PPI data from literature databases. Random is calculated on total predictions for the 50, 100 and 150 interval sizes. Disease abbreviations: aan, adrenoleukodystrophy, autosomal neonatal; alz, Alzheimer disease; aml, acute myeloid leukemia; bb, Bardet-Biedl syndrome; bc, breast cancer; bcc, basal cell carcinoma; cchn, colorectal cancer, hereditary nonpolyposis; cf, cystic fibrosis; cfh, cardiomyopathy, familial hypertrophic; cmt, Charcot-Marie-Tooth disease; ebl, epidermolysis bullosa letalis; ed, epiphyseal dysplasia, multiple types 1-5; fap, familial adenomatous polyposis; gc, gastric cancer; h, hypertension; ibd, inflammatory bowel disease; joag, juvenile-onset primary open angle glaucoma; lca, Leber congenital amaurosis; lhscr, long-segment Hirschsprung disease; md, muscular dystrophy, limb-girdle; mf, familial meningioma; mody, maturity-onset diabetes of the young; niddm, type 2 diabetes mellitus; oc, ovarian carcinom; pc, prostate cancer; pd, Parkinson disease; rp, retinitis pigmentosa; sle, systemic lupus erythematosus; tcp, thyroid carcinoma, papillary.

TABLE 1
Number of correctly predicted disease genes by each method using known disease genes.

| | Known | Successful Automated Predictions | | | | | | | | | | |
|---|---|---|---|---|---|---|---|---|---|---|---|---|
| | Disease | | CPS | | | | | | | Random | | |
| Disease | Genes | CMP | BioCarta | KEGG | OPHID | OPHIDh | OPHIDlit+ | OPHIDlit− | Total | 50 | 100 | 150 |
| aan | 4 | 0 | 0 | 0 | 3 | 3 | 3 | 2 | 3 | 0.1 | 0.1 | 0.1 |
| alz | 8 | 2 | 3 | 6 | 5 | 5 | 5 | 3 | 6 | 0.3 | 0.2 | 0.2 |
| aml | 4 | 0 | 0 | 0 | 0 | 0 | 0 | 0 | 0 | 0.2 | 0.2 | 0.2 |
| bb | 4 | 0 | 0 | 0 | 0 | 0 | 0 | 0 | 0 | 0.0 | 0.0 | 0.0 |
| bc | 9 | 0 | 4 | 0 | 6 | 6 | 6 | 0 | 6 | 0.5 | 0.5 | 0.5 |
| bcc | 4 | 1 | 1 | 2 | 3 | 3 | 3 | 0 | 3 | 0.1 | 0.0 | 0.1 |
| cchn | 6 | 5 | 0 | 0 | 5 | 4 | 4 | 4 | 5 | 0.4 | 0.3 | 0.3 |
| cf | 5 | 0 | 2 | 2 | 0 | 0 | 0 | 0 | 2 | 0.2 | 0.2 | 0.2 |
| cfh | 12 | 5 | 0 | 4 | 4 | 4 | 4 | 0 | 9 | 1.0 | 0.7 | 0.8 |
| cmt | 5 | 0 | 0 | 0 | 2 | 2 | 2 | 0 | 2 | 0.2 | 0.2 | 0.2 |
| ebl | 5 | 3 | 0 | 5 | 5 | 5 | 5 | 0 | 5 | 0.2 | 0.1 | 0.1 |
| ed | 7 | 5 | 0 | 2 | 0 | 0 | 0 | 0 | 5 | 0.4 | 0.3 | 0.2 |
| fap | 4 | 0 | 0 | 3 | 0 | 0 | 0 | 0 | 3 | 0.2 | 0.2 | 0.1 |
| gc | 5 | 0 | 2 | 3 | 0 | 0 | 0 | 0 | 4 | 0.3 | 0.2 | 0.2 |
| h | 5 | 0 | 0 | 0 | 0 | 0 | 0 | 0 | 0 | 0.1 | 0.2 | 0.2 |
| ibd | 5 | 0 | 2 | 3 | 4 | 4 | 4 | 2 | 4 | 0.4 | 0.3 | 0.3 |
| joag | 4 | 0 | 0 | 0 | 0 | 0 | 0 | 0 | 0 | 0.1 | 0.1 | 0.1 |
| lca | 6 | 0 | 0 | 0 | 0 | 0 | 0 | 0 | 0 | 0.1 | 0.1 | 0.1 |
| lhscr | 5 | 0 | 0 | 2 | 2 | 2 | 2 | 0 | 4 | 0.2 | 0.3 | 0.3 |
| md | 6 | 2 | 0 | 0 | 2 | 2 | 2 | 0 | 3 | 0.1 | 0.1 | 0.1 |
| mf | 4 | 0 | 0 | 0 | 0 | 0 | 0 | 0 | 0 | 0.2 | 0.2 | 0.2 |
| mody | 6 | 2 | 0 | 0 | 4 | 4 | 4 | 2 | 5 | 0.3 | 0.3 | 0.3 |
| niddm | 8 | 4 | 2 | 0 | 2 | 2 | 2 | 2 | 5 | 0.6 | 0.4 | 0.3 |
| oc | 4 | 0 | 0 | 4 | 2 | 2 | 2 | 2 | 4 | 0.3 | 0.3 | 0.3 |
| pc | 6 | 0 | 0 | 0 | 0 | 0 | 0 | 0 | 0 | 0.1 | 0.1 | 0.2 |
| pd | 3 | 0 | 0 | 3 | 2 | 2 | 2 | 0 | 3 | 0.1 | 0.0 | 0.0 |
| rp | 10 | 0 | 0 | 0 | 0 | 0 | 0 | 0 | 0 | 0.2 | 0.2 | 0.2 |
| sle | 3 | 0 | 0 | 0 | 0 | 0 | 0 | 0 | 0 | 0.2 | 0.1 | 0.2 |
| tcp | 13 | 3 | 0 | 2 | 4 | 4 | 4 | 0 | 7 | 0.9 | 0.8 | 0.8 |
| Total | 170 | 32 | 16 | 41 | 55 | 54 | 54 | 17 | 88 | 8.0 | 6.6 | 6.7 |

CMP Benchmark Performance from Known Disease Genes

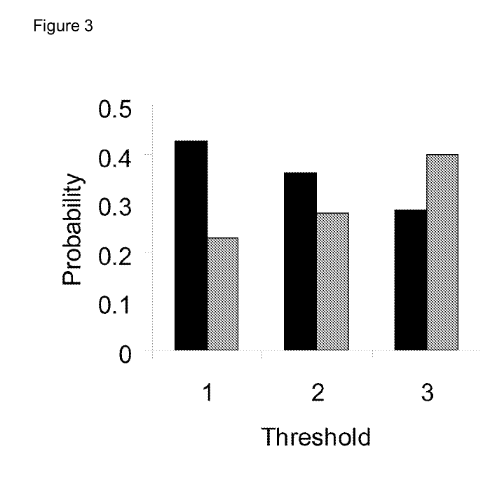

CMP identifies disease genes using domain-based comparative sequence analysis. This was achieved by first using Pfam Hidden Markov Models to annotate the domain content of known disease genes. Putative disease genes were then identified based on a shared domain content with the known disease genes. FIG. 3 shows the performance of CMP at three score thresholds for the 100-gene gene interval. The ratio of true positives to false positives was best at a threshold of 0.4. However, at a threshold of 0.1, CMP found more disease genes and sensitivity was at its best. At this threshold, 7.5%, 11.6% and 18.5% of predictions are disease-causing genes for the 50, 100 and 150-gene intervals, respectively. Less than 0.8% of proteins rejected will be disease genes.

Independently, CMP correctly predicts 32 disease genes for 10 diseases at a score threshold of 0.1 and has a sensitivity of 0.2 and a specificity of 0.98 for each interval size. Overall enrichment for all diseases was 11-fold at the 100-gene interval size.

### CMP Benchmark Performance Using Multiple Intervals

When multiple loci were used as the input to CMP, a census of the domain content of all genes in the specified loci was taken. The numbers of genes with a specific domain content were compared with the expected number of genes based on the prevalence of those domains in the genome (see Materials and Methods detailed above). Clusters of genes with similar domain content were ranked based on two estimates of the significance: the first assumed that the domain content of the cluster is completely uncorrelated and is an upper estimate of the significance (χ²a); the second assumed the domains are highly correlated and the prevalence is determined by the rarest domain (χ²b). These two values are the same for single domain proteins.

Comparison of the CMP results are shown in Table 2. Results have been split into subgroups: those that contain multiple Pfam domains (multi) and those that contain at least one Pfam domain (all). Sensitivity is low for the multidomain method because disease genes with zero or one Pfam domain are included in the false negatives. However, the specificity was very high indicating that if the target disease genes were multiple domain proteins, the method is very effective.

The 36 disease genes potentially identifiable by CMP, based on their domain similarity, can be divided into 16 clusters, containing two or more disease genes. Of these genes, 32 were identified by CMP using known disease genes as a starting point, while four fell below the 0.1 threshold similarity. Using multiple intervals as input, two clusters containing four genes were not found as determined by significance. For example, genes RET and NTRK1 involved in thyroid carcinoma have a protein kinase domain in common, but protein kinase domains are very common in the genome and thus lowered the significance of the shared domain.

Of the 14 successfully identified gene clusters, 11 were ranked in the top 10 for that disease based on either score of significance and 13 were in the top 20. The χ²a test favours multi-domain proteins whereas disease genes that are single domain proteins have a better chance of being detected with χ²b.

### CPS Benchmark Performance Using Known Disease Genes

CPS identifies novel disease genes by finding proteins that are linked with the product of a known disease gene in the pathway and PPI databases. Results for CPS are divided into three datasets: pathway data from BioCarta, pathway data from KEGG and PPI data from OPHID. KEGG pathway data correctly predicts 41 disease genes in 13 diseases. For the 100-gene interval size, the probability of finding a disease gene (sensitivity) using KEGG data is 0.257, and the probability of not finding a disease gene among non-disease genes (specificity) by KEGG is 0.981. Overall data enrichment is 12-fold for the 100-gene interval size.

BioCarta pathway data identifies 16 disease genes in seven diseases. BioCarta has a sensitivity of 0.152, a specificity of 0.992 and an enrichment of 16-fold for the 100-gene interval size. The complementary nature of these pathway databases is demonstrated by their unique results. BioCarta finds disease genes for two diseases, type 2 diabetes mellitus and breast cancer, where KEGG fails. KEGG finds disease genes for eight diseases where BioCarta fails.

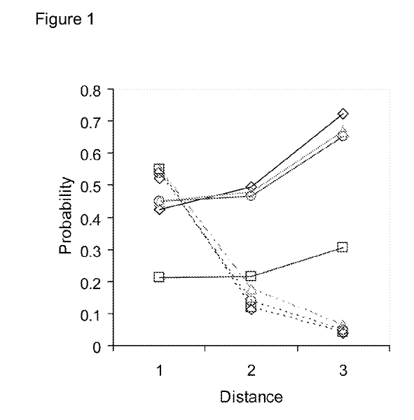

The OPHID PPI dataset contains 48,321 interactions for 10,666 proteins representing 13% of the estimated complete human-interactome. Overall, OPHID has a sensitivity of 0.423, a specificity of 0.996 and an enrichment of 50-fold at the 100-gene interval size. These results are much better than the pathway data, but the success of prediction using PPI data might be influenced by PPI data derived from literature associations of well studied diseases. In an attempt to remove bias from literature PPIs and to assess the usefulness of orthology data, OPHID is further split into several overlapping sets: human-only data, i.e. the data does not contain transferred orthologous interactions (OPHIDh); PPI data derived from literature searches only, i.e. data from the BIND, HPRD and MINT databases (OPHIDlit+); and all PPIs except those from the literature databases (OPHIDlit−). The difference between OPHID and OPHIDh predictions is small: OPHID finds one more disease gene than OPHIDh, but with slightly more false positives. FIG. 1 shows the sensitivities for each of the datasets compared with the proportion of correct predictions at increasing path lengths for the 100-gene interval size. At the first level of interactions the majority of correct predictions, 54, is found using the OPHIDlit+set, with a sensitivity of 0.45 and specificity of 0.996. The non-literature PPIs find 17 disease genes, with a sensitivity of 0.213 and a specificity of 0.996. While the probability of finding a disease gene is lower in the non-literature set, overall data-enrichment is the same, 53-fold, and the proportion of correct predictions is the same, 0.55. Therefore, it is the larger coverage of the literature data that gives it the advantage over the non-literature set and suggests that the experimental data and orthology data held in the OPHIDlit− set is of equal quality to the literature assignments.

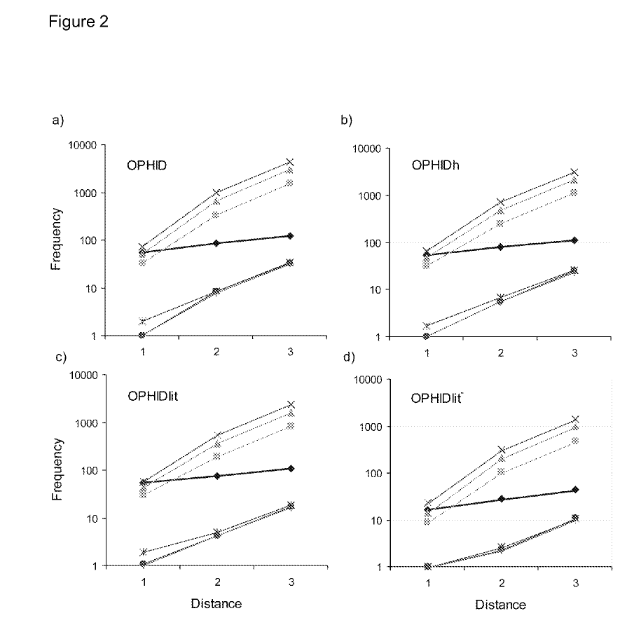

FIG. 2 shows the number of false positives returned by the interaction data at increasing path lengths up to a distance of three interactions from the known disease genes. As the shortest path length increases the sensitivity improves but the number of false positives increases exponentially reducing specificity. At a distance of two interactions, the full OPHID set finds 84 disease genes with a sensitivity of 0.494, a specificity of 0.96 and an enrichment of 11-fold. Increasing the distance to three interactions, finds 123 disease genes, with a high sensitivity of 0.723, but a smaller specificity of 0.816 and a poor four-fold enrichment.

Combining the results from the full OPHID set (where the shortest path length is one) with the results from BioCarta and KEGG, CPS makes predictions for 28 diseases and identifies 78 disease genes. Overall CPS performance has a sensitivity of 0.47 with a specificity of 0.977 and an enrichment of 17-fold at the 100-gene interval size. Less than 0.6% of proteins rejected will be disease genes.

### CPS Benchmark Performance Using Multiple Intervals

When multiple loci are used as the input to CPS, 100 disease genes were correctly identified in the 100-gene intervals. While sensitivity was high 0.588, more false positives were predicted compared to input from known disease genes. This reduced specificity to 0.844 and the enrichment ratio to 3.7-fold. The pathway and PPI data complement each other: CPS using pathway data alone finds 28 disease genes that are missed by the PPI data. Conversely, CPS using PPI data alone finds 33 disease genes that the pathway data misses and together they find the same 39 disease genes. In the absence of known disease genes, the use of network data on multiple disease-loci is a powerful approach to identify disease genes. Table 2 shows the results for each of the individual methods.

TABLE 2
Multiple loci benchmark results.

| | 50 | | | 100 | | | 150 | | |
|---|---|---|---|---|---|---|---|---|---|
| Method | Sens. | Spec. | ER | Sens. | Spec. | ER | Sens. | Spec. | ER |
| CPS-Pathway | 0.353 | 0.903 | 3.4 | 0.394 | 0.886 | 3.4 | 0.406 | 0.875 | 3.2 |
| CPS-PPI | 0.394 | 0.953 | 7.3 | 0.424 | 0.934 | 6.1 | 0.471 | 0.919 | 5.6 |
| CPS | 0.541 | 0.873 | 4.0 | 0.588 | 0.844 | 3.7 | 0.624 | 0.824 | 3.5 |
| CMP (X²a | 0.165 | 0.953 | 3.3 | 0.188 | 0.941 | 3.1 | 0.229 | 0.929 | 3.2 |
| multi) | | | | | | | | | |
| CMP (X²a all) | 0.459 | 0.769 | 1.9 | 0.553 | 0.715 | 1.9 | 0.588 | 0.688 | 1.9 |
| CMP (X²b | 0.159 | 0.954 | 3.2 | 0.176 | 0.944 | 3.1 | 0.218 | 0.935 | 3.3 |
| multi) | | | | | | | | | |
| CMP (X²b all) | 0.459 | 0.770 | 2.0 | 0.553 | 0.716 | 1.9 | 0.582 | 0.690 | 1.9 |
| CPS-CMP | 0.741 | 0.692 | 2.3 | 0.835 | 0.626 | 2.2 | 0.865 | 0.592 | 2.1 |
| (X²a all) | | | | | | | | | |

### Combined CMP and CPS Methods

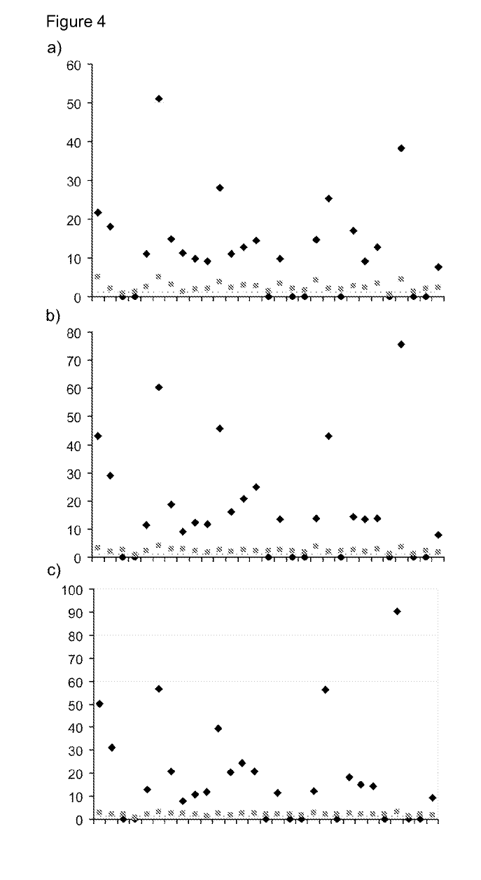

FIG. 4 shows the enrichment scores for each disease using the combined methodology. The combined methods are better than random selection in 20 of the diseases and only worse than random when no correct predictions are made.

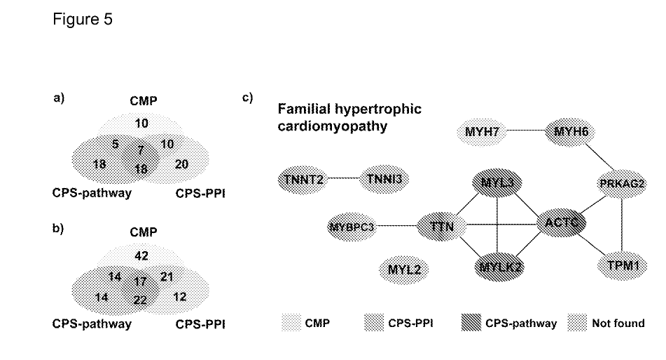

While each method was successful at identifying disease causing genes, performance was improved when combining the methods. The methods tend to be complementary, finding disease genes where the other methods fail: CPS identified disease genes for 10 diseases for which CMP found none and CMP identified nine disease genes that are missed by CPS (FIG. 5).

The probability of finding a disease gene can be increased when combining the results from the two methods: sensitivity increases to 0.512 with a specificity of 0.966 for the 50, 100 and 150-gene intervals. Of the rejected genes, only 0.5% will be disease genes. Overall enrichment is 11-fold in the 50-gene interval and 13-fold in the 100 and 150-gene intervals. Removing the literature-derived PPI data only slightly reduces overall performance: sensitivity is 0.424, selectivity is 0.967 and enrichment is 11-fold at the 100-gene interval. When extending the OPHID interaction data to the second level of interaction, overall sensitivity increases to 0.588, but with a reduction in both specificity, 0.934, and enrichment, eight-fold, for each interval size.

An example of the success of the combined methods can be seen for familial hypertrophic cardiomyopathy (cfh) (FIG. 5c). For the 12 known disease-genes, nine were found by CPS and CMP and a further two were found by the PPI data at a distance of three. Both CPS-PPI data and CMP identify disease genes through relationships between Titin (TTN) and myosin binding protein C (MYBPC3), and between Troponin I type 3 (TNNI3) and troponin T2 (TNNT2). CMP exclusively linked disease genes myosin heavy polypeptide 6 (MYH6) and myosin heavy polypeptide 7 (MYH7). The CPS-pathway-data from KEGG links actin (ACTC), myosin light polypeptide kinase 2 (MYLK2), myosin light polypeptide 3 (MYL3) and titin through the ‘regulation of actin cytoskeleton’ pathway.

For the combined multiple-interval predictions at the 100-gene interval, sensitivity greatly improves to 0.835, however specificity and enrichment to fall to 0.626 and 2.2-fold respectively.

### Example 2

### The Use of CMP and CAP to Select and Prioritize Valid Disease Candidates from the SNPs of Genome-Wide Association Studies (GWAS)

The Wellcome Trust Case-Control Consortium (WTCCC) data was an available valuable resource for the use of CMP and CAP to understand complex diseases. The WTCCC GWAS data contains a series of analyses on case-control studies who were known to have Bipolar Disorder (BD), or Coronary Artery Disease (CAD), or Crohn's Disease (CD), or Hypertension (HT), or Rheumatoid Arthritis (RA), or Type I Diabetes (T1D) or Type II Diabetes (T2D). The WTCCC GWAS used Affymetrix chip sets with approximately 500,000 known SNPs (Affy500k), with positions referenced to the human genome sequence assembly from NCBI (build 35). These SNPs map to 489,763 autosomal SNPs on the current genome assembly (build 36.3), and 459,231 SNPs following WTCCC quality control. The WTCCC data compromised 1,868 BD cases, 1,926 CAD cases, 1,748 CD cases, 1,952 HT cases, 1,860 RA cases, 1,963 T1D cases, 1,924 T2D cases, and 2,938 common controls.

A double sift approach was taken to assess the etiology of the WTCCC data by taking the best phenotype-associated SNPs and resifting the data using the biological knowledge base. The biological knowledge base employed utilized pathways and domain-based similarity to find relations between multiple genes associated with genetic data for specific phenotypes. As some previous studies have suggested the location of elements controlling genes may be distal to the actual transcripts and protein-coding regions themselves eg those on bystander genes, SNPs were mapped to genes in six different ways to investigate how these mappings affected predictions. Multiple predictions were made using the CMP and CPS methods of the present invention.

### SNP Filtering

An initial set of associated SNPs were filtered from the summary data of SNPTEST. SNPTEST is a program that performs a series of association tests on the genotypes obtained from the case-control studies. The p-value of the trend test statistic (Cochran-Armitage test) of the additive genetic model was used as an indicator of SNP significance. Four different p19 value thresholds were used to create four associated SNP data sets for each phenotype: a highly significant SNP set (HS, p<5×10⁻⁷), a moderately high significant set (MHS, p≦10⁻⁵), a moderately-weak significant set (MWS, p≦10⁻⁴), and a weakly significant set (WS, p≦10⁻³).

SNPs within the sets were clustered based on the physical distance to one another through a naïve clustering process. The naïve clustering process formed a cluster when a SNP was within about 50 Kbp of another SNP.

Associating SNPs with Positional Candidate Genes

SNPs were associated with genes using two major assumptions. The first assumption is that a disease-associated SNP is either resident in, or adjacent to, a disease gene and is termed the Nearest Neighbour (NN) approach. The second assumption is taken from previous studies investigating work on bystander genes and these previous studies suggest that a significant SNP may be near a disease gene but may not be the closest gene. For instance the fibroblast growth factor 8, FGF8, is controlled by regulatory elements within and beyond the neighboring FBXW4. In order to enable the present inventors to discover potential bystander genes an additional approach was utilised whereby genes were captured from intervals created around each SNP, and was termed the Bystander (BY) approach.

For the NN approach, three sets of genes were created: a set containing genes with SNPs internal to a gene boundary defined by the resident set (RefSeq); a second set with SNPs resident in a gene or a directly adjacent to it, termed the nearest set; and a third set with a SNPs was either resident in or directly adjacent to the four nearest genes, termed the adjacent set. The nearest set corresponds to a set commonly selected by NN approaches in most recent GWAS. In the adjacent set, genes on both strands of a chromosome were considered in both the 5′ and 3′ direction. For both the nearest and adjacent sets physical distance between a SNP and a gene was not used as a constraint.

For the BY approach, three different sized intervals were investigated by the present inventors. Genes on both strands around a SNPs were pooled from flanking intervals of about 0.1 Mbp, about 0.5 Mbp or about 1 Mbp in width.

### Prediction and Prioritization of Candidate Genes

To determine which SNPs were more likely to contribute to a disease phenotype, a set of analyses were performed using direct SQL queries of a web server housing an in-house database for analysis by CMP or CPS. Two modes of input were used the first was “known disease mode” and the second was “ab initio mode”. Both modes of input were used to determine the common properties of genes within the six gene sets (detailed above) for each disease. Known disease gene input mode was assisted by phenotype-associated genes from OMIM as seeds (Table 3). Ab initio input mode only used genes pooled from the intervals (about 0.1 Mbp, about 0.5 Mbp or about 1 Mbp in width). It is important to note that known disease data was defined prior to GWAS on the diseases, and therefore was restricted to OMIM entries.

TABLE 3
OMIM phenotype associated genes used as seeds for the known disease gene approach.

| Disease | Genes (HUGO) | Gene Entrez IDs | OMIM IDs |
|---|---|---|---|
| Bipolar Disorder (BD) | SLC6A3, XBP1, FKBP5, and | 6531, 7494, 2289, | 125480, 612371, |
| | HTR2A | 3356 | 608516 |
| Coronary Artery | ABCA1, MEF2A, LRP6, | 19, 4205, 4040, | 143890, 147545, |
| Disease (CAD) | CCL2, CX3CR1, LPA, IRS1, | 6347, 1524, 4018, | 152200, 158105, |
| | KL, PON1, PON2, MMP3, | 3667, 9365, 5444, | 168820, 185250, |
| | CD36, and NOS3 | 5445, 4314, 948, | 601470, 602447, |
| | | 4846 | 603507, 604824, |
| | | | 608320, 610938 |
| Crohn's Disease (CD) | IL23R, DEFB4, DLG5, | 149233, 1673, 9231, | 612261, 266600 |
| | CARD15, and IL6 | 64127, 3569 | |
| Hypertension (HT) | HSD11B2, NR3C2, PNMT, | 3291, 4306, 5409, | 145500, 108962, |
| | AGTR1, PTGIS, NPR3, | 185, 5740, 4883, | 124080, 125853, |
| | BMPR2, ACSM3, KCNMB1, | 659, 6296, 3779, | 145505, 178600, |
| | ADD1, AGT, ECE1, GNB3, | 118, 183, 1889, | 189800, 218030, |
| | RETN, NOS3, NOS2A, | 2784, 56729, 4846, | 265380, 605115, |
| | CYP3A5, CYP11B2, CPS1, | 4843, 1577, 1585, | 608622 |
| | SELE, ATP1B1, RGS5, and | 1373, 6401, 481, | |
| | EPHX1 | 8490, 2052 | |
| Rheumatoid Arthritis | STAT4, IL10, CD244, HLA- | 6775, 3586, 51744, | 180300, 604302 |
| (RA) | DRB1, CIITA, NFKBIL1, | 3123, 4261, 4795, | |
| | PADI4, PTPN22, RUNX1, | 23569, 26191, 861, | |
| | SLC22A4, MIF, and IL6 | 6583, 4282, 3569 | |
| Type I Diabetes (T1D) | IL6, TCF1, OAS1, FOXP3, | 3569, 6927, 4938, | 222100, 612522, |
| | ITPR3, PTPN22, IL2RA, | 50943, 3710, 26191, | 600320, 601388, |
| | CTLA4, CCR5 and SUMO4 | 3559, 1493, 1234, | 601942 |
| | | 387082 | |
| Type II Diabetes | PTF1A, TCF7L2, KCNJ11, | 256297, 6934, 3767, | 125853, 125851, |
| (T2D) | ABCC8, MAPK8IP1, UCP3, | 6833, 9479, 7352, | 601283, 609069, |
| | TCF1, IPF1, IRS2, LIPC, | 6927, 3651, 8660, | 601665 |
| | SLC2A4, TCF2, RETN, | 3990, 6517, 6928, | |
| | AKT2, GPD2, NEUROD1, | 56729, 208, 2820, | |
| | IRS1, CAPN10, PTPN1, | 4760, 3667, 11132, | |
| | PPARG, SLC2A2, IGF2BP2, | 5770, 5468, 6514, | |
| | WFS1, CDKAL1, ENPP1, | 10644, 7466, 54901, | |
| | IL6, GCK, PAX4, SLC30A8, | 5167, 3569, 2645, | |
| | and HNF4A | 5078, 169026, 3172 | |

Genes in each data set were prioritized based on common pathways (using the CPS method) and common domains (using the CMP method). For CPS, the pathways of known disease genes were compiled, and pathways containing at least two genes from distinct loci were ranked based on the total number of loci involved (see Materials and Methods detailed above). The number of genes in the pathway varied which may influence the likelihood of pathway commonality among the gene sets. To determine the likelihood of a pathway being associated with a phenotype, Fisher's exact test was calculated using R. Fisher's exact test is a statistical significance test used in the analysis of contingency tables where sample sizes are small. The outcomes of the test were binary: selected genes either belong or do not belong to a specified pathway and were tested for independence with a binary disease phenotype, eg normal or have CD. For CMP, domains of known disease genes were queried from the database and compared to domains of genes in the data set (see Materials and Methods detailed above).

### Validation

SNP and gene density were non-uniform across the genome and gene sizes varied, all of which influenced the number of positional gene candidates analysed. To test for bias due to SNP coverage on Affymetrix chip sets, a validation of a random selection of SNP sets was preformed to check clustering ratios, gene set sizes, and the results of CPS and CMP.

SNP Analysis

### SNP Representation and Distribution

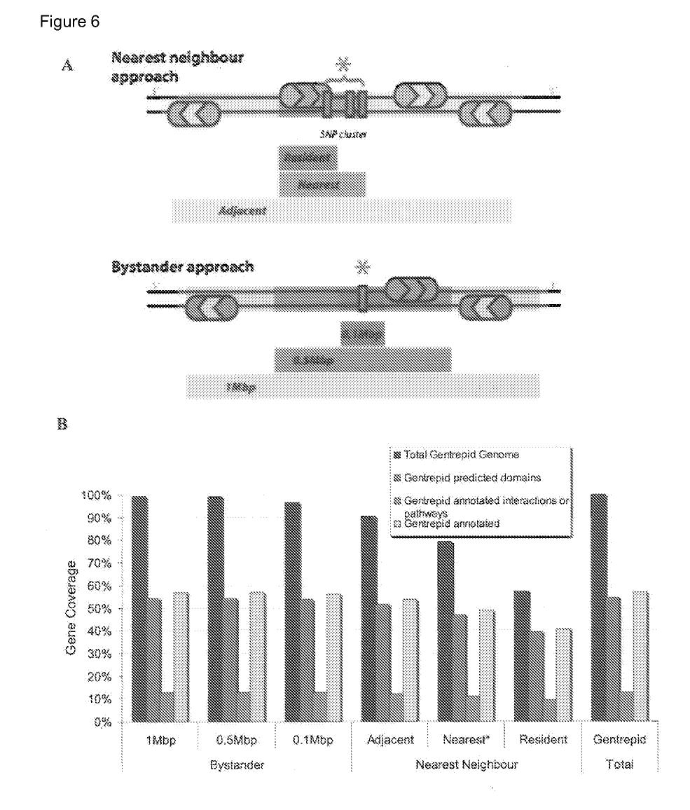

The percentage of genes in the genome covered by SNPs on the Affy500K chip sets under the various SNP to gene mapping assumptions was preformed. The present inventors determined if the genes covered by SNPs on the Affy500K chip sets were represented by associated pathways and domains as determined by the present invention. Genes that were present in RefSeq were defined as “characterized” genes and those that had a predicted domain through either Pfam, or pathways and interactions partners by the present invention were defined as “annotated”. FIG. 6B shows coverage of the human genome by the Affy500K chip sets using the three gene mapping assumptions of each of the NN and BY approaches. When the most common NN assumption was used on the GWAS (nearest NN set), only about 76% of characterized genes were associated with a SNP. The gene coverage increased to about 90% when nearest genes on both strands in both the 3′ and 5′ direction with the SNP (adjacent NN set) were included. When a BY approach was used, gene coverage increased, ranging from about 96 to 99.4% for characterized genes.

Once the genes were successfully associated with SNPs, the question then arose: “How many of these genes may be potentially associated with a phenotype by the present invention?” When the entire genome was considered, only about 57% of characterized genes had annotations provided by the present invention and were thus potentially predictable candidates. Most of the coverage was due to Pfam domains, while pathways cover up to 20% of annotated genes (FIG. 6B).

### SNPs and Disease Phenotypes

SNPs that were associated with phenotypes of interest by GWAS were considered. Table 4 summarizes the number of SNPs above each of the significance thresholds. Significant SNPs show strong clustering, with about 50-60% of significant SNPs around certain loci for each phenotype belonging to a cluster, with an average of about 3 SNPs per cluster. Clustering may be due to haplotype blocks with SNPs in linkage disequilibrium. Following SNP to gene mapping, the search space sets range in size from about 100 to 3000 genes: up to 10% of the genome. The inventors found that gene prediction by the present invention in such large search spaces was computationally feasible. As shown in Table 4, more genes were associated with the phenotype-specific SNPs with the two larger bystander intervals. However, the adjacent NN gene set was usually larger than the corresponding interval of about 0.1 Mbp, often an adjacent genes was located farther than the distance threshold used for the flanking intervals.

TABLE 4
Number of SNPs with significant association test p values and number of associated
annotated genes in CPS and CMP methods.

| | | | | Level | | | |
|---|---|---|---|---|---|---|---|
| | | | | WS | MWS | MHS | HS |
| Disease | | | | p ≦ 1e−3 | p ≦ 1e−4 | p ≦ 1e−5 | p < 5e−7 |
| BD | SNPs | | | 797 | 138 | 23 | 0 |
| | SNPs* | | | 513 | 94 | 10 | 0 |
| | Genes | BY | 1 Mbp | 2484 (4372) | 568 (957) | 46 (76) | 0 |
| | | | 0.5 Mbp | 1370 (2395) | 296 (464) | 26 (43) | 0 |
| | | | 0.1 Mbp | 449 (701) | 87 (125) | 8 (13) | 0 |
| | | NN | Adjacent | 880 (1579) | 182 (312) | 14 (28) | 0 |
| | | | Nearest | 332 (504) | 57 (90) | 6 (8) | 0 |
| | | | Resident | 166 (217) | 33 (40) | 5 (5) | 0 |
| CAD | SNPs | | | 696 | 124 | 38 | 22 |
| | SNPs* | | | 410 | 82 | 21 | 10 |
| | Genes | BY | 1 Mbp | 2253 (3701) | 513 (813) | 90 (138) | 36 (56) |
| | | | 0.5 Mbp | 1210 (1972) | 281 (440) | 49 (79) | 23 (40) |
| | | | 0.1 Mbp | 391 (585) | 79 (120) | 20 (30) | 8 (14) |
| | | NN | Adjacent | 725 (1281) | 161 (291) | 47 (71) | 20 (36) |
| | | | Nearest | 240 (397) | 49 (84) | 16 (22) | 5 (11) |
| | | | Resident | 135 (167) | 28 (34) | 10 (11) | 3 (4) |
| CD | SNPs | | | 1064 | 261 | 102 | 63 |
| | SNPs* | | | 501 | 112 | 23 | 10 |
| | Genes | BY | 1 Mbp | 2643 (4431) | 776 (1252) | 178 (271) | 80 (115) |
| | | | 0.5 Mbp | 1505 (2490) | 451 (700) | 104 (152) | 44 (63) |
| | | | 0.1 Mbp | 522 (768) | 138 (203) | 30 (43) | 12 (20) |
| | | NN | Adjacent | 918 (1576) | 233 (383) | 51 (75) | 24 (34) |
| | | | Nearest | 342 (521) | 86 (121) | 19 (25) | 9 (11) |
| | | | Resident | 190 (235) | 54 (64) | 9 (10) | 5 (5) |
| HT | SNPs | | | 737 | 103 | 5 | 0 |
| | SNPs* | | | 432 | 57 | 5 | 0 |
| | Genes | BY | 1 Mbp | 2024 (3432) | 251 (407) | 18 (36) | 0 |
| | | | 0.5 Mbp | 1160 (1906) | 133 (213) | 10 (19) | 0 |
| | | | 0.1 Mbp | 333 (528) | 42 (60) | 4 (5) | 0 |
| | | NN | Adjacent | 760 (1364) | 110 (200) | 8 (18) | 0 |
| | | | Nearest | 251 (418) | 39 (60) | 3 (5) | 0 |
| | | | Resident | 138 (179) | 22 (28) | 2 (2) | 0 |
| RA | SNPs | | | 699 | 104 | 27 | 11 |
| | SNPs* | | | 429 | 75 | 14 | 5 |
| | Genes | BY | 1 Mbp | 2285 (3777) | 595 (956) | 97 (135) | 38 (51) |
| | | | 0.5 Mbp | 1248 (2040) | 326 (526) | 58 (77) | 21 (26) |
| | | | 0.1 Mbp | 407 (583) | 105 (150) | 18 (26) | 7 (10) |
| | | NN | Adjacent | 778 (1372) | 157 (264) | 28 (41) | 7 (11) |
| | | | Nearest | 271 (432) | 47 (79) | 9 (14) | 2 (5) |
| | | | Resident | 147 (183) | 25 (31) | 5 (7) | 2 (4) |
| T1D | SNPs | | | 966 | 276 | 162 | 92 |
| | SNPs* | | | 442 | 103 | 43 | 24 |
| | Genes | BY | 1 Mbp | 2353 (4032) | 668 (1123) | 320 (465) | 270 (379) |
| T2D | SNPs | | | 671 | 116 | 40 | 16 |
| | SNPs* | | | 401 | 68 | 15 | 2 |
| | Genes | BY | 1 Mbp | 1955 (3384) | 331 (588) | 66 (106) | 7 (11) |
| | | | 0.5 Mbp | 1068 (1846) | 187 (311) | 35 (53) | 3 (5) |
| | | | 0.1 Mbp | 354 (571) | 66 (96) | 14 (20) | 1 (2) |
| | | NN | Adjacent | 725 (1264) | 127 (226) | 27 (46) | 5 (6) |
| | | | Nearest | 254 (396) | 46 (66) | 11 (13) | 1 (2) |
| | | | Resident | 132 (170) | 25 (33) | 6 (7) | 1 (2) |

### Abbreviations

Rows—BD, Bipolar Disorder; CAD, Coronary Artery Disease; CD, Crohn's Disease; HT, Hypertension; RA, Rheumatoid Arthritis; T1D, Type I Diabetes and T2D, Type II Diabetes;

Columns—HS, highly significant; MHS, moderately-high significance MWS: moderately-weak significance WS: weakly significant. SNPs—number of implicated loci; SNPs*-number of clusters based on naïve clustering of SNPs within 50 Kbp of one another; “Genes” cells show the number of associated annotated genes with the number of characterized genes in the genome in parenthesis for each SNP mapping approach

Assessment of GWAS Data

To assess the ability of CPS and CMP to extract positional candidates from weakly significant data, analysis of the GWAS-implicated loci at the different levels of stringency chosen using both the NN and BY mapping assumptions was preformed.

To determine if genes selected by CPS and CMP were true positives, several approaches to assess the results were preformed. Firstly, predictions were compared to random sampling. Secondly, comparisons of the results to genes associated with the HS SNPs by the WTCCC and other meta-analyses where available were preformed.

The ability to extract known disease genes within the search space was also assessed by using CPS and CMP.

### Common Module Profiling Results

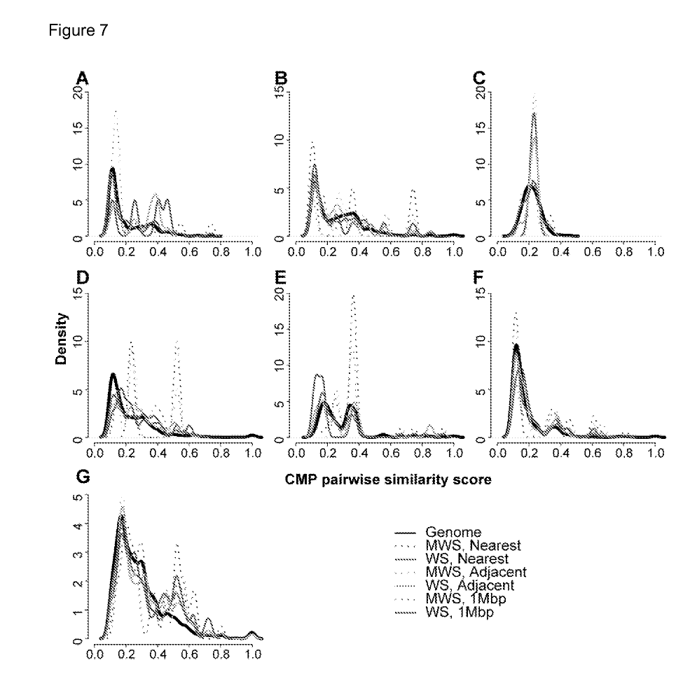

When searching for candidates using known disease gene input mode, CMP assigned a pairwise similarity score between 0 and 1.16. Using a benchmark set suggested by Turner et al (2003), the inventors determined that a pairwise similarity score of 0.4 between a test gene and a known disease gene was a conservative threshold above which a test gene may be considered a candidate. In addition, the present invention allows for known disease genes to be retrieved by CMP using leave-one-out cross validation down to a threshold of 0.1 without the introduction of too much noise. FIG. 7 illustrates a plot of pairwise CMP scores for all genes associated with the seven phenotypes (BD, CAD, CD, HT, RA, T1D and T2D), as well as the genome as a whole. FIG. 7 details genes resembling known disease genes are enriched in the SNP associated regions compared to the genome for most phenotypes. An exception was CD and T1D (FIGS. 7C,F) and may indicate that the known disease genes for these phenotypes are not representative of CD and T1D. Reducing the threshold as far as 0.1 to search for further candidates for CD and T1D may introduce unwanted noise. Using the 0.4 threshold, the number of genes with common domains from the disease associated SNPs is slightly lower than those of a random sample (Table 7).

Using ab initio input mode, the number of predictions by CMP was generally fewer than random for the BY mapping but similar for the NN mappings (Table 7). For instance, using 432 loci from clustered HT SNPs as input and the 1 Mbp BY mapping, CMP ab initio predicts 73 genes with 23 significant domain combinations, while a random sample using similar parameters predicts over 180 genes. But using the adjacent mapping for the same number of loci, CMP ab initio predicts 28 genes using the HT loci and 26 genes using a random sample. The difference in the prediction results between the mappings for the phenotypes and the random samples may be a result of the arbitrary significance thresholds we chose for multidomain proteins (χ2 max_unique>10-5) and single domain proteins (χ2 min>10-2). The upper significance is particularly sensitive when multidomain proteins are implicated in the phenotype. The different mapping approaches may require alternate thresholds. Also, T1D differs from other diseases in this test. Since we are counting the number of possible candidate genes, and not the loci which are used to calculate the significance, certain loci with many genes with common domains such as the HLA and histone loci, inflate the results.

An important difference between genes chosen by random sampling and genes associated with phenotype-related SNPs was that randomly chosen genes contain on average about two or three common domains while phenotype associated genes typically have more than three domains in common

Overall CMP ab initio input mode was more successful in predicting disease genes than in known disease gene input mode, with novel functional implications for the phenotypes.

TABLE 5
WS set. Number of genes and pathways returned by CPS in both known (CPS-k) and
ab initio (CPS-ab) modes for significant pathways (p < 0.05) and for mapped
GWAS SNPs (n) and random SNPs (r).

| | | | | CPS-k | | | | CPS-ab | | | |
|---|---|---|---|---|---|---|---|---|---|---|---|
| | | | | Genes | | Pathways | | Genes | | Pathways | |
| Disease | Approach | Range | Annotated | n | r | n | r | n | r | n | r |
| BD | BY | 1 Mbp | 706 | 0 | 11.19 | 0 | 0.32 | 81 | 162.94 | 11 | 18.61 |
| | | 0.5 Mbp | 389 | 0 | 5.92 | 0 | 0.26 | 29 | 91.46 | 9 | 16.53 |
| | | 0.1 Mbp | 131 | 0 | 4.45 | 0 | 0.48 | 14 | 31.98 | 3 | 11.52 |
| | NN | Adjacent | 254 | 0 | 23.68 | 0 | 1.23 | 53 | 70.42 | 14 | 12.68 |
| | | Nearest | 97 | 0 | 8.18 | 0 | 0.97 | 16 | 31.1 | 4 | 11.11 |
| | | Resident | 51 | 0 | 3.57 | 0 | 0.66 | 21 | 17.38 | 10 | 8.91 |
| CAD | BY | 1 Mbp | 665 | 55 | 29.52 | 3 | 1.71 | 103 | 138.52 | 11 | 18.64 |
| | | 0.5 Mbp | 360 | 4 | 14.63 | 1 | 1.52 | 19 | 75.9 | 5 | 15.95 |
| | | 0.1 Mbp | 119 | 0 | 5.08 | 0 | 1.05 | 23 | 25.69 | 6 | 10.72 |
| | NN | Adjacent | 230 | 4 | 11.21 | 1 | 1.37 | 46 | 56.36 | 8 | 12.28 |
| | | Nearest | 85 | 0 | 5.24 | 0 | 1.19 | 20 | 23.69 | 5 | 9.55 |
| | | Resident | 51 | 0 | 3.26 | 0 | 1.06 | 7 | 13.32 | 2 | 7.06 |
| CD | BY | 1 Mbp | 869 | 65 | 27.16 | 3 | 1.56 | 162 | 163.58 | 13 | 18.88 |
| | | 0.5 Mbp | 501 | 7 | 10.88 | 2 | 1.08 | 43 | 90.81 | 12 | 16.42 |
| | | 0.1 Mbp | 181 | 0 | 1.42 | 0 | 0.4 | 49 | 31.25 | 14 | 11.38 |
| | NN | Adjacent | 316 | 19 | 1.74 | 2 | 0.37 | 82 | 68.98 | 11 | 12.75 |
| | | Nearest | 119 | 15 | 0.41 | 2 | 0.15 | 51 | 29.77 | 15 | 10.84 |
| | | Resident | 69 | 7 | 0.16 | 3 | 0.08 | 17 | 16.91 | 10 | 8.81 |
| HT | BY | 1 Mbp | 602 | 5 | 46.19 | 2 | 2.74 | 77 | 148.03 | 15 | 18.96 |
| | | 0.5 Mbp | 348 | 5 | 23.17 | 2 | 2.23 | 35 | 77.93 | 6 | 15.85 |
| | | 0.1 Mbp | 105 | 9 | 8.25 | 5 | 1.77 | 33 | 26.33 | 23 | 10.84 |
| | NN | Adjacent | 226 | 48 | 23.13 | 4 | 1.85 | 61 | 57.43 | 10 | 11.72 |
| | | Nearest | 68 | 18 | 9.61 | 3 | 1.77 | 29 | 25.2 | 10 | 9.84 |
| | | Resident | 40 | 6 | 4.87 | 1 | 1.34 | 8 | 14.24 | 3 | 7.57 |
| RA | BY | 1 Mbp | 686 | 8 | 45.99 | 1 | 4 | 69 | 148.32 | 8 | 19.03 |
| | | 0.5 Mbp | 386 | 8 | 19.74 | 1 | 2.84 | 40 | 77.16 | 13 | 15.84 |
| | | 0.1 Mbp | 127 | 8 | 3.98 | 4 | 1.17 | 18 | 26.14 | 8 | 10.8 |
| | NN | Adjacent | 235 | 22 | 5.45 | 4 | 0.91 | 65 | 57.17 | 12 | 11.81 |
| | | Nearest | 92 | 10 | 2.42 | 1 | 0.58 | 16 | 25.2 | 5 | 9.83 |
| | | Resident | 55 | 6 | 1.43 | 2 | 0.45 | 11 | 14.15 | 6 | 7.56 |
| T1D | BY | 1 Mbp | 693 | 21 | 44.57 | 3 | 3.06 | 133 | 147.64 | 15 | 18.88 |
| | | 0.5 Mbp | 398 | 19 | 21.75 | 3 | 2.5 | 49 | 80.97 | 13 | 16.08 |
| | | 0.1 Mbp | 131 | 23 | 6.91 | 11 | 1.65 | 44 | 27.02 | 22 | 11.01 |
| | NN | Adjacent | 236 | 18 | 16.44 | 7 | 2.05 | 52 | 60.52 | 18 | 12.29 |
| | | Nearest | 88 | 18 | 7.25 | 9 | 1.83 | 41 | 26.07 | 22 | 10.19 |
| | | Resident | 47 | 8 | 4.28 | 8 | 1.48 | 18 | 14.58 | 21 | 7.7 |
| T2D | BY | 1 Mbp | 558 | 50 | 49.24 | 7 | 4.36 | 110 | 134.64 | 18 | 18.85 |
| | | 0.5 Mbp | 306 | 43 | 24.8 | 10 | 3.33 | 74 | 74.56 | 26 | 15.88 |
| | | 0.1 Mbp | 99 | 7 | 7.15 | 2 | 1.97 | 19 | 25.63 | 7 | 10.81 |
| | NN | Adjacent | 215 | 23 | 12.82 | 5 | 2.26 | 58 | 55.48 | 16 | 12.44 |
| | | Nearest | 78 | 15 | 6.44 | 7 | 1.83 | 28 | 23.26 | 15 | 9.52 |
| | | Resident | 42 | 3 | 4.21 | 1 | 1.56 | 9 | 13.02 | 4 | 7.06 |

Common Pathway Scanning results

In both known disease gene and ab initio mode, the number of genes predicted by CPS for the WS- and MWS-implicated loci was significantly less than if randomly sampled (Table 5).

This was most apparent for the BY mapping using the less stringent p value sets: for instance, 429 loci were used from clustered RA SNPs as input and the 1 Mbp BY mapping, CPS predicts 69 genes in ab initio mode; whereas for a sample of 429 random SNPs mapped in the same way, CPS usually returns over 148 genes. Unexpectedly, the number of significant pathways (Fishers test p<0.05) associated with genes predicted using the GWAS data was not different to random: for the 1 Mbp BY mapping, CPS returned 18 significant pathways for both GWAS SNPs and the random SNPs. However on more careful inspection of the data, it can be clearly seen that the true data has a subset of genes that are clustered into common pathways. This clustering of genes is taken to be in 1 dicative of information gain. Thus the system is extracting relevant pathways but the statistical tests inappropriately rate some of the random data as significant.

The ability of CPS to prioritize WTCCC candidates is shown in Table 5 where predicted genes are assigned an ordinal priority based on their ranking score. Despite being confronted with increasingly large search spaces, CPS is still able to extract biologically relevant genes from the increasingly less significant genetic data. In the MHS and MWS sets, the lowest priority given to a known disease gene as collated from OMIM is 11th in both known and ab initio mode. The mapping approach does not have a noticeable effect on the priority, for instance IL2RA, a risk gene for T1D identified in OMIM, has similar priority for all mapping methods. However, some deterioration of the signal is apparent for the least statistically significant data (WS), when the more demanding ab initio method is employed; or when larger search spaces are used. For example, generally the priority assigned to a particular gene using the 1 Mbp BY mapping is lower than the priority of the adjacent NN mapping approach, suggesting that the signal-to-noise ratio is decreasing.

The ability of CPS to prioritize known disease genes is shown in Table 6. Known disease gene mode is generally a more powerful discovery tool when retrieving novel genes associated with pathways involving disease genes previously linked to the phenotype. If a known disease gene of the implicated pathway is within the search space, the pathway will be equally ranked by both known and ab initio methods, as the same gene will be retrieved by both methods. If a known disease gene of the pathway is outside the search space, the pathway will be ranked higher in known disease gene mode than in ab initio, which has no additional knowledge of the pathway. Thus known disease gene mode generally has a better chance of reaching statistical significance when dealing with a pathway known to be associated with the phenotype. This is the case for CDKN2B in CAD and CHRM3 in HT. Ab initio mode however is superior when a putative novel pathway is hidden in the data, for example genes GCH1 SMARCA5 and ASCC3L1 in the pathway “Folate biosynthesis” in HT. Altered folate and homocysteine metabolism are thought to play a role in the early stages of hypertension, although the exact mechanisms are still unknown.

Overall CPS was more successful in predicting disease genes in the larger search spaces associated with lower significance levels, although some dilution of the signal was apparent for WS data, particularly for more generous mappings. This is partially due to the nature of the method which assigns higher statistical significance to a pathway when many discrete loci are involved. However, it may also reflect the architecture of complex diseases.

TABLE 6
Ability of CPS to prioritize known disease genes in search space from the different
significance sets

| | | | Known | | | | | | Ab initio | | | | | |
|---|---|---|---|---|---|---|---|---|---|---|---|---|---|---|
| | | | MHS | | MWS | | WS | | MHS | | MWS | | WS | |
| Disease | Gene | Mapping | n | p | n | p | n | p | n | p | n | p | n | p |
| BD | — | | | | | | | | | | | | | |
| CAD | CX3CR1 | 1 Mbp | | | 1 | 1^{st} | 1 | 1^{st} | | | 1 | 1^{st} | 1 | 4^{th} |
| | | Adjacent | | | 1 | 2^{nd} | 1 | 3^{rd} | | | 1 | 3^{rd} | 1 | 7^{th} |
| | IRS1 | 1 Mbp | | | 1 | 4^{th} | 1 | 6^{th} | | | 3 | 3^{rd} | 10 | 9^{th} |
| | | Adjacent | | | 1 | 2^{nd} | 1 | 7^{th} | | | 3 | 1^{st} | 9 | 7^{th} |
| | LRP6 | 1 Mbp | | | | | 0 | — | | | | | 1 | 9^{th} |
| | NOS3 | 1 Mbp | | | | | 0 | — | | | | | 11 | 5^{th} |
| | CD36 | 1 Mbp | | | 1 | 4 | 1 | 6 | | | 4 | 3^{rd} | 5 | 10^{th} |
| | | Adjacent | | | 1 | 2 | 1 | 7 | | | 3 | 2^{nd} | 4 | 6^{th} |
| CD | IL23R | 1 Mbp | 2 | 1^{st} | 2 | 1^{st} | 2 | 1^{st} | 2 | 1^{st} | 2 | 2^{nd} | 2 | 4^{th} |
| | | Adjacent | 2 | 1^{st} | 2 | 1^{st} | 2 | 1^{st} | 2 | 1^{st} | 2 | 1^{st} | 2 | 3^{rd} |
| | DLG5 | 1 Mbp | | | | | 0 | — | | | | | 0 | — |
| | | Adjacent | | | | | 0 | — | | | | | 0 | — |
| | CARD15 | 1 Mbp | 0 | — | 0 | — | 0 | — | 0 | — | 0 | — | 0 | — |
| | | Adjacent | 0 | — | 0 | — | 0 | — | 0 | — | 0 | — | 0 | — |
| HT | AGT | 1 Mbp | | | | | 3 | 8^{th} | | | | | 6 | 19^{th} |
| | | Adjacent | | | | | 3 | 9^{th} | | | | | 5 | 11^{th} |
| | AGTR1 | 1 Mbp | | | | | 4 | 1^{st} | | | | | 5 | 1^{st} |
| | EPHX1 | 1 Mbp | | | | | 2 | 9^{th} | | | | | 2 | 21^{st} |
| | PTGIS | 1 Mbp | | | | | 1 | 13^{th} | | | | | 2 | 20^{th} |
| RA | PTPN22 | 1 Mbp | 0 | — | 0 | — | 0 | — | 0 | — | 0 | — | 0 | — |
| | | Adjacent | 0 | — | 0 | — | 0 | — | 0 | — | 0 | — | 0 | — |
| | HLA- | 1 Mbp | 5 | 2^{nd} | 5 | 3^{rd} | 5 | 3^{rd} | 15 | 2^{nd} | 15 | 4^{th} | 15 | 9^{th} |
| | DRB1 | | | | | | | | | | | | | |
| | | Adjacent | 5 | 2^{nd} | 5 | 3^{rd} | 5 | 3^{rd} | 15 | 1^{st} | 15 | 3^{rd} | 15 | 6^{th} |
| | IL10 | 1 Mbp | | | | | 6 | 1^{st} | | | | | 8 | 2^{nd} |
| | | Adjacent | | | | | 6 | 1^{st} | | | | | 7 | 2^{nd} |
| | CIITA | 1 Mbp | | | | | 1 | 7^{th} | | | | | 1 | 19^{th} |
| | NFKBIL1 | 1 Mbp | | | 0 | — | 0 | — | | | 0 | — | 0 | — |
| T1D | CCR5 | 1 Mbp | | | 1 | 1^{st} | 1 | 1^{st} | | | 3 | 2^{nd} | 6 | 3^{rd} |
| | CTLA4 | 1 Mbp | | | 0 | — | 0 | — | | | 3 | 6^{th} | 3 | 11^{th} |
| | | Adjacent | | | 0 | — | 0 | — | | | 3 | 3^{rd} | 3 | 6^{th} |
| | PTPN22 | 1 Mbp | 0 | — | 0 | — | 0 | — | 0 | — | 0 | — | 0 | — |
| | | Adjacent | 0 | — | 0 | — | 0 | — | 0 | — | 0 | — | 0 | — |
| | IL2RA | 1 Mbp | 3 | 1^{st} | 3 | 1^{st} | 3 | 1^{st} | 7 | 1^{st} | 7 | 2^{nd} | 7 | 3^{rd} |
| | | Adjacent | 3 | 1^{st} | 3 | 1^{st} | 3 | 1^{st} | 7 | 1^{st} | 7 | 1^{st} | 7 | 1^{st} |
| | ITPR3 | 1 Mbp | 0 | — | 0 | — | 0 | — | 7 | 1^{st} | 7 | 6^{th} | 7 | 6^{th} |
| | | Adjacent | 0 | — | 0 | — | 0 | — | 7 | 1^{st} | 7 | 5^{th} | 7 | 7^{th} |
| | OAS1 | 1 Mbp | 0 | — | 0 | — | 0 | — | 0 | — | 0 | — | 0 | — |
| T2D | TCF7L2 | 1 Mbp | 6 | 3^{rd} | 6 | 3^{rd} | 6 | 6^{th} | 4 | 1^{st} | 7 | 2^{nd} | 9 | 6^{th} |
| | | Adjacent | 6 | 3^{rd} | 6 | 4^{th} | 6 | 5^{th} | 0 | — | 2 | 3^{rd} | 9 | 2^{nd} |
| | TCF2 | 1 Mbp | | | | | 1 | 12^{th} | | | | | 1 | 21st |
| | AKT2 | 1 Mbp | | | | | 9 | 1^{st} | | | | | 25 | 1^{st} |
| | CDKAL1 | 1 Mbp | 0 | — | 0 | — | 0 | — | 0 | — | 0 | — | 0 | — |
| | | Adjacent | 0 | — | 0 | — | 0 | — | 0 | — | 0 | — | 0 | — |
| | WFS1 | 1 Mbp | | | | | 0 | — | | | | | 0 | — |

n - number of pathways gene has in common with either known disease genes (known mode) or other genes in the set (ab initio mode)
p - priority given to gene in CPS based on the highest rank of the most common pathway

TABLE 7
WS set. Number of genes returned by CMP in both known (CMP-k) and ab initio (CMP-
ab) mode and the number of common domain combinations.

| | | | | CMP-k | | | | CMP-ab | | | |
|---|---|---|---|---|---|---|---|---|---|---|---|
| | | | | Genes | | Domains | | Genes | | Domains | |
| Disease | Approach | Range | Annotated | n | r | n | r | n | r | n | r |
| BD | BY | 1 Mbp | 2374 | 18 | 21.3 | 3 | 4.52 | 48 | 233.34 | 13 | 23.63 |
| | | 0.5 Mbp | 1314 | 11 | 12.4 | 3 | 3.56 | 27 | 102.42 | 8 | 16.33 |
| | | 0.1 Mbp | 431 | 3 | 4.34 | 2 | 1.77 | 14 | 22.77 | 5 | 7.97 |
| | NN | Adjacent | 845 | 11 | 10.82 | 3 | 3.28 | 42 | 33.44 | 15 | 12.52 |
| | | Nearest | 320 | 3 | 4.1 | 3 | 1.77 | 7 | 14.44 | 4 | 5.68 |
| | | Resident | 162 | 1 | 1.61 | 1 | 0.71 | 10 | 13.31 | 2 | 4.94 |
| CAD | BY | 1 Mbp | 2179 | 38 | 46.27 | 9 | 10.23 | 47 | 179.79 | 14 | 21.53 |
| | | 0.5 Mbp | 1171 | 21 | 28.06 | 8 | 7.84 | 31 | 81.02 | 11 | 15.05 |
| | | 0.1 Mbp | 386 | 8 | 10.86 | 6 | 4.19 | 12 | 18.75 | 6 | 6.63 |
| | NN | Adjacent | 706 | 18 | 20.55 | 8 | 6.96 | 24 | 25.25 | 10 | 9.98 |
| | | Nearest | 235 | 6 | 9.03 | 5 | 3.75 | 11 | 10.45 | 6 | 4.27 |
| | | Resident | 133 | 4 | 5.83 | 4 | 2.49 | 11 | 10.04 | 4 | 3.93 |
| CD | BY | 1 Mbp | 2535 | 6 | 8.27 | 2 | 2.31 | 66 | 225.52 | 21 | 23.24 |
| | | 0.5 Mbp | 1445 | 1 | 5.09 | 1 | 1.76 | 52 | 98.74 | 19 | 16.17 |
| | | 0.1 Mbp | 497 | 0 | 2.51 | 0 | 1.12 | 22 | 22.38 | 10 | 7.73 |
| | NN | Adjacent | 875 | 1 | 3.81 | 1 | 1.39 | 41 | 32.13 | 14 | 12.27 |
| | | Nearest | 324 | 0 | 1.88 | 0 | 0.88 | 11 | 13.5 | 5 | 5.35 |
| | | Resident | 180 | 0 | 1.57 | 0 | 0.74 | 6 | 12.76 | 3 | 4.76 |
| HT | BY | 1 Mbp | 1952 | 70 | 72.63 | 8 | 11.75 | 73 | 186.97 | 23 | 21.91 |
| | | 0.5 Mbp | 1123 | 41 | 42.58 | 7 | 9.11 | 28 | 84.22 | 12 | 15.36 |
| | | 0.1 Mbp | 329 | 11 | 16.05 | 3 | 5.27 | 4 | 19.13 | 2 | 6.79 |
| | NN | Adjacent | 735 | 30 | 34.82 | 6 | 8.84 | 28 | 26.91 | 13 | 10.58 |
| | | Nearest | 243 | 6 | 13.93 | 2 | 4.89 | 10 | 11.48 | 5 | 4.64 |
| | | Resident | 135 | 3 | 9.34 | 2 | 3.57 | 4 | 10.37 | 2 | 4.01 |
| RA | BY | 1 Mbp | 2185 | 17 | 13.31 | 4 | 3.55 | 41 | 186.18 | 12 | 21.9 |
| | | 0.5 Mbp | 1203 | 8 | 8.57 | 3 | 2.85 | 31 | 84.23 | 9 | 15.33 |
| | | 0.1 Mbp | 397 | 2 | 3.68 | 1 | 1.55 | 10 | 19.22 | 5 | 6.78 |
| | NN | Adjacent | 752 | 6 | 6.14 | 3 | 2.17 | 17 | 26.9 | 9 | 10.51 |
| | | Nearest | 263 | 1 | 2.68 | 1 | 1.15 | 13 | 11.36 | 5 | 4.61 |
| | | Resident | 143 | 1 | 1.9 | 1 | 0.82 | 18 | 10.24 | 7 | 3.98 |
| T1D | BY | 1 Mbp | 2225 | 23 | 19.67 | 3 | 4.1 | 70 | 192.67 | 18 | 22.16 |
| | | 0.5 Mbp | 1295 | 17 | 12.21 | 3 | 3.52 | 29 | 87.61 | 8 | 15.47 |
| | | 0.1 Mbp | 509 | 8 | 5.35 | 3 | 2.14 | 15 | 19.52 | 6 | 6.93 |
| | NN | Adjacent | 800 | 11 | 10.86 | 3 | 3.46 | 21 | 27.56 | 9 | 10.81 |
| | | Nearest | 299 | 6 | 4.6 | 3 | 1.97 | 15 | 11.61 | 6 | 4.7 |
| | | Resident | 173 | 3 | 3.11 | 1 | 1.39 | 8 | 10.56 | 3 | 4.06 |
| T2D | BY | 1 Mbp | 1862 | 82 | 107.68 | 19 | 19.03 | 58 | 172.47 | 14 | 21.25 |
| | | 0.5 Mbp | 1026 | 45 | 63.84 | 16 | 15.23 | 17 | 78.53 | 4 | 15.06 |
| | | 0.1 Mbp | 338 | 21 | 26.02 | 11 | 9.28 | 8 | 18.1 | 4 | 6.4 |
| | NN | Adjacent | 698 | 48 | 52.94 | 15 | 14.01 | 15 | 24.61 | 5 | 9.68 |
| | | Nearest | 241 | 20 | 24.18 | 12 | 8.96 | 9 | 10.26 | 4 | 4.11 |
| | | Resident | 129 | 11 | 14.8 | 8 | 6.25 | 11 | 9.75 | 6 | 3.88 |

### Results for Specific Phenotypes

### Bipolar Disorder (BD)

CPS did not predict any genes using known disease gene input mode but up to 81 genes in ab initio input mode (Table 5). For known disease gene input mode, CMP predicted up to 18 genes. In ab initio input mode, the number of predictions reaching the arbitrary threshold χ2 max_unique was at most about 48 genes (Table 7). Predominant molecular processes of the CMPab predictions for the BD phenotype were transcriptional activation and neurotransmitter-gated channels.

None of the known disease genes were in any of the search spaces mapped from the SNPs. The present inventors further investigated the ability of the method of the present invention to predict novel implications from the WTCCC data from the highly significant SNPs of the WTCCC data. The strongest signal (p=6.3×10⁻⁸) was near three genes of possible significance: PALB2, NDUFAB1 and DCTN5. Of these, CPS ab initio input more predicted NADH dehydrogenase NDUFAB1 to be a relevant gene as part of the oxidative phosphorylation pathway but the result was not statistically significant (p=0.77). The GABA neurotransmitter receptor, GABRB1, near an associated region (p=6.2×10⁻⁵), was predicted by CPS with the known disease gene HTR2A, a serotonin receptor, as both genes are part of the “Neuroactive ligand-receptor interaction” pathway, but the result did not reach statistical significance in any of the mappings (p=0.507). GABRB1 was also predicted in CMP ab initio input mode as the highest scoring prediction using the MWS data for the adjacent mapping along with GABRA4. GABA receptors have been previously associated with BD and schizophrenia.

No significant predictions were made by CPS in known disease mode (table 8). In CPS ab initio input mode, the top ranking and most significant pathway of the nearest mapping approach for 1 WS set was the “Leukocyte transendothelial migration” pathway (p=2 0.003). This pathway was also significant and top ranking using the adjacent mapping for the WS set (Table 8). Leukocyte migration was a critical in immune surveillance and inflammation. Calcium homeostasis and immune system imbalance were implicated in other brain disorders such as schizophrenia: MYL12B is differentially expressed in patients compared to controls (Table 8). Recent studies suggest bipolar patients have similar immune profiles to schizophrenic patients, specifically in endothelium-related inflammation processes. Two other significant pathways using the nearest mapping were the “Heparan sulfate biosynthesis” and “Synaptic Proteins at the Synaptic Junction” pathways (p=0.007), which were both notable (Table 8). The heparan sulfate biosynthesis pathway was implicated in the study by Torikami et al (Torkamani, A., Topol, E. J., and Schork, N. J. (2008) Pathway analysis of seven common diseases assessed by genome-wide association. Genomics 92, 265-272). Sulfotransferases NDST3, HS6ST1 and HS3ST1 are expressed in the brain, inactivate dopamine through sulfation; defects in sulfotransferase activity have been linked to bipolar disorder. The synaptic proteins implicated CPS are also known to be involved in various brain disorders. NRXN3 neurexin 3, a neuronal cell surface protein that may be involved in cell recognition and cell adhesion and predominately expressed in the brain, has been associated with addiction and reward behaviour and also recently implicated in obesity. ANK3, ankyrin G, is an adaptor protein found at axon initial segments that has been shown to regulate the assembly of voltage-gated sodium channels and was associated with bipolar disorder in recent GWAS.74; 75 DLG2 also known as PSD-95, interacts with N-methyl-D-Aspartate (NMDA) receptors. Abnormal expression of the NMDA receptors and its interacting molecules of the postsynaptic density (PSD) may be involved in the pathophysiology of schizophrenia. Increased transcript expression was associated with decreased protein expression, suggesting abnormal translation 1 and/or accelerated protein degradation of these molecules in schizophrenia. The adjacent and BY mappings implicated pathways involved in signal transduction and signaling molecules, with “Neuroactive ligand4 receptor interaction” featuring prominently. None of the top ranking pathways were significant in the 1 MBp BY mapping, but the most significant pathway was the “Antigen processing and presentation” (p=0.0005) containing KIR2D genes, PSME1 and PSME2, and CALR, again implicating an immune impairment. The KIR2D genes are known to be polymorphic and are clustered within 1 Mbp.

Of the few predictions made by CMP using known disease genes as seeds, several were neurotransmitter transporters (Table 8). The highest scoring prediction (0.741) was SLC6A2 with the known disease gene SLC6A3, a neurotransmitter that transports dopamine. SLC6A2 transports noradrenalin. Also implicated were SLC6A11 (0.462) and SLC6A1 (0.502), both of which transport GABA. Another gene of interest is TMTC3 (0.405), which has a TRP_{—}1 (PF00515) domain like the known disease gene FKBP5, an immunophilin.

Several CMP ab initio predictions involve glutaminergic neurotransmission, underactivity of which has been proposed to underlie the pathophysiology of several major mental illnesses. The major glutamate receptors were the NMDA receptors which are not implicated directly, but indirectly through their interactors, DLG2, MPP6 and MAGI1. DLG2 was independently predicted by CPS ab initio in the “Synaptic Proteins at the Synaptic Junction” pathway. Other predicted glutamate receptors are the ionotropic glutamate receptors GRIK1 and GRIK2. Genes of this family have previously been associated with bipolar and other mental illnesses. A chromosome abnormality disrupting the kainate class ionotropic glutamate receptor gene, GRIK4/KA1, in an individual with schizophrenia and learning disability (mental retardation) was previously described. GRIK3 copy number variations have been reported in post-mortem studies of bipolar patients. Underexpression of GRIK2 has previously associated with bipolar in post mortem studies. The involvement of synaptic vesicles predicted by CPS is independently supported by different genes predicted by CMP ab initio: SH3GL2 and SH3GL3. Disruption of the ubiquitin proteasome system has recently been implicated in schizophrenia and bipolar disorder. Many kelch-repeat proteins are involved in organization of the cytoskeleton via interaction with actin and intermediate filaments, whereas BTB domains have multiple cellular roles, including recruitment to E3 ubiquitin ligase complexes. The identification of the BACK domain in BTB and kelch proteins, and its high conservation across metazoan genomes, suggest an important function for this domain with a possible role in substrate orientation in Cullin3-based E3 ligase complexes. Eicosapentaenoic acid supplementation provided improvement in schizophrenia patients, while the combination of (eicosapentaenoic acid+docosahexaenoic acid) provided benefit in bipolar disorders. The LDL-like receptors may be relevant. ETS factors are trans-acting phosphoproteins that have key roles in cell migration, proliferation, differentiation and oncogenic transformation. Translocation of ETS transcription factors occurs in multiple cancers including prostate, Ewing's sarcoma and prostate cancer and leukemia. ITIH genes are involved in the acute phase response and hyaluronan metabolic process. Two glycosyltransferases, EXT1 and EXTL1, likely to be involved in GAG synthesis are also implicated. Serum acid glycosaminoglycans (GAG) levels were measured in 50 normals and 177 samples from different types of psychiatric patients. Mean levels were significantly higher in paranoid type schizophrenia, organic brain syndrome associated psychosis and manic type manic depressive psychosis. The acute phase response may also be relevant to lipid metabolism. KCNN3 and KCNN4 are small conductance Ca2+-activated potassium channels. CAG triplet expansions associated with KCNN3 have been found in some kindreds with schizophrenia or bipolar disorder I⁸⁶ but not in others. KCNN4 has not previously been implicated.

Novel CMP ab initio input mode predictions involve post-translational modification of amino acids and dysfunction of metabolism. The PADI genes are peptidyl-arginine deiminases that regulate gene expression via post-translational citrullination of arginine residues in histones, but may also act on other protein substrates. The PADI genes have previously been associated with rheumatoid arthritis and citrnullation of various proteins has been demonstrated in multiple sclerosis, which can be associated with mood disorders including bipolar, as well as a several brain disorders including a murine model of autoimmune encephalitis and Alzheimer's disease patients. The prediction of nuclear hormone receptors as well as catabolic mitochrondrial enzymes implicate dysfunction of metabolism in bipolar disorder. Several nuclear hormone receptors predicted by CMP ab initio input mode in bipolar are supported (Table 8). Defects in one of these, THRB, are the cause of generalized and pituitary thyroid hormone resistance (MIM 188570, 274300 and 145650 respectively). Many of the limbic system structures where thyroid hormone receptors are prevalent have been implicated in the pathogenesis of mood disorders. The influence of the thyroid system on neurotransmitters (particularly serotonin and norepinephrine), which putatively play a major role in the regulation of mood and behavior, may contribute to the mechanisms of mood modulation. Two other hormone receptors, the androgenic nuclear hormone receptors ESR1 and ESRRG, are implicated along with their binding partners: ESRR1 binds TLE1, a transducin-like corepressor, MLL2, a histone lysine methylase forms a complex with the estrogen receptor ESR1.91 A fourth nuclear hormone receptor, NR2F2, is specifically implicated in regulation of apolipoprotein A-I gene transcription. Altered lipid metabolism has been implicated in brain injury and disorders. The mitochrondrial enzymes implicated were ACAD8, IVD and GCDH. IVD and ACAD8 catabolise branched chain amino acids, which are toxic in excess, and were also predicted candidates for T2D and CAD. GCDH, which was predicted only for bipolar catabolises lysine and tryptophan. Serotonin (5-HT), which was involved in the pathogenesis and treatment of affective disorders, is synthesized from tryptophan. A CNS regeneration theme was suggested by the semaphorins which control synaptogenesis, axon pruning, and the density and maturation of dendritic spines. Semaphorins and their downstream signaling components regulate synaptic physiology and neuronal excitability in the mature hippocampus, and these proteins were also implicated in a number of developmental, psychiatric, and neurodegenerative disorders. Sem5* associate with chondroitin sulfate proteoglycans (CSPGs) and heparin sulphate proteoglycans.

TABLE 8
Top BD predictions made by CPS and CMP

| | | Mapping | | | | | | |
|---|---|---|---|---|---|---|---|---|
| | | Approach | | | Biological | Genetic | | |
| Group | Method | 1M | Adj | N | Support | Support | Genes | Loci |
| Leukocyte | CPSab | | ✓ | ✓ | ♦♦♦♦ | ▪ | ARHGAP5 | 14q12e |
| transendothelial | | | ✓ | ✓ | ♦♦♦♦ | ▪ | CDH5 | 16q21e |
| migration | | | ✓ | ✓ | ♦♦♦♦ | ▪ | CTNNA2 | 2p12e-p12d |
| | | ✓ | ✓ | ✓ | ♦♦♦♦ | ▪▪ | MMP2 | 16q12.2c |
| | | | ✓ | ✓ | ♦♦♦♦ | ▪ | PTK2 | 8q24.3c |
| | | | ✓ | ✓ | ♦♦♦♦ | ▪ | RAPGEF4 | 2q31.1e |
| | | | ✓ | ✓ | ♦♦♦♦ | ▪▪ | JAM3 | 11q25d |
| | | | ✓ | ✓ | ♦♦♦♦ | ▪ | MYL12B | 18p11.31e |
| | | | ✓ | | ♦♦♦♦ | ▪ | PIK3CG | 7q22.3a-q22.3b |
| | | | ✓ | | ♦♦♦♦ | ▪ | PIK3R1 | 5q13.1c |
| | | | ✓ | | ♦♦♦♦ | ▪ | VAV3 | 1p13.3d-p13.3c |
| | | | ✓ | | ♦♦♦♦ | ▪ | CLDN23 | 8p23.1d |
| | | ✓ | | | ♦♦ | ▪ | NCF4 | 22q12.3d |
| | | ✓ | | | ♦♦ | ▪▪ | RAC2 | 22q13.1a |
| | | ✓ | | | ♦♦ | ▪▪ | ESAM | 11q24.2a |
| Heparan sulfate | CPSab | | ✓ | ✓ | ♦♦♦♦ | ▪ | EXTL1 | 1p36.11b |
| biosynthesis | | | ✓ | ✓ | ♦♦♦♦ | ▪ | NDST3 | 4q26e |
| | | | ✓ | ✓ | ♦♦♦♦ | ▪ | HS6ST1 | 4p15.33e |
| | | | ✓ | | ♦ | ▪ | HS3ST1 | 2q14.3e |
| | | | ✓ | | ♦ | ▪ | EXT1 | 8q24.11b |
| Synaptic Proteins | CPSab | | | ✓ | ♦ | ▪ | ANK3 | 10q21.2a |
| at the Synaptic | | | | ✓ | ♦ | ▪ | DLG2 | 11q14.1d-q14.1e |
| Junction | | | | ✓ | ♦ | ▪ | NRXN3 | 14q24.3d-q31.1a |
| Neurotransmitter | CMPk | ✓ | | | | ▪ | SLC6A1 | 3p25.3a |
| transporters | | ✓ | ✓ | ✓ | | ▪ | SLC6A11 | 3p25.3a |
| | | ✓ | | | | ▪▪ | SLC6A2 | 16q12.2c |
| TPR-containing | CMPk | ✓ | ✓ | ✓ | | ▪ | TMTC3 | 12q21.32a |
| protein | | | | | | | | |
| Kelch-like | CMPab | | ✓ | | ▪▪ | ▪ | KLHL1 | 13q21.33b |
| proteins | | | ✓ | | ▪▪ | ▪ | KLHL25 | 15q25.3b |
| | | | ✓ | | ▪▪ | ▪ | KLHL29 | 2p24.1a |
| | | | ✓ | | ▪▪ | ▪ | KLHL32 | 6q16.1f |
| PADI homologs | CMPab | ✓ | | | ▪▪▪▪* | ▪ | PADI1 &/or | 1p36.13e |
| | | ✓ | | | ▪▪▪▪* | ▪ | PADI2 &/or | 1p36.13e |
| | | ✓ | | | ▪▪▪▪* | ▪ | PADI3 | 1p36.13e |
| | | ✓ | | | ▪▪▪▪* | ▪ | PADI4 &/or | 1p36.13d |
| | | ✓ | | | ▪▪▪▪* | ▪ | PADI6 | 1p36.13d |
| ITIH homologs | CMPab | ✓ | ✓ | | ▪▪▪▪ | ▪ | ITIH1 &/or | 3p21.1c |
| | | ✓ | ✓ | ✓ | ▪▪▪▪ | ▪ | ITIH3 &/or | 3p21.1c |
| | | ✓ | ✓ | | ▪▪▪▪ | ▪ | ITIH4 | 3p21.1c |
| | | ✓ | ✓ | ✓ | ▪▪▪▪ | ▪ | ITIH2 | 10p14e-p14d |
| | | ✓ | ✓ | | ▪▪▪▪ | ▪ | ITIH5 | 10p14e |
| Ca²⁺ -activated K | CMPab | ✓ | | | ▪▪▪▪ | ▪ | KCNN3 | 1q21e3e |
| channels | | ✓ | | | ▪▪▪▪ | ▪ | KCNN4 | 19q13.31b |
| Nuclear factor | CMPab | ✓ | | | ▪ | ▪▪ | NFIX | 19p13.13c-p13.13b |
| transcription | | ✓ | | | ▪ | ▪▪ | NFIA | 1p31.3d |
| factors | | | | | | | | |
| Nuclear hormone | CMPab | | ✓ | | ▪▪▪ | ▪ | NR2F1 | 5q15a |
| transcription | | | ✓ | | ▪▪▪ | ▪ | NR2F2 | 15q26.2c |
| factors | | | ✓ | | □□□ | ▪▪ | ESR1 | 6q25.1c |
| | | | ✓ | ✓ | □□□□ | ▪▪ | ESRRG | 1q41b |
| | | | | ✓ | □□□□ | ▪ | THRB | 3p24.2b |
| | | | | ✓ | □□□□ | ▪ | RXRG | 1q23.3e |
| Transcriptional | CMPab | | ✓ | | ▪ | ▪ | MLL2 | 12q13.12a-q13.12b |
| co-activator | | | ✓ | | ▪ | ▪ | TBRG1 | 11q24.2a |
| Transcriptional | CMPab | | | ✓ | □□□□ | ▪ | TLE1 | 9q21.31d-q21.32a |
| co-repression | | | | ✓ | □□□□ | ▪ | TLE4 | 9q21.31b |
| Kreuppel Zn | CMPab | | | ✓ | □□□ | ▪▪ | ZNF225 | 19q13.31b |
| finger | | | | ✓ | □□□ | ▪▪ | ZNF274 | 19q13.43c |
| transcription | | | | ✓ | □□□ | ▪▪ | ZNF490 | 19p13.2b |
| factors | | | | | | | | |
| ETS transcription | CMPab | | ✓ | | ▪ | ▪ | ETS2 | 21q22.2a |
| factors | | | ✓ | | ▪ | ▪ | ETV6 | 12p13.2b-p13.2a |
| | | | ✓ | | ▪ | ▪ | FLI1 | 11q24.3a |
| | | | ✓ | | ▪ | ▪ | GABPA | 21q21.3a |
| LDL-like | CMPab | | ✓ | | ▪▪ | ▪ | LRP1B | 2q22.1d-q22.2a |
| receptors | | | ✓ | | ▪▪ | ▪ | LRP6 | 12p13.2a |
| Ionotropic | CMPab | | | ✓ | □□□□ | ▪ | GRIK1 | 21q21.3c |
| glutamate | | | | ✓ | □□□□ | ▪ | GRIK2 | 6q16.3c |
| receptors | | | | | | | | |
| GABA receptor | CMPab | | ✓ | ✓ | □□□□ | ▪▪ | GABRA4 | 4p12b |
| subunits | | | ✓ | ✓ | □□□□ | ▪▪ | GABRB1 | 4p12b |
| | | | ✓ | | □□□□ | ▪▪ | GABRB2 | 5q34a |
| NMDA receptor | CMPab | | | ✓ | □□□□ | ▪ | DLG2 | 11q14.1d-q14.1e |
| interactors | | | | | □□□□ | ▪ | MPP6 | 7p15.3a |
| | | | | ✓ | | ▪ | MAGI1 | 3p14.1d-p14.1c |
| collagens | CMPab | | ✓ | ✓ | ▪▪▪ | ▪ | COL5A1 | 9q34.3a |
| | | | ✓ | ✓ | ▪▪▪ | ▪ | COL11A1 | 1p21.1d-p21.1c |
| Receptor Tyr | CMPab | | ✓ | | ▪▪▪ | ▪ | ERBB4 | 2q34c-q34e |
| protein kinase | | | ✓ | | ▪▪▪ | ▪ | IGF1R | 15q26.3 |
| Centromere | CMPab | | ✓ | | ▪▪▪▪ | ▪ | CENPB | 20p13b |
| binding proteins | | | ✓ | | ▪▪▪▪ | ▪ | TIGD2 | 4q22.1c |
| G-coupled | CMPab | | ✓ | | ▪▪▪▪* | ▪ | PIK3CG | 7q22.3a-q22.3b |
| receptor | | | ✓ | | ▪▪▪▪* | ▪ | PIK3C2G | 12p12.3b |
| activation | | | | | | | | |
| semaphorins | CMPab | | | ✓ | □□□□ | ▪ | SEMA5A | 5p15.2d |
| | | | | ✓ | □□□□ | ▪ | SEMA6D | 15q21.1c |
| Glycosyltransferases | CMPab | ✓ | ✓ | | ▪ | ▪ | EXT1 | 8q24.11b |
| | | ✓ | ✓ | | ▪ | ▪ | EXTL1 | 1p36.11b |
| Mitochondrial | CMPab | ✓ | | | ▪▪▪▪ | ▪ | GCDH | 19p13.13c |
| amino acid | | ✓ | | | ▪▪▪▪ | ▪ | IVD | 15q15.1a |
| catabolism | | ✓ | | | ▪▪▪▪ | ▪ | ACAD8 | 11q25e |
| TPR-containing | CMPab | | ✓ | | ▪▪▪ | ▪ | TMTC1 | 12p11.22a |
| proteins | | | ✓ | | ▪▪▪ | ▪ | TMTC3 | 12q21.32a |
| Synaptic vesicle | CMPab | | | ✓ | □□□ | ▪ | SH3GL2 | 9p22.2a |
| exo/endocytosis | | | | ✓ | □□□ | ▪ | SH3GL3 | 15q25.2b |

Bolded genes are predicted independently by more than one method. Loci in bold have previously been associated with the disease.
Abbreviations.
Method: CMPab - CMP ab initio, CMPk - CMP known mode, CPSab - CPS ab initio, CPSk - CPS known mode.
Genetic support: HS ▪▪▪▪, MHS - ▪▪▪, MWS - ▪▪, WS - ▪.
Key to biological support (the present invention's scores): CMPab: ▪▪▪▪* - log χ² ≧ 9, ▪▪▪▪ - 8 ≦ log χ² < 9, ▪▪▪ - 7 ≦ log χ² < 8, ▪▪ - 6 ≦ log χ² < 7, ▪ - 5 ≦ log χ² < 6.
Lower χ² values considered for more genetically significant data based on statistics (≧ MWS) or proximity: □□□□ - 4 ≦ log χ² < 5, □□□ - 3 ≦ log χ² < 4.
Lower χ² values considered for single domain proteins ▴ - log χ² > 2.
CMPk: - Sc > 0.7, - Sc > 0.6, - Sc > 0.5, - Sc > 0.4, ∘ - Sc > 0.25.
CPS: ♦♦♦♦ - p < 0.05 and Top 5, ♦♦♦ - p < 0.05 and Top 10, ♦♦ - Top 5, ♦ - p < 0.05

### Coronary Artery Disease (CAD)

For the CAD phenotype, CPS predicted up to 55 genes using known disease gene input mode; and up to 103 genes in ab initio input mode. The number of significant pathways varied depending on the mapping assumptions, with at most 12 common pathways reaching significance in ab initio input mode (Table 5). For known disease gene input mode, CMP predicted up to 48 genes. In ab initio input mode, the number of predictions was at most 1521, with up to 47 genes reaching the arbitrary threshold χ² max_unique (Table 7).

The present inventors investigated how well the present invention was able to find known disease genes in the search space. This was done using leave-one-out cross validation with known disease genes input mode, as well as in ab initio input mode. The set of 13 known disease genes involved in coronary artery disease collated from OMIM41 related to metabolism, transport and signaling of low-density lipoproteins (LDL). For instance, the genes chemokine (C-X3-C motif) receptor 1, CX3CR1, and chemokine (C-C motif) ligand 2, CCL2, are involved in LDL signaling pathways. The thrombospondin receptor, CD36, and insulin receptor substrate 1, IRS1, are both receptors in the adipocytokine signaling pathway. Of the 13 known disease genes collated from OMIM up to six were associated with CAD SNPs depending on the SNP mapping method employed, and five were detected by CPS (Table 6).

The present inventors investigated the ability of the present invention to predict genes implicated by noted regions associated with the CAD phenotype from the highly significant SNPs from the WTCCC data. The first and most powerful association was on chromosome 9p21.3 (p=1.8×10-14), where two cyclin-dependent kinases inhibitors (CDKN2A/B) and an enzyme involved in polyamine metabolismmethylthioadenosine phosphorylase (MTAP) are located. CPS using the known disease gene input mode predicted one gene (CDKN2B) associated with the WTCCC significant SNPs. CDKN2B is in the common pathway “Small cell lung cancer”. This pathway is top ranking and significant in the nearest NN mapping. CDKN2B may play a role in atherosclerosis through the TGF-β signaling system. A secondary region with modest association (p=1.1×10-4) contained the ADAMTS7 gene, a disintegrin and metalloproteinase with thrombospondin motif. CMP ab initio input mode predicted ADAMTS7 along with other metalloproteases as significant genes in the NN mappings. MTHFD1L, a methlenetetrahydrofolate dehydrogenase (NADP+ dependent) was also implicated by modest association (p=6.3×10-6). CPS ab initio input mode predicted MTHFD1L using the “One carbon pool by folate” and “Glyoxylate and dicarboxylate metabolism” pathways, but neither were top ranking.

The present inventors explored novel predictions by CPS and CMP (Table 9) and the alternate mapping approaches. In known disease gene input mode, top ranking CPS pathway predictions vary between sets and the mapping approach used. The top ranking pathway for the nearest SNP mapping assumption and the HS set currently employed in most GWAS was the “Small cell lung cancer” pathway (Fishers test p=0.039). Increasing the significance cutoff for the SNPs to the MHS set yields the same result, but was no longer statistically significant (p=0.076). For the MWS and WS sets, the top ranking pathway was the “insulin signaling pathway”, but was only significant in the MWS set (p=0.007). However, other mappings of the SNPs were more successful. The top ranking pathways using the adjacent NN mapping that were significant (Fishers test p<0.05) for “Type II diabetes mellitus”, “insulin signaling” and “adipocytokine signaling” pathways in the MWS set. “Actions of Nitric Oxide in the Heart” was the only significant pathway in the WS set for the adjacent mapping. Using the BY mapping approach, the top ranking pathways implicated were involved in environmental information processing and signal transduction across all significance sets, with “Type II diabetes” the most significant pathway. Type II diabetes is a known risk in CAD patients. The possible commonality of pathways underlying CAD and T2D has been demonstrated previously.

In CPS ab initio input mode, the statistically enriched pathways in the individual gene sets were diverse. As in known disease gene input mode, most were involved in cell signaling, environmental information processing and cellular processes. However, the system was sensitive to the alternate mappings and significance thresholds, with the different sets implicating different pathways. Under the usual SNP mapping assumption, the nearest approach implicates genes involved in “SNARE interactions in vesicular transport”, “axon guidance”, and “cell communication”. The adjacent mapping approach implicated pathways similar to the BY mappings, with the “Neuroactive ligand receptor” pathway the most significant top ranking pathway (p=0.049). Using the BY mapping approach, the top ranking pathways implicated are cell signaling and environmental information processing pathways in the WS set, with “MAPK signaling” and “Regulation of the actin cytoskeleton” pathways ranking first, but the only significant top ranking result was “Cytokine-cytokine receptor interaction” (p=0.017). In the MWS set, the top ranking pathways implicated are involved in cellular communication and cell motility while the MHS set implicated cellular processes and cell signaling. Neither sets had results that reached significance.

Several novel candidates are suggested by CMP in known disease gene input mode (Table 9 and Table 10). The predicted genes with the highest similarity to known disease genes were PLG and LPAL2. CMP found seven genes with similarities to LRP6 in the mapped regions, and two matrix metalloproteinases candidates (MMP15, MMP19) similar to MMP3 involved in ECM breakdown. In the 1 Mbp BY mapping approach, genes CCR8, C-C motif chemokine receptor 8, and IRS2, insulin receptor substrate 2, have both good genetic and biological support. CCR8 gene encodes a thymus-specific member of the beta chemokine receptor family, a family of G11 coupled receptors. Chemokines induce cell migration during inflammation which plays an important role in vascular disease. CCR8 has a similarity score of 0.49 with the known disease gene CX3CR1 based on a single 7tm_{—}1 domain (PF00001). An insulin receptor substrate, IRS2 was predicted in the nearest and adjacent NN mapping approaches. Like the known disease gene IRS1, IRS2 has IRS (PF02147) and PH (PF00169) domains, with a similarity score of 0.74. Under the adjacent NN mapping approach, the genes that have good biological and genetic support were LDL receptors: LRP5L low density lipoprotein receptor-related protein 5-like, LRP11 low density lipoprotein receptor-related protein 11; and LRP12 low density lipoprotein-related protein 12. LDL is an important component in the manifestation of atherosclerosis. At the SNP level, SNP rs9478945 is located in an exon of LRP11, and is a missense mutation changing a threonine to a methionine (C to T, Thr 281 to Met), but has been ascribed as a “natural variant”. These genes have a single domain in common with the known disease gene LRP6, LDL receptor-related protein 6: either the LDL receptor A (PF00057) or LDL receptor B (PF00058) domain. The similarity scores between the LRP6 and these candidates range between 0.57 and 0.43. No functional role has been ascribed to Thr 281 but the mutation could remove a potential phosphorylation site or substitution of the Met could introduce a site of potential oxidative modification. A CMP prediction with weaker genetic support is ABCAl2, ATP-binding cassette 12, a probable transporter involved in lipid homeostasis that has a similarity score of 0.56 with known disease gene ABCA1. SNP rs17493319 is located in the first intron of this gene, with a weak association significance of 7×10-4.

TABLE 9
Top CAD predictions made by CPS and CMP

| | | Mapping | | | | | | |
|---|---|---|---|---|---|---|---|---|
| | | Approach | | | Biological | Genetic | | |
| Group | Method | 1M | Adj | N | Support | Support | Genes | Loci |
| Type II diabetes | CPSab | ✓ | | | ♦ | ▪ | CACNA1D | 3p21.1b |
| mellitus | CPSk | ✓ | ✓ | | ♦♦♦♦ | ▪▪ | CACNA1E | 1q25.3b |
| pathway^{a} | | ✓ | | | ♦ | ▪ | GCK | 7p13d |
| | | ✓ | | | ♦ | ▪ | IKBKB | 8p11.21a |
| | | ✓ | | | ♦ | ▪ | INS | 11p15.5a |
| | | ✓ | | | ♦ | ▪ | IPF1 | 13q12.2b |
| | | ✓ | | | ♦ | ▪ | KCNJ11 | 11p15.1d |
| | | ✓ | | | ♦ | ▪ | ABCC8 | 11p15.1d |
| | | ✓ | | | ♦ | ▪ | TNF | 6p21.33a |
| | | ✓ | ✓ | | ♦♦♦♦ | ▪▪ | IRS2 | 13q34a |
| | | ✓ | | | ♦ | ▪ | ADIPOQ | 3q27.3a |
| | | ✓ | | | ♦ | ▪ | PIK3R5 | 17p13.1c |
| | | ✓ | | | ♦ | ▪ | MAFA | 8q24.3f |
| Insulin signaling | CPSk | | | ✓ | ♦♦♦♦ | ▪ | GRB2 | 17q25.1c |
| pathway^{a} | | ✓ | ✓ | ✓ | ♦♦♦♦ | ▪▪ | PYGB | 20p11.21a |
| | | ✓ | ✓ | ✓ | ♦♦♦♦ | ▪▪ | IRS2 | 13q34a |
| | | ✓ | ✓ | ✓ | ♦♦♦♦ | ▪▪ | SORBS1 | 10q23.33d |
| | | | | ✓ | ♦♦♦♦ | ▪ | KIAA1303 | 17q25.3e |
| | | ✓ | | | ♦♦ | ▪ | EXOC7 | 17q25.1d |
| ADAMTS family | CMPab | | ✓ | ✓ | ▪▪▪▪* | ▪ | ADAMTS7 | 15q25.1a |
| members | | | ✓ | ✓ | ▪▪▪▪* | ▪ | ADAMTS2 | 5q35.3d |
| | | | ✓ | | ▪▪▪▪* | ▪ | ADAMTS18 | 16q23.1c |
| | | | ✓ | ✓ | ▪▪▪ | ▪ | THSD4 | 15q23b |
| Integrins | CMPab | ✓ | | | ▪▪▪▪* | ▪ | ITGB1 | 10p11.22b |
| | | ✓ | | | ▪▪▪▪* | ▪▪ | ITGB2 | 17q21.32a |
| | | ✓ | | | ▪▪▪▪* | ▪ | ITGB3 | 17q21.32a |
| | | ✓ | | | ▪▪▪ | ▪▪ | ITGB4 | 17q25.1c-q25.1d |
| | | ✓ | | | ▪▪▪▪* | ▪▪ | ITGB5 | 3q21.2a |
| Matrix | CMPab | ✓ | | | ▪▪▪▪* | ▪▪ | MMP15 | 16q13d |
| metalloproteases^{b} | | ✓ | | | ▪▪▪▪* | ▪▪ | MMP19 | 12q13.2c |
| Cell-collagen | CMPab | | ✓ | ✓ | ▪ | ▪ | TGFBI | 5q31.1f-q31.2a |
| interaction | | | ✓ | ✓ | ▪ | ▪ | POSTN | 13q13.3c |
| TGFβ signalling | CMPab | | ✓ | | □□□□ | ▪ | SMAD3 | 15q22.33b-q22.33c |
| | | | ✓ | | □□□□ | ▪ | SMAD5 | 5q31.2a |
| Phospholipases | CMPab | ✓ | | | ▪▪▪▪* | ▪ | PLCB3 | 11q13.1b |
| | | ✓ | | | ▪▪▪▪* | ▪ | PLCB2 | 15q15.1a |
| | | | ✓ | | | ▪ | PLCG2 | 16q23.2b-q23.3a |
| | | ✓ | ✓ | | ▪▪▪▪* | ▪ | PLCZ1 | 12p12.3b |
| DAG kinases | CMPab | | ✓ | | ▪▪▪ | ▪ | DGKB | 7p21.2a |
| | | | ✓ | | ▪▪▪ | ▪ | DGKH | 13q14.11c |
| Protein kinase C- | CMPab | | ✓ | | ▪▪▪▪* | ▪ | CDC42BPB | 14q32.32a |
| like | | | ✓ | | ▪▪▪▪* | ▪ | CIT | 12q24.32a |
| Band4.1-like | CMPab | ✓ | ✓ | ✓ | ▪▪▪▪* | ▪ | EPB41 | 1p35.3a |
| | | ✓ | | | ▪▪▪▪* | ▪ | EPB41L1 | 20q11.23a |
| | | ✓ | | | ▪▪▪▪* | ▪ | EPB41L4B | 9q31.3a |
| | | ✓ | ✓ | ✓ | ▪▪▪▪* | ▪ | FARP1 | 13q32.2b |
| | | ✓ | ✓ | | ▪▪ | ▪ | PTPN3 | 9q31.3a |
| | | ✓ | ✓ | | ▪▪ | ▪ | RDX | 11q22.3d |
| FastK-like | CMPab | ✓ | | | ▪▪ | ▪ | FASTK | 7q36.1d |
| | | ✓ | | | ▪▪ | ▪ | TBRG4 | 7p13c |
| Adhesion | CMPab | ✓ | | | ▪▪▪ | ▪▪ | CELSR2 | 1p13.3b |
| GCPRs | | ✓ | | | ▪▪▪ | ▪▪ | BAI1 | 8p24.3e |
| GEFs | CMPab | ✓ | | | □□□ | ▪▪▪ | KALRN | 3q21.1c-q21.2a |
| | | ✓ | | | □□□ | ▪▪▪ | PLEKHG1 | 6q25.1b |
| CUB/sushi | CMPab | ✓ | | | □□□□ | ▪▪▪ | CSMD2 | 1p35.1a-p34.3f |
| adhesion | | ✓ | | | □□□□ | ▪▪▪ | SEZ6L | 22q12.1a |
| cadherins | CMPab | ✓ | | | □□□□ | ▪▪ | CDH4 | 20q13.33b-q13.33c |
| | | ✓ | | | □□□□ | ▪▪ | CDH13 | 16q23.3a-q23.3b |
| | | | | | | | DSC3 | 18q12.1d |
| Calpains | CMPab | ✓ | | | ▪▪▪ | ▪▪ | CAPN9 | 1q42.2a |
| | | ✓ | | | ▪▪▪ | ▪▪ | CAPN11 | 6p21.1b |
| | | ✓ | | | ▪ | ▪▪ | CAPN2 | 1q41e |
| | | | | | | | &/or | |
| | | ✓ | | | ▪ | ▪▪ | CAPN8 | 1q41e |
| Insulin | CMPab | | ✓ | | □□□□ | ▪▪ | IRS1 | 2q36.3b |
| signaling^{a} | | | ✓ | | □□□□ | ▪▪ | IRS2 | 13q34a |
| Acetylcholine | CMPab | ✓ | | | □□□□ | ▪▪ | CHRNA3 | 15q25.1a |
| receptor | | | | | | | &/or | |
| subunits | | ✓ | | | □□□□ | ▪▪ | CHRNA5 | 15q25.1a |
| | | | | | | | &/or | |
| | | ✓ | | | □□□□ | ▪▪ | CHRNB4 | 15q25.1a |
| | | ✓ | | | □□□□ | ▪▪ | CHRNE | 17p13.2b |
| Heat shock | CMPab | ✓ | | | □□□□ | ▪▪ | DNAJA4 | 15q25.1a |
| proteins | | ✓ | | | □□□□ | ▪▪ | DNAJB13 | 11q13.4b |
| Adaptins | CMPab | ✓ | | | ▪▪▪▪ | ▪ | GGA1 | 22q13.1a |
| | | ✓ | | | ▪▪▪▪ | ▪ | GGA3 | 17q25.1c |
| Exosome | CMPab | | ✓ | | ▪ | ▪ | EXOSC8 | 13q13.3b |
| components | | | ✓ | | ▪ | ▪ | EXOSC9 | 4q27 |
| ATP-dependent | CMPab | | ✓ | | ▪ | ▪▪ | CHD1 | 5q21.1a |
| chromatin | | | ✓ | | ▪ | ▪▪ | BTAF1 | 10q23.32b |
| remodelling | | | | | | | | |
| RNA editing | CMPab | | | ✓ | ▪ | ▪ | ADARB1 | 21q22.3e |
| | | | | ✓ | ▪ | ▪ | ADARB2 | 10p15.3c-3b |
| Plasminogen | CMPk | ✓ | | | | ▪ | PLG | 6q26a |
| and LPA | | ✓ | ✓ | | | ▪ | LPAL2 | 6q25.3f |
| Low-density | CMPk | ✓ | ✓ | | | ▪▪▪ | LRP5L | 22q11.23c |
| lipoprotein | | ✓ | ✓ | | ∘ | ▪▪▪ | ITGB5 | 3q21.2a |
| receptors | | | ✓ | | | ▪▪ | LRP12 | 8q22.3d |
| | | ✓ | ✓ | ✓ | ∘ | ▪▪ | CELSR2 | 1p13.3b |
| | | ✓ | | | | ▪ | LDLRAD3 | 11p13a |
| | | ✓ | | | | ▪ | THBD | 20p11.21c |
| | | ✓ | ✓ | ✓ | | ▪ | LRP11 | 6q25.1a |
| Insulin receptor | CMPk | ✓ | ✓ | ✓ | | ▪▪ | IRS2 | 13q34a |
| Matrix | CMPk | ✓ | | | | ▪▪ | MMP15 | 16q13d |
| metalloproteases | | ✓ | | | ∘ | ▪▪ | MMP19 | 12q13.2c |
| ABC transporter | CMPk | ✓ | ✓ | ✓ | | ▪ | ABCA12 | 2q35a |
| GPCR | CMPk | ✓ | | | | ▪▪ | CCR8 | 3p22.1c |

Bolded genes are predicted independently by more than one method. Loci in bold have previously been associated with the disease.
Abbreviations.
Method: CMPab - CMP ab initio, CMPk - CMP known mode, CPSab - CPS ab initio, CPSk - CPS known mode.
Genetic support: HS ▪▪▪▪, MHS - ▪▪▪, MWS - ▪▪, WS - ▪
Key to biological support (the present invention's scores): CMPab: ▪▪▪▪* - log χ² ≧ 9, ▪▪▪▪ - 8 ≦ log χ² < 9, ▪▪▪ - 7 ≦ log χ² < 8, ▪▪ - 6 ≦ log χ² < 7, ▪ - 5 ≦ log χ² < 6.
Lower χ² values considered for more genetically significant data based on statistics (≧ MWS) or proximity: □□□□ - 4 ≦ log χ² < 5, □□□ - 3 ≦ log χ² < 4.
Lower χ² values considered for single domain proteins ▴ - log χ² > 2.
CMPk: - Sc > 0.7, - Sc > 0.6, - Sc > 0.5, - Sc > 0.4, ∘ - Sc > 0.25.
CPS: ♦♦♦♦ - p < 0.05 and Top 5, ♦♦♦ - p < 0.05 and Top 10, ♦♦ - Top 5, ♦ - p < 0.05.
^{a}including known disease gene IRS1
^{b}including known disease gene MMP3

TABLE 10
CAD CMP known results

| | | | | | Nearest | | | | | | Adjacent | | | | | | 1Mbp | | | | | |
|---|---|---|---|---|---|---|---|---|---|---|---|---|---|---|---|---|---|---|---|---|---|---|
| | | Known | | Common | MHS | | MWS | | WS | | MHS | | MWS | | WS | | MHS | | MWS | | WS | |
| Locus | Gene | Gene | Score | Domains | S | C | S | C | S | C | S | C | S | C | S | C | S | C | S | C | S | C |
| 22q11.23c | LRP5L | LRP6 | 0.433 | Ldl_recept_b | 0 | 0 | 0 | 0 | 0 | 0 | 1 | 1 | 1 | 1 | 3 | 2 | 0 | 0 | 0 | 0 | 1 | 1 |
| 3q21.2a | ITGB5 | LRP6 | 0.316 | EGF | 0 | 0 | 0 | 0 | 0 | 0 | 1 | 1 | 1 | 1 | 1 | 1 | 1 | 1 | 1 | 1 | 1 | 1 |
| 13q34a | IRS2 | IRS1 | 0.742 | IRS\|PH | 0 | 0 | 1 | 1 | 1 | 1 | 0 | 0 | 1 | 1 | 1 | 1 | 0 | 0 | 1 | 1 | 1 | 1 |
| 8q22.3d | LRP12 | LRP6 | 0.572 | Ldl_recept_a | 0 | 0 | 0 | 0 | 0 | 0 | 0 | 0 | 2 | 1 | 7 | 1 | 0 | 0 | 0 | 0 | 0 | 0 |
| 1p13.3b | CELSR2 | LRP6 | 0.360 | EGF | 0 | 0 | 1 | 1 | 1 | 1 | 0 | 0 | 2 | 1 | 2 | 1 | 0 | 0 | 2 | 1 | 2 | 1 |
| 3p22.1c | CCR8 | CX3CR1 | 0.487 | 7tm_1 | 0 | 0 | 0 | 0 | 0 | 0 | 0 | 0 | 0 | 0 | 0 | 0 | 0 | 0 | 3 | 1 | 8 | 2 |
| 16q13d | MMP15 | MMP3 | 0.451 | Hemopexin\| | 0 | 0 | 0 | 0 | 0 | 0 | 0 | 0 | 0 | 0 | 0 | 0 | 0 | 0 | 1 | 1 | 1 | 1 |
| | | | | PG_binding_1\| | | | | | | | | | | | | | | | | | | |
| | | | | Peptidase_M10 | | | | | | | | | | | | | | | | | | |
| 12q13.2c | MMP19 | MMP3 | 0.370 | Hemopexin | 0 | 0 | 0 | 0 | 0 | 0 | 0 | 0 | 0 | 0 | 0 | 0 | 0 | 0 | 1 | 1 | 1 | 1 |
| | | | | \|PG_binding_1\| | | | | | | | | | | | | | | | | | | |
| | | | | Peptidase_M10 | | | | | | | | | | | | | | | | | | |
| 6q26a | PLG | LPA | 0.852 | Kringle\|Trypsin | 0 | 0 | 0 | 0 | 0 | 0 | 0 | 0 | 0 | 0 | 0 | 0 | 0 | 0 | 0 | 0 | 2 | 2 |
| 6q25.3f | LPAL2 | LPA | 0.851 | Kringle | 0 | 0 | 0 | 0 | 0 | 0 | 0 | 0 | 0 | 0 | 1 | 1 | 0 | 0 | 0 | 0 | 3 | 3 |
| 11p13a | LDLRAD3 | LRP6 | 0.563 | Ldl_recept_a | 0 | 0 | 0 | 0 | 0 | 0 | 0 | 0 | 0 | 0 | 0 | 0 | 0 | 0 | 0 | 0 | 1 | 1 |
| 2q35a | ABCA12 | ABCA1 | 0.557 | ABC_tran | 0 | 0 | 0 | 0 | 1 | 1 | 0 | 0 | 0 | 0 | 1 | 1 | 0 | 0 | 0 | 0 | 1 | 1 |
| 20p11.21c | THBD | LRP6 | 0.536 | EGF | 0 | 0 | 0 | 0 | 0 | 0 | 0 | 0 | 0 | 0 | 0 | 0 | 0 | 0 | 0 | 0 | 1 | 1 |
| 6q25.1a | LRP11 | LRP6 | 0.450 | Ldl_recept_a | 0 | 0 | 0 | 0 | 1 | 1 | 0 | 0 | 0 | 0 | 1 | 1 | 0 | 0 | 0 | 0 | 1 | 1 |

S - number of SNPs
C - number of clusters formed by SNPs
Genes in bold are those with SNPs within gene boundaries

The predicted genes from CMP ab initio input mode have common themes cell-cell, ECM adhesion and its remodeling featuring prominently as evidenced by integrins, proteins of the actin cytoskeleton, and zinc metalloproteases. Those with the strongest genetic support were guanonucleotide exchange factors and the vascular adhesion factors SEZ6DL and CSMD2. Cell division proteins and phospholipases were also among highly favored candidates on a biological basis. Adhesion between the cell and the extracellular matrix was implicated by multiple integrins and matrix metalloproteases as well as by TGFBI and PSTN. TGFBI binds to type I, II, and IV collagens. This adhesion protein may play an important role in cell-collagen interactions. The matrix metalloproteases were amongst the strongest CMP ab initio results. Interestingly, the original CAD disease gene MMP3 was not predicted. Periostin (PSTN) binds to heparin, inducing cell attachment and spreading and plays a role in cell adhesion. PSTN may play a role in extracellular matrix mineralization. Other adhesion genes were adhesion GPCRs, cadherins and CUB/sushi group. Both are involved in leukocyte adhesion. Involvement of phosopholipids was implicated by multiple phospholipid-binding domains from the C clan and generation by phospholipases. Cytoskeletal organization and cell motility was implicated by the protein kinase C-like genes. CDC42BP may act as a downstream effector of CDC42 in cytoskeletal reorganization, and contributes to the actomyosin contractility required for cell invasion. CIT may play a role in cytokinesis as a putative effector that binds Rho and Rac1. TGF-β signaling was implicated by TGFBI and SMAD3 and SHADS. TGF-f3 signaling has a profound impact on the regulation of the actin cytoskeleton, which supports various physiological and developmental processes such as cell motility, differentiation changes and tissue organization. The regulatory enzymes of the Ras family, namely Rab, Ran and Rho GTPases regulate TGF-f3 signaling during receptor endocytosis, Smad trafficking and cross-talk with the actin cytoskeleton, respectively. Two ab initio predictions have previously been associated with CAD. IRS1 is a known disease gene. A genetic defect of insulin action (the g972R Insulin Receptor Substrate 1 variant) may sustain endothelial dysfunction, the first defect of vascular homeostasis in the road to atherosclerosis. Genetic variations in CHRNA3 have previously been associated with susceptibility to peripheral arterial occlusive disease type 2 (PAOD2, [MIM 612052]), which often coexists with coronary artery disease and cerebrovascular disease. PAOD results from atherosclerosis of large and medium peripheral arteries, as well as the aorta.

At the domain level, the common themes enriched in CMP ab initio input mode were Ca²⁺-binding implicated by C2 and EF hands domains, and phospholipid binding implicated by C1 and C2 domains. The C2 domain is a Ca²⁺-dependent membrane-targeting module found in many cellular proteins involved in signal transduction or membrane trafficking. C2 domains are unique among membrane targeting domains in that they show wide range of lipid selectivity for the major components of cell membranes, including phosphatidylserine and phosphatidylcholine. C1_{—}1 domains bind diacylglycerol (DAG), an important second messenger. Phorbol esters (PE) are analogues of DAG and potent tumour promoters that cause a variety of physiological changes when administered to both cells and tissues. DAG activates a family of serine/threonine protein kinases, collectively known as protein kinase C (PKC).

### Crohn's Disease (CD)

For the CD phenotype, CPS predicted up to 65 genes using known disease genes input mode; and up to 162 genes in ab initio input mode (Table 5). For CMP using known disease genes input mode up to 6 genes were predicted. CMP in ab initio input mode, the number of predictions was at most 1807, with up to 66 genes reaching the arbitrary threshold χ2 max_unique (Table 7).

Of the known five known disease genes used as seeds from OMIM, up to three IL23R DLG5, and CARD15 were in gene search spaces mapped by the present inventors. CMP ab initio input mode predicted DLG5 and CARD15, but the results do not pass the threshold x2 max_unique. IL23R was predicted in both CPS known disease genes input mode and CPS ab initio input mode in the “Cytokine-cytokine receptor interaction” pathway and the “Jak-STAT signaling pathway”, but were not significant.

A highly significant region implicated in the WTCCC study for the CD phenotype was in gene ATG16L1 (p=7.1×10⁻¹⁴). A second region (p=2.7×10-7) was intergenic to ZNF365 and ATQL4. Four other significantly associated regions include SNPs around IRGM (p=5.1×10-8), in BSN (p=7.7×10-7) but near MST/, a region near NKX2-3 (p=1.4×10-8) and one near PTPN2 (p=4.6×10-8). Regions of more modest associations were mapped to the HLA-locus (p=8.7×10-7), TNFAIP3 (p=4.42×10-6), within TNFSF 15 (p=9.0×10-5), within STAT3 (p=3.1×10-5), and near PTPN11 (p=1.5×10-3). Of these 12 candidates, 9 were annotated within the database of the present invention with either a domain or a pathway. CPS in known disease gene input mode predicted STAT3 as it shares common pathways “Role of ERBB2 in Signal Transduction and Oncology”, “IL 6 signaling pathway” and “Jak-STAT signaling pathway” with known disease gene IL6 and “Jak-STAT signaling pathway” with IL23R. In CMP ab initio input mode, STAT3 was also predicted along with other STAT proteins, but the genes MST/, PTPN2 and TNFAIP3 do not reach the χ2 max_unique threshold.

In known disease gene input mode, the top ranking and significant pathways in CPS using the nearest mapping were the “Cytokine-cytokine receptor interaction” and “Jak-STAT signaling pathway”. The genes implicated by these two pathways were IL12RB2, an interleukin 12 receptor subunit and IL12B, an interleukin 12 subunit. TNFSF18, a cytokine belonging to the tumor necrosis factor (TNF) ligand family. The adjacent mapping had similar results, with the inclusion of the prediction of OSMR, a subunit of the IL31 receptor that binds to STAT3. The BY mapping approaches decreased the significance of these top ranking pathways; instead the predictions of the 1 Mbp BY mapping were hematopoeitic. CSF2 and CSF3, EPO, IL3/4/5/8 and CCL3 were predicted.

CPS in ab initio input mode predicted pathways at the higher significance levels (HS and MHS) similar to those predicted by CPS in known disease gene input mode, as the IL23R gene were in the search space. However, at the MWS and WS levels different pathways were predicted. A top ranking pathway that is significant in the WS set was the “Neuroactive ligand17 receptor interaction” in the nearest and adjacent mapping approaches. Increasing to the 1 Mbp BY mapping, the pathway was no longer significant. Instead, pathways related to amino acid and lipid metabolism appear, such as “Phenylalanine, tyrosine and tryptophan biosynthesis”, “Eicosanoid Metabolism” and “Alanine and aspartate metabolism”.

CMP using known disease gene input mode as seeds had very few predictions, all with known disease gene DLG5. The highest score and the one with the most genetic support was with RAPGEF6 (0.336), sharing a PDZ (PF00595) domain.

The CMP ab initio input mode predicted the strongest genetic support were glutathione peroxidases GPX1 and GPX3. These genes were ranked number one by CMP ab initio input mode among single domain proteins. The glutathione peroxidases conjugate peroxide with glutathione to maintain cellular redox homeostasis93. GPX1 performs this role in the cytoplasm, and GPX3 in plasma. Upregulation of the homologous mitochrondrial gene GPX2 has been demonstrated in a mouse model and in colonic tissue of human patients. For multidomain proteins, CMP ab initio input mode made a total of 66 predictions above the arbitrary threshold. A total of 8 gene clusters were predicted when SNPs were mapped to the nearest gene, 11 gene clusters when the four adjacent genes were considered, and 16 gene clusters when about 1 Mbp intervals were considered.

Several themes were apparent in the CMP ab initio input mode results for the CD phenotype including: tissue homeostasis through WNT signaling, dynamics of the actin cytoskeleton, neuronal regulation of gut motility, wound healing, and possibly vesicular transport. Cell renewal in the intestinal epithelium is controlled by Ephrin and WNT signaling. WNT family members are secreted glycoproteins which orchestrate embryogenesis, and tissue homeostasis. WNT signaling cascades network with Notch, FGF, BMP and Hedgehog signaling cascades to regulate the balance of stem cells and progenitor cells. Candidates in these pathways include the WNT family members FZD1 and FZD2, NOTCH1 and NOTCH2, as well as BMP2 and BMP4. Defects in wound healing have also been linked to CD and this is supported by multiple candidates including ephrin receptors, transglutaminases, the Von Willebrand factor group, and laminins. For example, Ephrin-B2 is differentially expressed in the intestinal epithelium in Crohn's disease and contributes to accelerated epithelial wound healing in vitro. Ephrin receptors are specifically involved reorganization of the actin cytoskeleton. Other genes likely involved in actin cytoskeletal reorganization are four Kelch-like proteins, two Ras-like GTPases: R-Ras96 and CDC42, as well as two CDC42-binding proteins, and two anthrax toxin receptors. Of the many implicated Ras-like GTPases, RhoA is involved in Ephrin forward signalling and RheB is involved in signalling by the insulin receptor INSR, which is also a predicted candidate. There are eight Rab GTPases which are implicated in vesicle trafficking: a process also implicated by the vesicle-fusing ATPases, NSF and LOC7298806. RhoH inhibits RACJ, RHOA and CDC42. Oxidative modifications to cytoskeletal proteins have also been observed in the superphenotype irritable bowel disorder (IBD, [MIM 266600]), which also includes ulcerative colitis. Another candidate, tubulin, was shown to be carbonylated.

Neuronal regulation of gut motility is implicated via the inhibitory metabotropic glutamate receptors (mGluR groups II and III) and the β subunits of GABAA receptors. In addition, one of the Kelch-like proteins (KLHL24) interacts with the inotropic glutamate receptor GRIK2, which may also be related to this theme. Eight genes encode mGluR in the human genome. Of these, three genes belonging to group I are excitatory. Of the five inhibitory mGluR genes, four are significant for the CD phenotype when SNPs are mapped to adjacent genes. Group II and group III mGluRs are linked to the inhibition of the cyclic AMP cascade, but differ in their agonist selectivities. Elevated cAMP levels have recently been linked to Crohn's disease in a mouse model and cAMP signalling was also shown to be associated with dysregulation of purine gene expression in Crohn's disease but not in Ulcerative colitis. Other predicted candidates which have homologs previously associated with Crohn's disease are the ubiquitin genes UBE1L1 and UBE1L2 and the cadherin genes CHD8 and CDH10. 1 Polymorphisms in E-cadherin (CDH1) have been implicated in increase gut permeability in some patients with Crohn's disease. Autoantibodies against ubiquitination factor E4A (UBE4A) are associated with severity of Crohn's disease. Table 11 detailed the additional genes predicted.

TABLE 11
Top CD predictions made by CPS and CMP

| | | Mapping | | | | | | |
|---|---|---|---|---|---|---|---|---|
| | | Approach | | | Biological | Genetic | | |
| Group | Method | 1M | Adj | N | Support | support | Genes | Loci |
| Jak-STAT | CPSk | ✓ | ✓ | ✓ | ♦♦♦♦ | ▪▪▪▪ | IL12RB2 | 1p31.3a |
| signaling | | ✓ | ✓ | ✓ | ♦♦♦♦ | ▪▪ | IL12B | 5q33.3c |
| pathway^{a,b} | | ✓ | ✓ | ✓ | ♦♦♦♦ | ▪▪ | STAT3 | 17q21.2b |
| | | ✓ | ✓ | ✓ | ♦♦ | ▪ | CSF2 | 5q31.1b |
| | | ✓ | ✓ | ✓ | ♦♦ | ▪ | GRB2 | 17q25.1c |
| | | ✓ | ✓ | ✓ | ♦♦ | ▪ | IFNGR1 | 6q23.3c |
| | | ✓ | ✓ | ✓ | ♦♦ | ▪ | SPRED2 | 2p14c |
| | | ✓ | ✓ | | ♦♦♦♦ | ▪▪▪▪ | OSMR | 5p13.1c |
| Cytokine-cytokine | CPSk | ✓ | ✓ | ✓ | ♦♦♦♦ | ▪▪▪▪ | IL12RB2 | 1p31.3a |
| receptor | | ✓ | ✓ | ✓ | ♦♦♦♦ | ▪▪▪ | TNFSF18 | 1q25.1a |
| interaction^{a,c} | | ✓ | ✓ | ✓ | ♦♦♦♦ | ▪▪ | CCL18 | 17q12b |
| | | ✓ | ✓ | ✓ | ♦♦♦♦ | ▪▪ | IL12B | 5q33.3c |
| | | ✓ | ✓ | ✓ | ♦♦♦♦ | ▪ | BMP2 | 20p12.3b |
| | | ✓ | ✓ | ✓ | ♦♦♦♦ | ▪ | CSF2 | 5q31.1b |
| | | ✓ | ✓ | ✓ | ♦♦♦♦ | ▪ | IFNGR1 | 6q23.3c |
| | | ✓ | ✓ | ✓ | ♦♦♦♦ | ▪ | IL8 | 4q13.3d |
| | | ✓ | ✓ | ✓ | ♦♦♦♦ | ▪ | KDR | 4q12c |
| | | ✓ | ✓ | ✓ | ♦♦♦♦ | ▪ | TNFRSF6B | 20q13.33e |
| | | ✓ | ✓ | ✓ | ♦♦♦♦ | ▪ | IL18RAP | 2q12.1a |
| | | ✓ | ✓ | | ♦♦♦♦ | ▪▪▪▪ | OSMR | 5p13.1c |
| PDZ domain | CMPk | ✓ | | | ∘ | ▪▪ | RAPGEF6 | 5q31.1a |
| contain guanine | | | | | | | | |
| nucleotide | | | | | | | | |
| exchange factor | | | | | | | | |
| Glutathione | CMPab | ✓ | | | ▴ | ▪▪▪ | GPX1 | 3p21.3 |
| peroxidase | | ✓ | | | ▴ | ▪▪▪ | GPX3 | 5q23 |
| inhibitory | CMPab | ✓ | ✓ | | ▪▪▪▪ | ▪▪ | GRM4-III | 6q21.31f-p21.31e |
| metabotropic | | ✓ | ✓ | ✓ | ▪▪▪▪ | ▪▪ | GRM8-III | 7q31.33c |
| glutamate | | ✓ | ✓ | ✓ | ▪▪▪▪ | ▪ | GRM7-III | 3p26.1b-p26.1a |
| receptors | | ✓ | ✓ | | ▪▪▪▪ | ▪ | GRM3-II | 7q21.11g-q21.12a |
| GABA receptor β | CMPab | | ✓ | | ▪▪▪ | ▪ | GABRB1 | 4p12b |
| subunit | | | ✓ | | ▪▪▪ | ▪ | GABRB2 | 5q34a |
| Notch genes | CMPab | ✓ | | | ▪▪▪▪* | ▪ | NOTCH1 | 9q34.3d |
| | | ✓ | | | ▪▪▪▪* | ▪ | NOTCH2 | 1p12a |
| Frizzled genes | CMPab | ✓ | | | □□□□ | ▪▪ | FZD1 | 7q21.13c |
| | | ✓ | | | □□□□ | ▪▪ | FZD8 | 10p11.21b |
| BMP genes | CMPab | | | ✓ | □□□□ | ▪ | BMP2 | 20p12.3b |
| | | | | ✓ | □□□□ | ▪ | BMP4 | 14q22.2b |
| Phospholipases | CMPab | ✓ | ✓ | | ▪▪▪▪* | ▪ | PLCB1 | 20p12.3a |
| | | ✓ | | | ▪▪▪▪* | ▪ | PLCB3 | 11q13.1b |
| | | ✓ | | | ▪▪▪▪* | ▪ | PLCB4 | 20p12.2b |
| | | ✓ | | | ▪▪▪▪* | ▪ | PLCD3 | 17q21.31d |
| | | ✓ | ✓ | | ▪▪▪▪* | ▪ | PLCZ1 | 12p12.3b |
| Autoimmune | CMPab | | ✓ | | ▪▪▪ | ▪ | AIRE1 | 21q22.3d |
| regulation | | | ✓ | | ▪▪▪ | ▪ | SP140 &/or | 2q37.1a |
| | | | ✓ | | ▪▪▪ | ▪ | SP110 | 2q37.1a |
| STATs | CMPab | ✓ | ✓ | | ▪▪▪▪* | ▪▪ | STAT4 | 2q32.3a |
| | | ✓ | ✓ | | ▪▪▪▪* | ▪▪ | STAT3 &/or | 17q21.2b |
| | | ✓ | ✓ | | ▪▪▪▪* | ▪▪ | STAT5A | |
| | | | | | | | &/or | |
| | | ✓ | | | ▪▪▪▪* | ▪▪ | STAT5B | |
| Pkinase_C | CMPab | ✓ | | | ▪▪▪▪* | ▪ | CDC42PBA | 1q42.13a |
| | | ✓ | | | ▪▪▪▪* | ▪ | CDC42PBG | 11q13.1b |
| | | | | | | ▪ | PRKCD | 3p21.1c |
| Tyrosine kinase | CMPab | | ✓ | | ▪▪▪ | ▪ | ERBB4 | 2q34c-q34e |
| receptors | | | ✓ | | ▪▪▪ | ▪ | IGF1R | 15q26.3a-q26.3b |
| | | | ✓ | | ▪▪▪ | ▪ | INSR | 19p13.2e |
| Ephrin receptors | CMPab | | ✓ | | ▪▪▪▪ | ▪ | EPHA5 | 4q13.1f |
| (Tyr kinase) | | | ✓ | | ▪▪▪▪ | ▪ | EPHB4 | 7q22.1c |
| Band 4.1 | CMPab | | ✓ | | ▪▪▪ | ▪ | EPB41L4B | 9q31.3a |
| cytoskeletal | | | ✓ | | ▪▪▪ | ▪ | FRMD4A | 10p13d-p13c |
| proteins | | | ✓ | | ▪▪▪ | ▪ | RDX | 11q22.3d |
| Reorganization of | CMPab | ✓ | | | ▪▪▪▪ | ▪ | ANTXR1 | 2p14a |
| actin cytoskeleton | | ✓ | | | ▪▪▪▪ | ▪ | ANTXR2 | 4q21.21b |
| Actin cytoskeleton | CMPab | | ✓ | | ▪▪ | ▪ | KLHL1 | 13q21.33b |
| Kelch proteins | | | ✓ | | ▪▪ | ▪ | KLHL2 | 4q32.3b |
| | | | ✓ | | ▪▪ | ▪ | KLHL20 | 1q25.1a |
| | | | ✓ | | ▪▪ | ▪ | KLHL24 | 3q27.1a |
| Glucose | CMPab | ✓ | | | ▪▪▪▪* | ▪ | PGM1 | 1p31.3c |
| metabolism | | ✓ | | | ▪▪▪▪* | ▪ | PGM5 | 9q13a-q13b |
| laminins | CMPab | | ✓ | | ▪▪▪▪* | ▪ | LAMA1 | 18p11.31a |
| | | | | | ▪▪▪▪* | ▪ | LAMA3 | 18q11.2b-q11.2c |
| transglutaminases | CMPab | ✓ | | | ▪▪▪▪* | ▪ | TGM1 | 14q12a |
| | | ✓ | | | ▪▪▪▪* | ▪ | TGM4 | 3p21.31k |
| | | ✓ | | | ▪▪▪▪* | ▪ | TGM3 &/or | 20p13d |
| | | ✓ | | | ▪▪▪▪* | ▪ | TGM6 | |
| Von Willebrand like | CMPab | | ✓ | | ▪▪▪ | ▪ | VWF | 12p13.31e |
| | | | ✓ | | ▪▪▪ | ▪ | ZAN | 7q22.1c |
| Vesicle-fusing | CMPab | ✓ | | | ▪▪▪▪* | ▪ | NSF | 17q21.32a |
| ATPases | | ✓ | | | ▪▪▪▪* | ▪ | LOC728806 | 17q21.31e-q21.32a |
| Synthesis of N- | CMPab | ✓ | | | ▪▪▪▪* | ▪ | MAN2A1 | 5q21.3e |
| glycans | | ✓ | | | ▪▪▪▪* | ▪ | MAN2A2 | 15q26.1c |
| tubulins | CMPab | ✓ | | | □□□□ | ▪▪ | TUBB2A | 6p25.2b |
| | | | | | | | &/or | |
| | | ✓ | | | □□□□ | ▪▪ | TUBB2B | |
| | | ✓ | | | □□□□ | ▪▪ | TUBG2 &/or | 17q21.21a |
| | | ✓ | | | □□□□ | ▪▪ | TUBG1 | |
| | | ✓ | | | □□□□ | ▪▪ | TUBB6 | 18p11.21e |
| TPR repeat- | CMPab | | ✓ | ✓ | ▪▪▪ | ▪ | TMTC1 | 12p11.22a |
| containing | | | ✓ | ✓ | ▪▪▪ | ▪ | TMTC2 | 12q21.31c |
| | | | | ✓ | □□□ | ▪ | TTC14 | 3q26.33b |
| Ubiquitin | CMPab | ✓ | | | ▪▪▪▪ | ▪ | UBA7 | 3p21.31c |
| | | ✓ | | | ▪▪▪▪ | ▪ | UBA6 | 4q13.2b |
| semaphorins | CMPab | | | ✓ | □□□□ | ▪ | SEMA4F | 2p13.1a |
| | | | | ✓ | □□□□ | ▪ | SEMA5A | 5p15.2d |
| cadherins | CMPab | | | ✓ | □□□□ | ▪ | CDH8 | 16q21c |
| | | | | ✓ | □□□□ | ▪ | CDH10 | 5p14.2a |
| ETS transcription | CMPab | ✓ | | | □□□□ | ▪▪ | ERG &/or | 21q22.2a |
| factors | | ✓ | | | □□□□ | ▪▪ | ETS2 | 21q22.2a |
| | | ✓ | ✓ | | □□□□ | ▪▪ | ETV7 | 6p21.31a |
| | | ✓ | ✓ | | □□□□ | ▪▪ | GABPA | 21q21.3a |
| Transcriptional | CMPab | ✓ | | | □□□□ | ▪▪ | MIER1 | 1p31.3a |
| repression | | ✓ | | | □□□□ | ▪▪ | MIER2 | 19p13.3j |
| Zn finger | CMPab | | | ✓ | □□□ | ▪▪ | ZNF33A | 10p11.21a |
| transcription | | | | ✓ | □□□ | ▪▪ | ZNF221 | 19q13.31b |
| factors | | | | ✓ | □□□ | ▪▪ | ZNF300 | 5q33.1d |
| Ras-like GTPases | CMPab | ✓ | ✓ | | ▴ | ▪ | RHOA | 3p21.3d |
| | | ✓ | | | ▴ | ▪ | RHEB | 7q36.1d |
| | | ✓ | | | ▴ | ▪ | RRAS* | 19q13.33b |

Bolded genes are predicted independently by more than one method. Loci in bold have previously been associated with the disease.
Abbreviations.
Method: CMPab - CMP ab initio, CMPk - CMP known mode, CPSab - CPS ab initio, CPSk - CPS known mode.
Genetic support: HS ▪▪▪▪, MHS - ▪▪▪, MWS - ▪▪, WS - ▪.
Key to biological support (the present invention's scores): CMPab: ▪▪▪▪* - log χ2 ≧ 9, ▪▪▪▪- 8 ≦ log χ2 < 9, ▪▪▪- 7 ≦ log χ2 < 8, ▪▪- 6 ≦ log χ2 < 7, ▪- 5 ≦ log χ2 < 6.
Lower χ2 values considered for more genetically significant data based on statistics (≧ MWS) or proximity: □□□□- 4 ≦ log χ2 < 5, □□□- 3 ≦ log χ2 < 4. Lower χ2 values considered for single domain proteins ▴- log χ2 > 2.
CMPk: - Sc > 0.7, - Sc > 0.6, - Sc > 0.5, - Sc > 0.4, ∘- Sc > 0.25. CPS: ♦♦♦♦- p < 0.05 and Top 5, ♦♦♦- p < 0.05 and Top 10, ♦♦- Top 5, ♦- p < 0.05.
^{a}Includes known disease gene IL23R.
^{b}CNTF CSF3 EPO EPOR IL2 IL3 IL4 IL5 IL13 MYC PIM1 PIK3R1 PRL STAT4 STAT5A STAT5B PIK3R3 PIAS1 SOCS3 SPRY2 STAM2 ISGF3G IL20RA IL21 IL22RA2.
^{c}Full list: DIRAS2, RAB6B, RAB3C, LOC643752, RAB5C, RAB3D, RALB, RAB1A, RAB8B, RHOH, CDC42, RIT2, RAN, RBJ, RAB4A, RAB20

### Hypertension (HT)

CPS predicted up to 48 genes using known disease gene input mode and up to 77 genes in ab initio input mode. Up to 23 common pathways reaching significance using the 0.1 Mbp BY SNP mapping approach (Table 7). Using known disease genes input mode, CMP predicted up to 70 genes depending on the statistical significance of the SNP set and the mapping approach used. CMP ab initio input mode predictions considered at most about 1337 genes, with about 73 over an arbitrary χ2 max_unique threshold (Table 7). The most significant predictions are shown in Table 12.

The 23 hypertension-implicated genes listed in OMIM were involved in the calcium signaling pathway, renin-angiotensin system and hormone metabolism. These pathways regulate blood pressure and blood volume. Of these known disease genes, four genes were in the search spaces: AGT, AGTR1, EPHX1, and PTGIS. AGT and AGTR1 are part of many common pathways and were subsequently predicted by CPS in known disease gene input mode. PTGIS and EPHX1 also share a common pathway so are both predicted by CPS known. In ab initio input mode, AGT and AGTR1 were predicted by numerous significant angiotensin related pathways. PTGIS and EPHX1 are predicted by CPS ab initio input mode but the pathways are not statistically significant. None of the genes reached significance in the CMP ab initio input mode, even though they share some common domains with other genes in the search space.

In the WTCCC study, no SNPs reached a significance level p<5×10⁻⁷ (HS) for the hypertension phenotype, but the number of more modest associations were comparable to the other diseases. A potential region of interest with a modest association was on chromosome 1q43 (p=7.7×10⁻⁷) closest to three genes: a cardiac ryanodine receptor, RYR2, a muscarinic cholinergic receptor, CHRM3; and a zona pellucida glycoprotein ZP4. Of these, CPS known disease gene input mode predicted CHRM3 in the pathways “Calcium signaling pathway” (p=0.42) and “Neuroactive ligand-receptor interaction” (p=0.85) using the known disease gene AGTR1, angiotensin receptor 1 as a seed.

The top ranking pathway implicated through CPS using known disease genes as seeds for the MWS set was the “Calcium signaling pathway” using the nearest mapping approach, but was not a statistically significant result (p=1). Calcium signaling and oxidant stress play a major role in vascular biology; inactivation of the sarcoplasmic reticulum Ca2+ pump by reactive oxygen species disables the arteries from contractile activity. Adenylate cyclase ADCY8 was the only gene in the MWS search space implicated by this pathway. However, in the larger WS set, more genes share this pathway including another adenylate cyclase, ADCY4, and two receptors: one that activates adenylate cyclase DRD1, and one that is adenylate cyclase coupled, HTR7. The dopamine D1 receptor DRD1 has been associated with essential hypertension. Adenylyl cyclase is the predominant effector enzyme for G-coupled receptors coupled to the Gs protein. The amount of adenyl cyclase is limiting to the signalling pathway so overexpressing the cardiac isoform causes an increase in cyclic AMP (cAMP) output that is proportional to the level of AC expression. The cholinergic receptor, CHRM3, also in the Ca²⁺ signaling pathway, functions in smooth muscle contraction and vasodilation. The receptor mediates an increase in cellular calcium, and in vascular endothelial cells causes increased synthesis of nitric oxide, which relaxes nearby smooth muscle cells. Under high blood pressure, the expression of the receptor is upregulated. Also predicted and part of this pathway are both ionotropic and metabotropic glutamate receptors (mGluR), implicating the neurotransmitter glu-1-tamate. The mGluR participate in cardiovascular responses through their control of cAMP generation, and group I mGluR play an important role in arterial pressure in rats. Both cAMP and cyclic GMP (cGMP) are involved in vascular smooth muscle relaxation.

The adjacent mapping for the MWS set predicted CDH4, CNTNAP2, and CD276 in the “Cell adhesion molecules (CAMs)” (p=0.04) pathway with the known disease gene SELE. The CDH4 cadherin is thought to play a role in kidney and muscle development. The role of cell-cell adhesion in the vascular phenotype, such as the flexibility and contractility of vascular smooth muscle, has been addressed in studies. Using the WS set, the top ranking pathway implicated was the “Neuroactive ligand-receptor interaction” for the NN and BY mapping approaches, but was only statistically significant in the NN approaches. Many of the genes in this pathway are in those in the “Calcium signaling pathway”. The most significant pathway for the WS set, but was not top ranking, was the “Angiotensin-converting enzyme 2 regulates heart function”, with the CMA1 gene. This chymotryptic serine protease was believed to be responsible for converting angiotensin Ito the vasoactive form in the heart and blood vessels and was implicated in blood pressure control, but other reports claim otherwise and it is true effects remain contentious. In ab initio input mode, CPS predicted similar results. One notable significant and top ranking pathway was the “Gap junction” pathway which contains the mGluRs, guanylate cyclases, adenylate cyclases, and protein kinases.

The CMP using known disease gene input mode predicted was not as concordant with the other methods and did not have particularly high scores. The highest scoring prediction was for RGS8 (0.67), a regulator of G-protein signaling, similar to the known disease gene RGS5. CMP predictions in known disease gene mode are genes containing EGF (PF00008) or WD40 (PF00400) domains.

Control of vascular tone was a theme of the CMP ab initio predictions for hypertension. ADAM metalloproteases, metabotropic glutamate receptors and integrins feature prominently. As in the CPS results, the mGluR and iGluR are predicted. The G6 protein coupled receptor (GPRC6A) is activated by both calcium and amino acids, suggesting it may play a regulatory role in the urea cycle as it is highly expressed in the kidneys. Synaptojanins are inositol 5-phosphatases which have a role in clathrin mediated endocytosis. Foxa transcription factors bind to promoters and enhancers to enable chromatin access for other tissue-specific transcription factors. At the transcriptional level, ASCC1 enhances oxidative stress transcription factors NF-kappa-B, SRF and AP1 transactivation. The exosome complex is widely conserved, functionally versatile, and essential constituent of the machinery regulating gene expression in the nucleus as well as in the cytoplasm. While the most fundamental enzymatic property of exosome is ribonucleolytic activity, its in vivo functions are varied, highly specific, and tightly regulated, and include RNA degradation, processing, and quality control. Recent reports reveal that the exosome also has a prominent role in gene silencing as well as in regulating the expression of a wide variety of noncoding RNAs. Taken together with the emerging notion of pervasive genomewide transcription, these findings indicate that ‘policing the transcriptome’ may well turn out to be the major role of exosome in eukaryotes.

The Helicase_C (PF00271) domain couples an ATPase activity to RNA binding and unwinding. Guanylate_cyc (PF00211) generates second messengers cGMP and cAMP from G-coupled receptor stimulation, that are implicated. Vascular smooth muscle cell (VSMC) contraction and relaxation is regulated by hormonal and neural inputs and initiated by a fall and rise of cytosolic calcium concentration ([Ca2+]) respectively. EGF domains are supported by both the known and ab initio CMP predictions, albeit in different genes, namely integrins and scavenger receptors. The ANF_receptor domain is a generic ligand binding domain. Domains of this fold bind many ligands, several of them amino acids. In this case, both families of receptor bind glutamate.

TABLE 12
Top HT predictions made by CPS and CMP

| | | Mapping | | | | | | |
|---|---|---|---|---|---|---|---|---|
| | | Approach | | | Biological | Genetic | | |
| Group | Method | 1M | Adj | N | Support | Support | Genes | Loci |
| Calcium- | CPSk | ✓ | ✓ | ✓ | ♦♦♦♦ | ▪▪ | ADCY8 | 8q24.2b |
| signalling | CPSab | | | | ♦♦♦♦ | ▪ | ADCY4 | 14q12 |
| pathway | | ✓ | ✓ | ✓ | ♦♦♦♦ | ▪ | DRD1 | 5q35.2c |
| | | ✓ | ✓ | ✓ | ♦♦♦♦ | ▪ | GRIN2A | 16p13.2a |
| | | ✓ | ✓ | ✓ | ♦♦♦♦ | ▪ | GRM5 | 11q14.2b-q14.3a |
| | | ✓ | ✓ | ✓ | ♦♦♦♦ | ▪ | HTR7 | 10q23.31d |
| | | ✓ | ✓ | ✓ | ♦♦♦♦ | ▪ | PPP3CA | 4q23c |
| | | ✓ | ✓ | ✓ | ♦♦♦♦ | ▪ | SLC8A1 | 2p22.1b |
| | | ✓ | ✓ | ✓ | ♦♦♦♦ | ▪ | PLCE1 | 10q23.33b |
| Cell adhesion | CPSk | ✓ | ✓ | | ♦♦♦♦ | ▪▪ | CDH4 | 20q13.3 |
| molecules | | ✓ | ✓ | ✓ | ♦♦♦♦ | ▪▪ | CNTNAP2 | 7q35-q36 |
| (CAMs) | | ✓ | ✓ | | ♦♦♦♦ | ▪▪ | CD276 | 15q23-q24 |
| | | ✓ | | | ♦♦ | ▪▪ | NEO1 | 15q22.3-q23 |
| Angiotensin- | CPSk | ✓ | | | ♦ | ▪▪ | CMA1 | 14q11.2 |
| converting | | | | | | | | |
| enzyme 2 | | | | | | | | |
| regulates heart | | | | | | | | |
| function^{a} | | | | | | | | |
| Neuroactive- | CPSk | ✓ | ✓ | ✓ | ♦♦♦♦ | ▪ | DRD1 | 5q35.2c |
| ligand receptor | CPSab | ✓ | ✓ | ✓ | ♦♦♦♦ | ▪▪ | FSHB | 11p14.1a |
| pathway^{b} | | ✓ | ✓ | ✓ | ♦♦♦♦ | ▪ | GABRA5 | 15q12b |
| | | ✓ | ✓ | ✓ | ♦♦♦♦ | ▪ | HTR7 &/or | 10q23.31d |
| | | ✓ | ✓ | ✓ | ♦♦♦♦ | ▪ | GRID1 | 10q23.1d-q23.2a |
| | | ✓ | ✓ | ✓ | ♦♦♦♦ | ▪ | GRID2 | 4q22.1g-q22.2b |
| | | ✓ | ✓ | ✓ | ♦♦♦♦ | ▪ | GRIN2A | 16p13.2a |
| | | ✓ | ✓ | ✓ | ♦♦♦♦ | ▪▪ | GRM3 | 7q21.11g-q21.12a |
| | | ✓ | ✓ | ✓ | ♦♦♦♦ | ▪ | GRM5 | 11q14.2b-q14.3a |
| | | ✓ | ✓ | ✓ | ♦♦♦♦ | ▪ | GRM8 | 7q31.33c |
| | | ✓ | ✓ | | ♦♦♦♦ | ▪▪ | GRM7 | 3p26.1b-p26.1a |
| | | ✓ | ✓ | ✓ | ♦♦♦♦ | ▪ | LEP | 7q23.1a |
| | | ✓ | ✓ | ✓ | ♦♦♦♦ | ▪ | THRB | 3p24.2b |
| | | ✓ | | | ♦♦ | ▪▪ | CHRM3 | 1q43c |
| | | ✓ | | | ♦♦ | ▪▪ | AGTR1 | 3q24f |
| Gap junction^{c} | CPSab | ✓ | ✓ | ✓ | ♦♦♦♦ | ▪ | DRD1 | 5q35.1 |
| | | ✓ | ✓ | ✓ | ♦♦♦♦ | ▪ | GUCY1A3 | 4q31.1-q31.2 |
| | | ✓ | ✓ | ✓ | ♦♦♦♦ | ▪▪ | ADCY4 | 14q12 |
| | | ✓ | ✓ | ✓ | ♦♦♦♦ | ▪▪ | ADCY8 | 8q24.2b |
| | | ✓ | ✓ | ✓ | ♦♦♦♦ | ▪ | GRM5 | 11q14.2b-q14.3a |
| | | ✓ | ✓ | | ♦♦♦♦ | ▪ | CDC2 | 10q21.1 |
| | | ✓ | ✓ | | ♦♦♦♦ | ▪ | PRKACG | 9q13 |
| | | ✓ | ✓ | | ♦♦♦♦ | ▪ | PRKG1 | 10q11.2 |
| | | ✓ | ✓ | | ♦♦♦♦ | ▪ | MAPK3 | 16p11.2 |
| | | ✓ | ✓ | | ♦♦♦♦ | ▪ | TJP1 | 15q13 |
| regulator of G | CMPk | ✓ | | | | ▪ | RGS8 | 1q25.3c |
| protein signaling | | ✓ | | | | ▪ | RGS3 | 9q32c |
| Dynein | CMPab | ✓ | | | ▪▪▪▪* | ▪ | DNAH8 | 6p21.2b |
| | | ✓ | | | ▪▪▪▪* | ▪ | DNAH2 | 17p13.1d |
| ADAMTS family | CMPab | ✓ | ✓ | ✓ | ▪▪▪▪* | ▪ | ADAMTS1 | 21q21.3a |
| members | | | | | | | &/or | |
| | | ✓ | ✓ | ✓ | ▪▪▪▪* | ▪ | ADAMTS5 | 21q21.3a |
| | | ✓ | ✓ | | ▪▪▪▪* | ▪ | ADAMTS6 | 5q12.3a-q12.3b |
| | | ✓ | ✓ | | ▪▪▪▪* | ▪ | ADAMTS18 | 16q23.1c |
| | | ✓ | | | ▪▪▪▪* | ▪ | ADAMTS15 | 11q24.3c |
| | | ✓ | | | ▪▪▪▪* | ▪ | ADAMTS8 | 3p14.1d |
| | | | | | | | &/or | |
| | | ✓ | | | ▪▪▪▪* | ▪ | ADAMTS9 | 3p14.1d |
| Metabotropic Glu | CMPab | ✓ | ✓ | ✓ | ▪▪▪▪ | ▪▪ | GRM3 | 7q21.11g-q21.12a |
| receptors | | ✓ | ✓ | ✓ | ▪▪▪ | ▪ | GRM5 | 11q14.2b-q14.3a |
| | | ✓ | ✓ | ✓ | ▪▪▪▪ | ▪ | GRM8 | 7q31.33c |
| | | ✓ | ✓ | | ▪▪▪ | ▪▪ | GRM7 | 3p26.1b-p26.1a |
| | | | ✓ | | ▪▪▪ | ▪ | GPRC6A | 6q22.2a |
| δ-subunits of | CMPab | | | ✓ | □□□□ | ▪ | GRID1 | 10q23.1d-q23.2a |
| inotropic GluR | | | | ✓ | □□□□ | ▪ | GRID2 | 4q22.1g-q22.2b |
| cGMP generation | CMPab | ✓ | | | ▪▪▪▪* | ▪ | GUCY1A2 | 11q22.3b-q22.3c |
| | | ✓ | | | ▪▪▪▪* | ▪ | GUCY1B3 | 4q32.1b |
| cAMP generation | CMPab | | ✓ | ✓ | ▪ | ▪ | ADCY4 | 14q12 |
| | | | ✓ | ✓ | ▪ | ▪▪ | ADCY8 | 8q24.2b |
| Guanylate | CMPab | | | ✓ | □□□□ | ▪ | DLG2 | 11q14.1d-q14.1e |
| kinases | | | | ✓ | □□□□ | ▪ | MAGI1 | 3p14.1d-p14.1c |
| Integrins | CMPab | ✓ | | | ▪▪▪▪* | ▪ | ITGB1 | 10p11.22b |
| | | ✓ | | | ▪▪▪▪* | ▪ | ITGB3 | 17q21.32a |
| | | ✓ | | | ▪▪▪▪* | ▪ | ITGB5 | 3q21.2a |
| | | ✓ | | | ▪▪▪▪* | ▪ | ITGB6 | 2q24.2b |
| | | ✓ | | | ▪▪▪▪* | ▪ | ITGAL | 16p11.2c |
| | | ✓ | | | ▪▪▪▪* | ▪ | ITGA2 | 5q11.2b |
| Matrix | CMPab | ✓ | | | ▪▪▪ | ▪ | MMP2 | 16q12.2c |
| metalloproteases | | ✓ | | | ▪▪▪ | ▪ | MMP15 | 16q13d |
| | | ✓ | | | ▪▪▪ | ▪ | MMP21 | 10q26.2a |
| | | ✓ | | | ▪▪▪ | ▪ | MMP24 | 20q11.22b |
| Scavenger | CMPab | ✓ | | | ▪▪▪▪* | ▪ | VLDLR | 9p24.2b |
| receptors | | | ✓ | | ▪▪▪▪* | ▪ | LRP1B | 2q22.1d-q22.2a |
| | | ✓ | ✓ | | ▪▪▪▪* | ▪ | LRP2 | 2q31.1a |
| | | ✓ | | | ▪▪▪▪* | ▪ | LRP8 | 1p32.3c |
| Synaptojanins | CMPab | ✓ | | | ▪▪▪▪ | ▪ | SYNJ1 | 21q22.11b |
| | | ✓ | | | ▪▪▪▪ | ▪ | SYNJ2 | 6q25.3d |
| Laminins | CMPab | | ✓ | | ▪▪▪▪* | ▪ | LAMA2 | 6q22.33d-q22.33e |
| | | | ✓ | | ▪▪▪▪* | ▪ | LAMA4 | 6q21i |
| Chromatin | CMPab | ✓ | | | ▪▪▪▪* | ▪ | CHD3 | 17p13.1d |
| remodelling | | ✓ | | | ▪▪▪▪* | ▪ | CHD5 | 1p36.31b |
| helicases | | | | | | | | |
| Forkhead | CMPab | ✓ | ✓ | | ▪▪▪▪ | ▪ | FOXA2 | 20p11.21c |
| transcription | | ✓ | ✓ | | ▪▪▪▪ | ▪ | FOXA3 | 19q13.32a |
| factors | | | | | | | | |
| transcription | CMPab | ✓ | | | ▪▪▪▪ | ▪ | RBPJ | 4p15.2b |
| factors | | ✓ | | | ▪▪▪▪ | ▪ | RBPJL | 20q13.12b |
| SIM2-like | CMPab | | ✓ | | ▪▪ | ▪ | NPAS3 | 14q13.1a-q13.1c |
| transcription | | | ✓ | | ▪▪ | ▪ | SIM2 | 21q22.13a |
| factors | | | | | | | | |
| RFX transcription | CMPab | | ✓ | | ▪▪ | ▪ | RFX2 | 19p13.3b |
| factors | | | ✓ | | ▪▪ | ▪ | RFX3 | 9p24.2b-p24.2a |
| Nuclear hormone | CMPab | | ✓ | | ▪▪ | ▪▪ | NR2F2 | 15q26.2c |
| transcription | | | ✓ | | ▪▪ | ▪▪ | RORA | 15q22.2a-q22.2b |
| factors | | | | | | | | |
| Exosome | CMPab | | ✓ | | ▪ | ▪ | EXOSC8 | 13q13.3b |
| components | | | ✓ | | ▪ | ▪ | EXOSC9 | 4q27c |
| Ca²⁺-activated | CMPab | ✓ | | | ▪▪▪▪ | ▪ | KCNN1 | 19p13.11d-p13.11c |
| potassium | | ✓ | | | ▪▪▪▪ | ▪ | KCNN4 | 19q13.31b |
| channels | | | | | | | | |
| Ras-like proteins | CMPab | ✓ | | | ▴ | ▪▪ | KRAS | 12p12.1b-p12.1a |
| | | ✓ | | | ▴ | ▪▪ | RAB4A | 1q42.13d |
| | | ✓ | | | ▴ | ▪▪ | RAB10 | 2p23.3b |
| | | ✓ | | | ▴ | ▪▪ | RAB18 | 10p12.1a |
| Tyrosine kinase | CMPab | ✓ | | | ▪▪▪ | ▪ | ERBB4 | 2q34c-q34e |
| receptors | | ✓ | | | ▪▪▪ | ▪ | IGF1R | 15q26.3a-q26.3b |
| | | ✓ | | | ▪▪▪ | ▪ | INSR | 19p13.2e |
| 14-3-3 proteins | CMPab | ✓ | | | ▪▪▪ | ▪ | NOV | 8q24.12b |
| | | ✓ | | | ▪▪▪ | ▪ | WISP1 | 8q24.22c |
| | | ✓ | | | ▪▪▪ | ▪ | WISP2 | 20q13.12a |
| | | ✓ | | | ▪▪▪ | ▪ | WISP3 | 6q21i |

Bolded genes are predicted independently by more than one method. Loci in bold have previously been associated with the disease.
Abbreviations.
Method: CMPab - CMP ab initio, CMPk - CMP known mode, CPSab - CPS ab initio, CPSk - CPS known mode.
Genetic support: HS ▪▪▪▪, MHS - ▪▪▪, MWS - ▪▪, WS - ▪.
Key to biological support (the present invention's scores): CMPab: ▪▪▪▪* - log χ² ≧ 9, ▪▪▪▪- 8 ≦ log χ² < 9, ▪▪▪- 7 ≦ log χ² < 8, ▪▪- 6 ≦ log χ² < 7, ▪- 5 ≦ log χ² < 6.
Lower χ² values considered for more genetically significant data based on statistics (≧ MWS) or proximity: □□□□- 4 ≦ log χ² < 5, □□□- 3 ≦ log χ² < 4. Lower χ² values considered for single domain proteins ▴- log χ² > 2.
CMPk: - Sc > 0.7, - Sc > 0.6, - Sc > 0.5, - Sc > 0.4, ∘- Sc > 0.25. CPS: ♦♦♦♦- p < 0.05 and Top 5, ♦♦♦- p < 0.05 and Top 10, ♦♦- Top 5, ♦- p < 0.05.
^{a}Includes known disease genes AGT and AGTR1.
^{b}1Mbp: CCKAR LTB4R CNR1 EDG3 GABRG3 GRIK2 GRIN2A NPY2R SSTR2 SSTR4 TACR1 GLP2R NTSR2 PARD3.
^{c}ADCY1 ADCY4 ADCY7 ADCY8 GUCY1A2 GUCY1A3 GUCY1B3 GUCY2D PRKACG PRKG1 CDC2 DRD1 GNAI3 GRM5 KRAS PDGFRA MAPK3 RAF1 SOS1 TJP1. TUBA1 TUBB2A TUBB4 TUBB2B

### Rheumatoid Arthritis (RA)

For the RA phenotype, CPS predicted up to 22 genes using known disease gene input mode; and up to 69 genes in ab initio input mode (Table 5). For known disease gene input mode, CMP predicted up to 17 genes. In ab initio input mode, the number of predictions was at most about 1569, with up to 41 genes reaching the arbitrary threshold χ2 max_unique (Table 7).

There were at most five known disease genes in the search spaces, and four were predicted through the different modules of the present invention. PTPN22, HLA-DRB1 and CIITA were predicted through CMP ab initio input mode, below the threshold cutoff. PTPN22 and HLA-DRB1 had a significance of χ2 min. HLA-DRB1, IL10 and CIITA share common pathways, but none were significant.

The regions on the genome with the highest association with the RA phenotype were known regions near the HLA-DRB1 (p=4.8×10-14), and within the known disease gene PTPN22 (p=8.8×10-11). More modest associations include regions around or within genes: IL2RA (p=7.0×10-6), IL2RB (p=7.9×10-6), GZMB (p=8.1×10-5), and in PRKCQ (p=5.6×10-5). CMP ab initio input mode predicted PRKCQ. CPS ab initio input mode predicted GZMB in top ranking and significant pathways. IL2RA and IL2RB were predicted through CPS ab initio input mode, sharing common pathways which were top ranking at the MHS and WS sets using the adjacent mapping and the BY mapping approaches.

In known disease gene input mode, the top ranking pathways were involved in the immune response. Using the nearest mapping approach, the top ranking significant pathways predicted were HLA-DQA and IL2RA, along with other cytokines and interleukins. The most significant pathway is “Th1/Th2 differentiation” for the adjacent and 1 Mbp mapping approaches, for the MHS, MWS and WS sets. The HS set instead has“Bystander B cell activation” was the most significant. CPS in ab initio input mode did not make any new predictions with the same pathways ranking top. However, the most significant pathway of the WS set using the 1 Mbp approach was “Apoptotic DNA fragmentation and tissue homeostasis” that implicates GZMB.

Predictions from CMP known disease gene input mode were mostly HLA genes, but similarity scores for the loci with the greater genetic support were between 0.3 and 0.4. Two runt-related transcription factors (RUNX2 and RUNX3) had similarity scores above 0.8 with the known disease gene RUNX1. RUNX2 influences joint formation through its regulation of osteoblast differentiation and RUNX3 is important in the development of basal root ganglia. An autoimmune function is also attributed to the RUNX gene family.

In CMP ab initio input mode, several themes were apparent: T-cell activation, actin cytoskeletal remodeling and loss of tissue differentiation. Protein kinase C are involved in TCR dependent T-cell activation. Antibodies against B1 integrin reduced resistance against delayed Fas-mediated apoptosis in T cells. Epithelial-mesenchymal transition (EMT) is a term applied to the process whereby cells undergo a switch from an epithelial phenotype with tight junctions, lateral, apical, and basal membranes, and lack of mobility into mesenchymal cells that have loose interactions with other cells, are non-polarized, motile and produce an extracellular matrix. EMT has been proposed to occur in RA.109 MAGI are tight junction proteins. Agents that elevate cAMP signaling may impair chondrocyte function in conditions such as arthritis.

Remodelling of the actin cytoskeleton in response to class 3 semaphorins.

TABLE 13
Top RA predictions made by CPS and CMP

| | | Mapping | | | | | | |
|---|---|---|---|---|---|---|---|---|
| | | Approach | | | Biological | Genetic | | |
| Group | Method | 1M | Adj | N | Support | support | Genes | Loci |
| Th1/Th2 | CPSab | ✓ | ✓ | | ♦♦♦♦ | ▪ | CD40 | 20q13.12b |
| Differentiation | CPSk | ✓ | ✓ | | ♦♦♦♦ | ▪▪▪▪ | HLA-DRA | 6p21.32b |
| | | ✓ | ✓ | | ♦♦♦♦ | ▪▪▪▪ | HLA-DRB1 | 6p21.32b |
| | | ✓ | ✓ | | ♦♦♦♦ | ▪▪▪ | IFNGR1 | 6q23.3c |
| | | ✓ | ✓ | | ♦♦♦♦ | ▪ | IFNGR2 | 21q22.11c |
| | | ✓ | ✓ | ✓ | ♦♦♦♦ | ▪▪▪ | IL2RA | 10p15.1b-p15.1a |
| | | ✓ | ✓ | | ♦♦♦♦ | ▪ | PVRL1 | 11q23.3f |
| | | ✓ | ✓ | ✓ | ♦♦♦♦ | ▪▪ | IL18R1 | 2q12.1a |
| Apoptotic | CPSab | ✓ | | | ♦ | ▪ | CASP3 | 4q35.1e |
| DNA | | ✓ | | | ♦ | ▪ | CASP7 | 10q25.3a |
| fragmentation | | ✓ | | | ♦ | ▪▪ | DFFB | 1p36.32b |
| and tissue | | ✓ | ✓ | | ♦ | ▪▪ | GZMB | 14q12a |
| homeostasis | | ✓ | | | ♦ | ▪ | HMGB1 | 13q12.3c |
| | | ✓ | | | ♦ | ▪▪ | TOP2A | 17q21.2a |
| HLA | CMPk | ✓ | ✓ | ✓ | ∘ | ▪▪▪▪ | HLA-DQA1 | 6p21.32b |
| | | ✓ | | | | ▪▪▪▪ | HLA-DRB5 | 6p21.32b |
| | | ✓ | | | | ▪▪▪▪ | HLA-DPB1 | 6p21.32b |
| Runt-related | CMPk | | ✓ | | | ▪ | RUNX2 | 6p12.3f |
| transcription | | ✓ | | | | ▪ | RUNX3 | 1p36.11c |
| factors | | | | | | | | |
| Protein kinase C | CMPab | ✓ | | | ▪▪ | ▪▪▪ | PRKCQ | 10p15.1a |
| TCR | | ✓ | | | ▪▪ | ▪▪▪ | PRKCZ | 1p36.33a |
| dependent T- | | | | | | | | |
| cell activation | | | | | | | | |
| integrins | CMPab | ✓ | | ✓ | ▪▪▪▪ | ▪▪ | ITGB1 | 10p11.22b |
| | | ✓ | | ✓ | ▪▪▪▪ | ▪▪ | ITGB3 | 17q21.32a |
| Tight junctions | CMPab | ✓ | ✓ | ✓ | ▪▪ | ▪▪ | MAGI1 | 3p14.1d-p14.1c |
| Guanylate | | ✓ | ✓ | ✓ | ▪▪ | ▪▪ | MAGI3 | 1p13.2c-p13.2b |
| kinases | | | | | | | | |
| Ca²⁺-triggered | CMPab | ✓ | | | ▪▪▪ | ▪ | OTOF | 2p23.3b |
| synaptic | | ✓ | | | ▪▪▪ | ▪ | FER1L6 | 8q24.13c |
| vesicle- | | | | | | | | |
| plasma | | | | | | | | |
| membrane | | | | | | | | |
| fusion | | | | | | | | |
| cAMP-gated | CMPab | ✓ | ✓ | ✓ | ▪▪▪ | ▪ | HCN1 | 5p12a |
| potassium | | ✓ | ✓ | ✓ | ▪▪▪ | ▪ | HCN4 | 15q24.1a |
| channels | | | | | | | | |
| vitamin D- | CMPab | ✓ | | | ▪ | ▪ | SMARCA2 | 9p24.3a |
| coupled and | | ✓ | | | ▪ | ▪ | CHD7 | 8q12.2a |
| other | | | | | | | | |
| transcription | | | | | | | | |
| regulation | | | | | | | | |
| | CMPab | | | | ▪▪▪ | ▪ | DNAJA2 | 16q12.1a |
| | | | | | ▪▪▪ | ▪ | DNAJA4 | 15q25.1a |
| Clathrin- | CMPab | ✓ | | | ▪▪▪▪ | ▪ | GGA1 | 22q13.1a |
| mediated | | ✓ | | | ▪▪▪▪ | ▪ | GGA2 | 16p12.1c |
| endocytosis | | | | | | | | |
| Inhibitory | CMPab | | ✓ | ✓ | ▪▪▪ | ▪ | GRM4 | 6p21.31f-p21.31e |
| Metabotropic | | | ✓ | ✓ | ▪▪▪ | ▪ | GRM7 | 3p26.1b-p26.1a |
| Glu receptors | | | | | | | | |
| ECM | CMPab | | ✓ | ✓ | ▪▪▪▪* | ▪ | ADAMTS6 | 5q12.3a-q12.3b |
| remodelling | | | ✓ | ✓ | ▪▪▪▪* | ▪ | ADAMTS18 | 16q23.1c |
| | | | ✓ | ✓ | ▪▪▪▪ | ▪ | ADAMTS20 | 12q12f |
| | | | ✓ | ✓ | ▪▪▪ | | ADAMTSL2 | 9q34.2a |
| Actin | CMPab | | ✓ | ✓ | ▪▪▪▪* | ▪ | FARP2 | 2q37.3f |
| cytoskeletal | | | ✓ | ✓ | ▪▪▪▪* | ▪ | EPB41L4A | 5q22.2a |
| remodelling | | | | | | | | |
| ankyrins | CMPab | ✓ | | | ▪▪ | ▪ | ANK1 | 8p11.21b |
| | | ✓ | | | ▪▪ | ▪ | ANK2 | 4q26a |
| | | ✓ | | | ▪▪ | ▪ | ANK3 | 10q21.2a |
| Cell-ECM | CMPab | | | ✓ | ▪▪ | ▪ | LRP1B | 2q22.1d-q22.2a |
| interactions | | | | ✓ | ▪▪ | ▪ | NID2 | 14q22.1d |

Bolded genes are predicted independently by more than one method. Loci in bold have previously been associated with the disease.
Abbreviations.
Method: CMPab- CMP ab initio, CMPk- CMP known mode, CPSab- CPS ab initio, CPSk- CPS known mode.
Genetic support: HS ▪▪▪▪, MHS-▪▪▪, MWS-▪▪, WS-▪.
Key to biological support (the present invention's scores): CMPab: ▪▪▪▪*-log χ² ≧ 9, ▪▪▪▪-8 ≦ log χ² < 9, ▪▪▪-7 ≦ log χ² < 8, ▪▪-6 ≦ log χ² < 7, ▪-5 ≦ log χ² < 6.
Lower χ² values considered for more genetically significant data based on statistics (≧MWS) or proximity: □□□□- 4 ≦ log χ² < 5, □□□- 3 ≦ log χ² < 4.
Lower χ² values considered for single domain proteins ▴- log χ² > 2.
CMPk: -Sc > 0.7, -Sc > 0.6, -Sc > 0.5, -Sc > 0.4, ∘-Sc > 0.25.
CPS: ♦♦♦♦-p < 0.05 and Top 5, ♦♦♦-p < 0.05 and Top 10, ♦♦-Top 5, ♦-p < 0.05.

Type I diabetes (T1D)

For the T1D phenotype, CPS predicted up to 23 genes using known disease gene input mode; and up to 133 genes in ab initio input mode (Table 5). For known disease gene input mode, CMP predicted up to 23 genes. In ab initio input mode, the number of predictions was at most about 1606, with up to 71 genes reaching the arbitrary threshold χ2 max_unique (Table 7).

Ten genes from OMIM were known disease genes for the T1D phenotype, and at most 6 were in the gene search spaces following the SNP to gene mappings. Of these, CPS in known disease gene input mode predicted IL2RA and CCR5, both in the common pathway “Cytokine-cytokine receptor interaction” with the known disease gene IL6. IL2RA also shares two other pathways with IL6: “Hematopoietic cell lineage” and “Jak-STAT signaling pathway”. CPS ab initio input mode predicted CTLA4 through “The Co-Stimulatory Signal During T-cell Activation” pathway. CMP ab initio input mode predicted IL2RA, PTPN22, CTLA4 and CCR5, but they all fail to reach the χ2 max_unique threshold.

The known loci that had relatively strong association signals in the WTCCC study were the MHC locus (p=2.42×10⁻¹³⁴), PTPN22 (p=1.95×10⁻¹³), around IL2RA/CD25 (p=7.97×10-6) and CTLA4 (p=3.27×10-5). Novel regions of association include two regions on chromosome 12 that harbor genes ERBB3, SH2B3, TRAFD1 and PTPN11 as potential candidates (12q13,p=1.14×10-11; 12q24, p=2.17×10-15). Weaker associations on chromosome 12 are near CD69 and CLEC (p=1.02×10-4). PTPN2 is located near a region of modest association on chromosome 18 (18 p11, p=1.89×10-6). The 12q24 locus and the 18 p11 locus also feature prominently in the CD and RA phenotypes, indicative of important autoimmune susceptibility regions. Further region of modest association (4q27, p=5.01×10-7) are near genes 1 IL2 and IL21. CMP known predicts PTPN11 and PTPN2 as they share a common domain with PTPN22. CPS ab initio input mode predicted IL2, IL2RA, and PTPN11 through the “Jak-STAT signaling pathway” they share.

The top ranking CPS known pathway implicated by the present innovation using the nearest mapping approach were the “Jak-STAT signaling pathway” as aforementioned. The most significant pathways were related to IL2 signaling and T-cell activation. Expanding to the adjacent mapping, the top ranking pathway for the MWS and WS sets was the “Cytokine8 cytokine receptor interaction” pathway which predicted the chemokine receptors with the CC motif along with the IL2 receptors and interleukins. In this mapping, the pathways with statistically significant enrichment for genes were the IL2 pathways as in the nearest mapping. Similarly, the larger 1 Mbp BY mapping were the chemokine intereactions as a top ranking. The most enriched pathway interestingly was the “Selective expression of chemokine receptors during T-cell polarization”. CPS ab initio input mode produced resulted similar to the known disease gene input mode results, with IL2 receptor and signaling pathways featuring prominently.

The highest scoring CMP prediction was CCR2 (0.8) with the known disease gene CCR5. This chemokine has been associated with insulin dependent diabetes. PTPN11 and PTPN2 have relatively low similarity scores with PTPN22. Numerous FOX genes were predicted, with similarity scores around 0.4.

The T1D CMP ab initio input mode predicted results related to the immune system with MHC_I and MHC_II molecules and multiple butyrophilins, and histones. Interestingly, it was the only one of the seven phenotypes where RNA-mediated gene silencing was implicated. A distinct butyrophilins locus BTN3A2 was recently associated with T1D. Butyrophilins alter T-cell responsiveness. An increase of cathepsin D activity was found in serum of diabetic patients compared to controls. For single domain proteins, histones and H1 linker histones had high scores. DNA is wound round the core histones H2, H3 and H4 and clipped in place with the linker histones H1 and H5. However, linker histones are not always sequestered in the nucleus and can be transported around the cell and also have been found in macrophage granules and other immune cells. In particular, H1 histones can replace the more repressive H5 histones in chromatin, remodeling heterochromatin to a more open euchromatin structure. Histones are also present on the cell surface of apoptotic cells and could be involved in provoking autoimmune responses. Ephrins involved in both diabetes phenotypes. SYNGAP1 and RASA1 are inhibitory regulators of the Ras-cAMP pathway, possibly involved in membrane trafficking. Eph receptors and their ephrin ligands coordinate chemotactic cell-positioning programs, modulating cell motility to control cell-cell repulsion or adhesion.

TABLE 14
Top T1D predictions made by CPS and CMP

| | | Mapping | | | | | | |
|---|---|---|---|---|---|---|---|---|
| | | Approach | | | Biological | Genetic | | |
| Group | Method | 1M | Adj | N | Support | support | Genes | Loci |
| Jak-STAT | CPSk | ✓ | ✓ | ✓ | ♦♦♦♦ | ▪▪▪ | IL2 | 4q27d |
| signaling | | ✓ | ✓ | ✓ | ♦♦♦♦ | ▪▪▪ | IL2RA | 10p15.1b-p15.1a |
| pathway^{b} | | ✓ | ✓ | ✓ | ♦♦♦♦ | ▪▪ | IL2RB | 22q12.3d |
| | | ✓ | ✓ | ✓ | ♦♦♦♦ | ▪▪▪ | PTPN11 | 12q24.13a |
| | | ✓ | ✓ | ✓ | ♦♦♦♦ | ▪ | STAT3 | 17q21.2b |
| | | ✓ | ✓ | ✓ | ♦♦♦♦ | ▪ | STAT4 | 2q32.3a |
| | | ✓ | ✓ | ✓ | ♦♦♦♦ | ▪▪▪ | SOCS1 | 16p13.13c |
| | | ✓ | ✓ | ✓ | ♦♦♦♦ | ▪▪ | IL21 | 4q27d |
| | | | ✓ | | ♦♦ | ▪▪ | IL5RA | 3p26.3a |
| | | ✓ | ✓ | | ♦♦♦♦ | ▪▪ | IL7R | 5p13.2c |
| | | ✓ | ✓ | | ♦♦♦♦ | ▪▪ | IL10RA | 11q23.3c |
| | | ✓ | ✓ | | ♦♦ | ▪ | STAT5A | 17q21.2b |
| | | ✓ | ✓ | | ♦♦ | ▪ | STAM | 10p12.33c |
| Selective | CPSk | ✓ | ✓ | | ♦♦♦ | ▪▪ | CD28 | 2q33.2a |
| expression of | | ✓ | ✓ | | ♦♦♦♦ | ▪▪ | CCR1 | 3q21.31i |
| chemokine | | ✓ | ✓ | ✓ | ♦♦♦♦ | ▪▪ | CCR3 | 3p21.31i |
| receptors during | | ✓ | | | ♦♦♦ | ▪ | CCR4 | 3p22.3c |
| T-cell polarization | | ✓ | | | ♦♦♦ | ▪▪ | CCR5 | 3p21.31i |
| | | ✓ | ✓ | | ♦♦♦♦ | ▪▪ | CCR7 | 17q21.2a |
| | | ✓ | ✓ | ✓ | ♦♦♦♦ | ▪▪▪ | IL2 | 4q27d |
| | | ✓ | | | ♦♦♦ | ▪ | IL12RB2 | 1p31.3a |
| | | ✓ | ✓ | | ♦♦♦ | ▪▪ | CCL3 | 17q12b |
| | | ✓ | | | ♦♦♦ | ▪ | CCL4 | 17q12b |
| Chemokine (CC | CMPk | ✓ | ✓ | | | ▪▪ | CCR1 | 3p21.31i |
| motif) receptors | | ✓ | ✓ | | | ▪▪ | CCR2 | 3p21.31i |
| | | ✓ | ✓ | | | ▪▪ | CCR4 | 3p22.3c |
| | | ✓ | ✓ | ✓ | | ▪▪ | CCR3 | 3p21.31i |
| | | ✓ | ✓ | | ∘ | ▪▪ | CCR7 | 17q21.2a |
| | | ✓ | ✓ | | ∘ | ▪▪ | CCR9 | 3p21.31j-p21.31i |
| Protein tyrosine | CMPk | ✓ | ✓ | ✓ | ∘ | ▪▪▪ | PTPN2 | 18p11.21d |
| phosphatases, | | ✓ | ✓ | ✓ | ∘ | ▪▪▪ | PTPN11 | 12q24.13a |
| non-receptor | | | | | | | | |
| butyrophilins | CMPab | ✓ | | | ▪▪ | ▪▪▪ | BTN1A1 | 6p22.1d |
| | | ✓ | | | ▪▪ | ▪▪▪ | BTN2A2 | 6p22.1d |
| | | ✓ | | ✓ | □□□□ | ▪▪▪ | BTN2A1 | 6p22.1d |
| | | ✓ | | | □□□□ | ▪▪▪ | BTN2A3 | 6p22.1d |
| | | ✓ | | | □□□□ | ▪▪▪ | BTN3A1 | 6p22.1d |
| | | ✓ | | | □□□□ | ▪▪▪ | BTN3A3 | 6p22.1d |
| | | ✓ | | ✓ | □□□□ | ▪▪▪ | BTNL2 | 6p21.32b |
| | | ✓ | | | □□□□ | ▪▪ | LOC391037 | 1p33c |
| Krab/SCAN C₂H₂ | CMPab | ✓ | ✓ | | ▪ | ▪▪▪ | ZNF192 | 6p22.1b |
| Zn fingers | | ✓ | ✓ | | ▪ | ▪▪▪ | ZKSCAN3 | 6p22.1b |
| | | ✓ | ✓ | | ▪ | ▪▪▪ | ZKSCAN4 | 6p22.1b |
| PI3 kinases | CMPab | ✓ | | | ▪▪▪▪* | ▪ | PIK3C2A | 11p15.1e |
| | | ✓ | ✓ | ✓ | ▪▪▪▪* | ▪ | PIK3C2B | 1q32.1f |
| | | ✓ | ✓ | ✓ | ▪▪▪▪* | ▪ | PIK3C2G | 12p12.3b |
| | | ✓ | | | ▪▪▪▪* | ▪ | PIK3CB | 3q22.3c |
| Aspartic | CMPab | ✓ | | | □□□□ | ▪▪ | CTSD | 11p15.5b |
| proteases | | ✓ | | | □□□□ | ▪▪ | REN | 1q32.1f |
| M28 Zinc | CMPab | ✓ | | | ▪▪▪▪* | ▪ | TFR2 | 7q22.1c |
| metallopeptidases | | ✓ | | | ▪▪▪▪* | ▪ | NAALAD2 | 11q14.3b |
| ADAMTS | CMPab | ✓ | | | ▪▪▪▪* | ▪ | ADAMTS1 | 21q21.3a |
| proteases | | ✓ | | | ▪▪▪▪* | ▪ | ADAMTS2 | 5q35.3d |
| | | ✓ | | | ▪▪▪▪* | ▪ | ADAMTS5 | 21q21.3a |
| | | ✓ | | | ▪▪▪▪* | ▪ | ADAMTS7 | 15q25.1a |
| | | ✓ | | | ▪▪▪▪* | ▪ | ADAMTS17 | 15q26.3c |
| | | ✓ | | | ▪▪▪▪* | ▪ | ADAMTS18 | 16q23.1c |
| Matrix | CMPab | ✓ | | | ▪▪▪▪ | ▪ | MMP8 | 11q22.2b |
| metalloproteases | | ✓ | | | ▪▪▪▪ | ▪ | MMP14 | 14q11.2f |
| | | ✓ | | | ▪▪▪▪ | ▪ | MMP19 | 12q13.2c |
| | | ✓ | | | ▪▪▪▪ | ▪ | MMP20 | 11q22.2a-q22.2b |
| | | ✓ | | | ▪▪▪▪ | ▪ | MMP27 | 11q22.2b |
| | | ✓ | | | ▪▪▪▪ | ▪ | MMP28 | 17q12b |
| Notch proteins | CMPab | ✓ | | | ▪▪▪▪* | ▪▪ | NOTCH2 | 1p12a |
| | | ✓ | | | ▪▪▪▪* | ▪▪ | NOTCH4 | 6p21.32b |
| Argonaut RNAi- | CMPab | ✓ | | | ▪▪▪▪ | ▪ | EIF2C3 | 1p34.3d |
| mediated gene | | ✓ | | | ▪▪▪▪ | ▪ | EIF2C4 | 1p34.3e |
| silencing | | ✓ | | | ▪▪▪▪ | ▪ | EIF2C1 | 1p34.3e-p34.3d |
| STATs | CMPab | ✓ | | | ▪▪▪▪* | ▪ | STAT1 | 2q32.2b |
| | | ✓ | | | ▪▪▪▪* | ▪ | STAT2 | 12q13.2c |
| | | ✓ | ✓ | ✓ | ▪▪▪▪* | ▪ | STAT3 | 17q21.2b |
| | | ✓ | ✓ | ✓ | ▪▪▪▪* | ▪ | STAT4 | 2q32.3a |
| | | ✓ | ✓ | | ▪▪▪▪* | ▪ | STAT5A | 17q21.2b |
| | | | | | | | &/or | |
| | | ✓ | | | ▪▪▪▪* | ▪ | STAT5B | 2q32.2b |
| Linker_Histone | CMPab | ✓ | | | ▴ | ▪▪▪▪ | HIST1H1B | 6p22.1c |
| | | ✓ | ✓ | | ▴ | ▪▪▪▪ | HIST1H1A | 6p22.1d |
| | | ✓ | ✓ | | ▴ | ▪▪▪▪ | HIST1H1C | 6p22.1d |
| | | ✓ | ✓ | | ▴ | ▪▪▪▪ | HIST1H1D | 6p22.1d |
| | | ✓ | ✓ | | ▴ | ▪▪▪▪ | HIST1H1E | 6p22.1d |
| | | ✓ | | | ▴ | ▪▪▪▪ | HIST1H1T | 6p22.1d |
| Histones | CMPab | ✓ | ✓ | ✓ | ▴ | ▪▪▪▪ | HIST1H2A* | 6p22 |
| | | ✓ | ✓ | ✓ | ▴ | ▪▪▪▪ | HIST1H2B* | 6p22 |
| | | ✓ | ✓ | ✓ | ▴ | ▪▪▪▪ | H3F3A | 1q42.12c |
| | | ✓ | ✓ | ✓ | ▴ | ▪▪▪▪ | HIST1H3* | 6p22 |
| | | ✓ | ✓ | ✓ | ▴ | ▪▪▪▪ | HIST1H4* | 6p22 |
| MHC II α subunits | CMPab | ✓ | | | ▴ | ▪▪▪▪ | HLA-DMA | 6p21.32a |
| | | ✓ | | | ▴ | ▪▪▪▪ | HLA-DOA | 6p21.32a |
| | | ✓ | | | ▴ | ▪▪▪▪ | HLA-DPA1 | 6p21.32a |
| | | ✓ | ✓ | ✓ | ▴ | ▪▪▪▪ | HLA-DQA1 | 6p21.32b |
| | | ✓ | | | ▴ | ▪▪▪▪ | HLA-DQA2 | 6p21.32a |
| | | ✓ | ✓ | | ▴ | ▪▪▪▪ | HLA-DRA | 6p21.32b |
| MHC II β subunits | CMPab | ✓ | | | ▴ | ▪▪▪▪ | HLA-DMB | 6p21.32a |
| | | ✓ | | | ▴ | ▪▪▪▪ | HLA-DOB | 6p21.32a |
| | | ✓ | | | ▴ | ▪▪▪▪ | HLA-DPB1 | 6p21.32b |
| | | ✓ | ✓ | | ▴ | ▪▪▪▪ | HLA-DQB1 | 6p21.32a |
| | | ✓ | | | ▴ | ▪▪▪▪ | HLA-DQB2 | 6p21.32b |
| | | ✓ | ✓ | | ▴ | ▪▪▪▪ | HLA-DRB1 | 6p21.32b |
| | | ✓ | | | ▴ | ▪▪▪▪ | HLA-DRB5 | 6p21.32a |
| MHC I | CMPab | | ✓ | | □□□□ | ▪ | AZGP1 | 7q22.1b |
| | | ✓ | ✓ | ✓ | ▪ | ▪▪▪▪ | HFE | 6p22.1d |
| | | ✓ | | | □□□□ | ▪▪▪▪ | HLA-B | 6p21.33a |
| | | ✓ | | | □□□□ | ▪▪▪▪ | HLA-C | 6p21.33a |
| | | ✓ | ✓ | | ▪ | ▪▪▪▪ | MICA | 6p21.33a |
| | | ✓ | | | □□□□ | ▪▪▪▪ | MICB | 6p21.33a |
| Contactin-like cell | CMPab | | | ✓ | ▪ | ▪ | CNTN1 | 12q12c-q12d |
| adhesion | | | | ✓ | ▪ | ▪ | CNTN4 | 3p26.3b-p26.3a |
| molecules | | | | ✓ | ▪ | ▪ | DSCAML1 | 11q23.3c |
| | | | | ✓ | ▪ | ▪ | SDK1 | 7p22.2b-p22.2a |
| Cadherins | CMPab | | ✓ | | □□□□ | ▪ | CDH4 | 20q13.33b-q13.33c |
| | | | ✓ | | □□□□ | ▪ | CDH5 | 16q21e |
| | | | ✓ | | □□□□ | ▪ | CDH7 | 18q22.1c |
| | | | ✓ | | □□□□ | ▪ | CDH8 | 16q21c |
| | | | ✓ | | □□□□ | ▪ | CDH9 | 5p14.1c |
| | | | ✓ | | □□□□ | ▪ | CDH18 | 5p14.3d |
| | | | ✓ | | □□□□ | ▪ | CDH19 | 18q22.1c-q22.1d |
| | | | ✓ | | □□□□ | ▪ | CDH20 | 18q21.33a |
| | CMPab | ✓ | | | □□□□ | ▪▪▪ | SYNGAP | 6p21.32a |
| | | ✓ | | | □□□□ | ▪▪▪ | RASA1 | 5q14.3d |
| | | | | | | | RASAL1 | 12q24.13b |

Bolded genes are predicted independently by more than one method. Loci in bold have previously been associated with the disease.
Abbreviations.
Method: CMPab- CMP ab initio, CMPk- CMP known mode, CPSab- CPS ab initio, CPSk- CPS known mode.
Genetic support: HS ▪▪▪▪, MHS-▪▪▪, MWS-▪▪, WS-▪.
Key to biological support (CPS and CMP scores): CMPab: ▪▪▪▪*-log χ² ≧ 9, ▪▪▪▪-8 ≦ log χ² < 9, ▪▪▪-7 ≦ log χ² < 8, ▪▪-6 ≦ log χ² < 7, ▪-5 ≦ log χ² < 6.
Lower χ² values considered for more genetically significant data based on statistics (≧ MWS) or proximity: □□□□- 4 ≦ log χ² < 5, □□□- 3 ≦ log χ² < 4.
Lower χ² values considered for single domain proteins ▴ - log χ² > 2.
CMPk: - Sc > 0.7, - Sc > 0.6, - Sc > 0.5, - Sc > 0.4, ∘- Sc > 0.25.
CPS: ♦♦♦♦- p < 0.05 and Top 5, ♦♦♦- p < 0.05 and Top 10, ♦♦- Top 5, ♦- p < 0.05.
^{a}HIST1H2AA, HIST1H2AB, HIST1H2AC, HIST1H2AD, HIST1H2AE, HIST1H2AG, HIST1H2AH HIST1H2AI, HIST1H2AJ, HIST1H2AK, HIST1H2AL, HIST1H2AM, HIST1H2AA, HIST1H2BA, HIST1H2BB, HIST1H2BC, HIST1H2BD, HIST1H2BE, HIST1H2BF, HIST1H2BG, HIST1H2BH, HIST1H2BI, HIST1H2BJ, HIST1H2BK, HIST1H2BM, HIST1H2BN, HIST1H2BO, HISTH3A, HISTH3B, HISTH3C, HISTH3D, HISTH3E, HISTH3F, HISTH3G, HISTH3H, HISTH3I, HISTH3J, HISTH4A, HISTH4B, HISTH4C, HISTH4D, HISTH4E, HIST4F, HISTH4G
^{b}CNTFR, CSF2RB, IL11RA, IL12RB2, IL15RA, PIK3CB, SOS2, STAT1, STAT2, STAT5B, PIK3R3, ISGF3G, IL23A, IL23R, SPRED1

### Type II Diabetes (T2D)

CPS predicted up to 52 genes using known disease gene input mode and up to 104 genes for ab initio input mode depending on the statistical significance of the SNP set used and the mapping approach adopted (Table 5). Up to 24 pathways reached statistical significance in the WS search space using the 0.5 Mbp BY mapping approach. CMP using known disease gene input mode predicted up to 88 genes while the ab initio input mode method predicted at most about 1178 genes, with about 139 over the χ2 max_unique threshold (Table 7). Top predictions for T2D are shown in Table 5.

Genes previously associated with type II diabetes were insulin related, involve sugar metabolism, lipid or fatty acid metabolism, lipid transport, hormone signaling and pancreatic beta cell related functions. Thirty genes from OMIM were collected using known disease gene input mode for the T2D phenotype, and 5 were in the gene search spaces following the SNP to gene mappings. CPS predicted AKT2 since it is part of the adipocytokine signaling pathway along with known disease genes SLC2A4, IRS1 and IRS2. AKT2 were also a component of the more extensive insulin signaling pathway that included the latter genes along with GCK and PTPN1. CMP predicted TCF2 as it shares common domains with known disease gene TCF7L2. TCF7L2 itself was also predicted numerous times through both CPS ab initio input mode and is a part of multiple pathways.

The WTCCC study detected a widely replicated association with transcription factor TCF7L2 (p=5.68×10-13). Novel loci implicated FTO (p=5.24×10-8)—a fat-mass and obesity gene; and CDKAL1 (p=1.02×10-6), a gene now known to be implicated in pancreatic β-cell function. A cluster of SNPs with modest association (p values between 10-4 and 10-5) was found near genes HHEX and IDE, which recent studies have implicated in type II diabetes. Of these genes, CMP predicted HHEX as it has a homeobox domain in common with known disease genes IPF1, PAX4, TCF1 and TCF2. As aforementioned, TCF7L2 was in multiple pathways with known disease gene input mode.

TABLE 15
Top T2D predictions made by CPS and CMP

| | | Mapping | | | | | | |
|---|---|---|---|---|---|---|---|---|
| | | Approach | | | Biological | Genetic | | |
| Group | Method | 1M | Adj | N | Support | Support | Genes | Loci |
| Maturity onset | CPSk | ✓ | ✓ | ✓ | ♦♦♦♦ | ▪▪▪ | HHEX | 10q23.33a |
| diabetes of the | | ✓ | ✓ | ✓ | ♦♦ | ▪ | NR5A2 | 1q32.1a |
| young | | | | | | | | |
| Ca²⁺-binding | CMPk | ✓ | | | | ▪▪ | DUOX1 | 15q21.1a |
| | | ✓ | | | | ▪▪ | KCNIP2 | 10q24.32a |
| Homeobox | CMPk | ✓ | ✓ | ✓ | | ▪▪▪ | HHEX | 10q23.33a |
| transcription | | ✓ | ✓ | | | ▪▪ | PITX3 | 10q24.32b |
| factors | | ✓ | | | | ▪ | VSX1 | 20p11.21a |
| | | ✓ | ✓ | ✓ | | ▪ | BARX2 | 11q24.3b |
| HLH | CMPk | ✓ | ✓ | | | ▪▪ | HAND1 | 5q33.2b |
| transcription | | ✓ | ✓ | ✓ | | ▪▪ | NEUROG1 | 5q31.1f |
| factors | | | | | | | | |
| Hormone | CMPk | ✓ | ✓ | | | ▪▪ | PPARA | 22q13.31d |
| receptor | | ✓ | | | | ▪ | PPARD | 6p21.31c |
| transcription | | | | | | | | |
| factors | | | | | | | | |
| Sugar | CMPk | ✓ | ✓ | ✓ | | ▪▪ | SLC2A1 | 1p34.2a |
| transporters | | ✓ | ✓ | ✓ | | ▪▪ | SLC2A3 | 12p13.31c |
| | | ✓ | | | | ▪▪ | SLC2A14 | 12p13.31c |
| ROS generators | CMPab | ✓ | | | ▪▪▪▪* | ▪ | DUOX1 | 15q21.1a |
| | | ✓ | | | ▪▪▪▪* | ▪ | DUOX2 | 15q21.1a |
| | | ✓ | | | ▪▪▪▪* | ▪ | NOX5 | 15q23a |
| Phospholipases | CMPab | ✓ | | | ▪▪▪▪* | ▪ | PLCB2 | 15q15.1a |
| | | ✓ | | | ▪▪▪▪* | ▪ | PLCD1 | 3p22.2a |
| | | ✓ | | | ▪▪▪▪* | ▪ | PLCD3 | 17q21.31d |
| ADAM | CMPab | ✓ | ✓ | ✓ | ▪▪▪▪* | ▪ | ADAMTS3 | 4q13.3c |
| metalloproteases | | ✓ | | | ▪▪▪▪* | ▪ | ADAMTS5 | 21q21.3a |
| | | ✓ | ✓ | | ▪▪▪▪* | ▪ | ADAMTS16 | 5p15.32b-p15.32a |
| | | ✓ | | | ▪▪▪▪* | ▪ | ADAM11 | 17q21.31c |
| | | ✓ | | | ▪▪▪▪* | ▪ | ADAM28 | 8p21.2d |
| Chromatin | CMPab | ✓ | | | ▪▪▪▪* | ▪ | CHD6 | 20q12c |
| remodelling | | ✓ | | | ▪▪▪▪* | ▪ | CHD7 | 8q12.2a |
| helicases | | ✓ | | | ▪▪▪▪* | ▪ | CHD9 | 16q12.2a |
| Mitochondrial | CMPab | ✓ | | | ▪▪▪▪* | ▪ | IVD | 15q15.1a |
| branched chain | | ✓ | | | ▪▪▪▪* | ▪ | ACAD8 | 11q25e |
| amino acid and | | ✓ | | | ▪▪▪▪* | ▪ | ACAD9 | 3q21.3c |
| fatty acid | | | | | | | | |
| catabolism | | | | | | | | |
| Regulators of | CMPab | | ✓ | | ▪▪▪ | ▪ | BAI1 | 8q24.3e |
| membrane | | | ✓ | | ▪▪▪ | ▪▪ | CELSR1 | 22q13.31d |
| dynamics | | | ✓ | | ▪▪▪ | ▪ | LPHN2 | 1p31.1b |
| Centromere- | CMPab | ✓ | | | ▪▪▪▪ | ▪ | JRK | 8q24.3e |
| binding proteins | | ✓ | | | ▪▪▪▪ | ▪ | TIGD3 | 11q13.1c |
| | | | | | | | TIGD6 | 5q33.1c |

Bolded genes are predicted independently by more than one method. Loci in bold have previously been associated with the disease.
Abbreviations.
Method: CMPab- CMP ab initio, CMPk- CMP known mode, CPSab- CPS ab initio, CPSk- CPS known mode.
Genetic support: HS ▪▪▪▪, MHS-▪▪▪, MWS-▪▪, WS-▪.
Key to biological support (present invention's scores): CMPab: ▪▪▪▪*-log χ² ≧ 9, ▪▪▪▪-8 ≦ log χ² < 9, ▪▪▪-7 ≦ log χ² < 8, ▪▪-6 ≦ log χ² < 7, ▪-5 ≦ log χ² < 6.
Lower χ² values considered for more genetically significant data based on statistics (≧MWS) or proximity: □□□□- 4 ≦ log χ² < 5, □□□- 3 ≦ log χ² < 4.
Lower χ² values considered for single domain proteins ▴ - log χ² > 2.
CMPk: - Sc > 0.7, - Sc > 0.6, - Sc > 0.5, - Sc > 0.4, ∘- Sc > 0.25.
CPS: ♦♦♦♦- p < 0.05 and Top 5, ♦♦♦- p < 0.05 and Top 10, ♦♦—Top 5, ♦- p < 0.05.

Using known disease gene input mode, the most common pathways predicted by CPS varied. Cancer pathways were implicated by transcription factors in the known disease genes, using both the NN and BY mapping approaches. “Maturity onset diabetes of the young” was significant or top ranking in the MHS, MWS and WS sets using the nearest NN approach, further implicating HHEX. The CPS ab initio input modes predicted varied depending on both the mapping approach and the significance level threshold.

CMP predictions were based on known disease gene input mode transcription factors, sugar transport and calcium handling (Table 16). The candidate gene with the highest similarity score to a known disease gene in the MHS SNP dataset was HHEX which had a similarity score of 0.571 with the known disease gene IPF1. The present inventors searched for higher scoring genes in the WS and MWS datasets and PPARA emerged as a strong biological candidate but also had good genetic support, being implicated by 20 weakly significant SNPs. The calcium handling theme was also predicted by CMP ab initio input mode, where domain included EF-hand domains in the phospholipases, and Ca²⁺-binding EGF domains in SCUBE genes and Toll-like proteins were predicted. In addition, CMP ab initio input mode provided some interesting candidates on the T2D phenotype. Candidates involved with redox reactions feature prominently among predictions: NFKB is a known player in transcriptional activation of the oxidative stress response. Candidates include enzymes that generate reactive oxygen species such as the peroxide-generating DUOX genes, which complement the nitric oxide-generating known disease gene NOX5. A group of mitochondrial enzymes involved in branched chain amino acid catabolism are also predicted. Like the DUOX-genes, they utilize FAD as an electron source for redox reactions. IVD catabolizes leucine, ACAD8 catabolizes valine and ACAD9 catabolizes long chain fatty acids. Two of these mitochondrial genes are common to other phenotypes and will be discussed in detail later.

TABLE 16
T2D CMP known results

| | | | | | Nearest | | | | | |
|---|---|---|---|---|---|---|---|---|---|---|
| | | Known | | Common | MHS | | MWS | | WS | |
| Locus | Gene | Gene | Score | Domains | S | C | S | C | S | C |
| 10q23.33a | HHEX | IPF1 | 0.571 | Homeobox | 1 | 1 | 3 | 1 | 3 | 1 |
| 21q22.13b | KCNJ6 | KCNJ11 | 0.526 | IRK | 1 | 1 | 1 | 1 | 1 | 1 |
| 22q13.31d | PPARA | PPARG | 0.804 | Hormone_recep\|zf-C4 | 0 | 0 | 0 | 0 | 0 | 0 |
| 12p13.31c | SLC2A3 | SLC2A4 | 0.632 | Sugar_tr | 0 | 0 | 1 | 1 | 1 | 1 |
| 10q24.32b | PITX3 | PAX4 | 0.574 | Homeobox | 0 | 0 | 0 | 0 | 0 | 0 |
| 5q33.2b | HAND1 | PTF1A | 0.532 | HLH | 0 | 0 | 0 | 0 | 0 | 0 |
| 12p12.31c | SLC2A14 | SLC2A4 | 0.615 | Sugar_tr | 0 | 0 | 0 | 0 | 0 | 0 |
| 10q24.32a | KCNIP2 | GPD2 | 0.533 | efhand | 0 | 0 | 0 | 0 | 0 | 0 |
| 15q21.1a | DUOX1 | GPD2 | 0.459 | efhand | 0 | 0 | 0 | 0 | 0 | 0 |
| 5q31.1d | TCF7 | TCF7L2 | 0.998 | CTNNB1_binding\|HMG_box | 0 | 0 | 0 | 0 | 0 | 0 |
| 6p21.31c | PPARD | PPARG | 0.808 | Hormone_recep\|zf-C4 | 0 | 0 | 0 | 0 | 0 | 0 |
| 5q31.1f | NEUROG1 | NEUROD1 | 0.733 | HLH | 0 | 0 | 0 | 0 | 1 | 1 |
| 1p34.2a | SLC2A1 | SLC2A4 | 0.710 | Sugar_tr | 0 | 0 | 0 | 0 | 1 | 1 |
| 20p11.21a | VSX1 | PAX4 | 0.633 | Homeobox | 0 | 0 | 0 | 0 | 0 | 0 |
| 11q24.3b | BARX2 | IPF1 | 0.620 | Homeobox | 0 | 0 | 0 | 0 | 3 | 1 |
| 9q31.1a | NR4A3 | HNF4A | 0.619 | Hormone_recep\|zf-C4 | 0 | 0 | 0 | 0 | 0 | 0 |

| | Adjacent | | | | | | 1Mbp | | | | | |
|---|---|---|---|---|---|---|---|---|---|---|---|---|
| | MHS | | MWS | | WS | | MHS | | MWS | | WS | |
| Locus | S | C | S | C | S | C | S | C | S | C | S | C |
| 10q23.33a | 1 | 1 | 3 | 1 | 3 | 1 | 1 | 1 | 3 | 1 | 3 | 1 |
| 21q22.13b | 1 | 1 | 1 | 1 | 2 | 2 | 1 | 1 | 1 | 1 | 2 | 2 |
| 22q13.31d | 0 | 0 | 0 | 0 | 3 | 1 | 0 | 0 | 2 | 1 | 13 | 1 |
| 12p13.31c | 0 | 0 | 1 | 1 | 1 | 1 | 0 | 0 | 1 | 1 | 1 | 1 |
| 10q24.32b | 0 | 0 | 2 | 1 | 2 | 1 | 0 | 0 | 2 | 1 | 3 | 2 |
| 5q33.2b | 0 | 0 | 1 | 1 | 3 | 1 | 0 | 0 | 1 | 1 | 3 | 1 |
| 12p12.31c | 0 | 0 | 0 | 0 | 0 | 0 | 0 | 0 | 1 | 1 | 1 | 1 |
| 10q24.32a | 0 | 0 | 0 | 0 | 0 | 0 | 0 | 0 | 1 | 1 | 2 | 2 |
| 15q21.1a | 0 | 0 | 0 | 0 | 0 | 0 | 0 | 0 | 1 | 1 | 1 | 1 |
| 5q31.1d | 0 | 0 | 0 | 0 | 1 | 1 | 0 | 0 | 0 | 0 | 0 | 0 |
| 6p21.31c | 0 | 0 | 0 | 0 | 0 | 0 | 0 | 0 | 0 | 0 | 7 | 2 |
| 5q31.1f | 0 | 0 | 0 | 0 | 1 | 1 | 0 | 0 | 0 | 0 | 1 | 1 |
| 1p34.2a | 0 | 0 | 0 | 0 | 1 | 1 | 0 | 0 | 0 | 0 | 1 | 1 |
| 20p11.21a | 0 | 0 | 0 | 0 | 0 | 0 | 0 | 0 | 0 | 0 | 1 | 1 |
| 11q24.3b | 0 | 0 | 0 | 0 | 3 | 1 | 0 | 0 | 0 | 0 | 3 | 1 |
| 9q31.1a | 0 | 0 | 0 | 0 | 1 | 1 | 0 | 0 | 0 | 0 | 1 | 1 |

S - number of SNPs
C - number of clusters formed by SNPs
Genes in bold are those with SNPs within gene boundaries

### Discussion of Example 2

### Effect of SNP Mapping

Most mutations for Mendelian diseases have been found in the ORF or splice sites resulting in a loss of function, or more rarely, a gain of function. The preponderance of Mendelian mutations in ORFs could be the result of a selection effect as the ORF is the first region sequenced. Alternatively, these observations could be real and Mendelian diseases may be largely confined to coding sequence. In contrast the search for susceptibility alleles for complex diseases using traditional techniques that focus on sequencing of the ORF was been largely unproductive. The results from the first Genome Wide Association (some of 1 which are biased to ORFs) indicating that susceptibility alleles for complex disease may instead be associated with introns and intergenic regions. One thing that was immediately apparent was that many of the predictions made by the present invention were for the 1 Mbp BY and adjacent NN mappings. For some phenotypes, very few predictions were returned for the nearest mapping. There are two possibilities for this result: the information from long range effects and bystander genes are ignored in the nearest mapping or the inclusion of more genes simply increases the chance of predictions. For instance, the top pathways predicted by CPS for the CAD phenotype did not have a consistent statistical significance across the mappings (Table 17). It is unclear whether the 1 Mbp BY mapping approach is detecting the distal regulatory control effects on genes or whether more common genes are overwhelming the normalization process.

### Similarity Between Phenotypes

Multiple biological processes were implicated by candidates predicted to be associated with the phenotypes: transcriptional regulation, cell-cell adhesion and cell extracellular matrix (ECM) interactions, cytoskeletal remodeling, membrane transduction of signals: both through Tyrosine kinase receptors, and G-coupled receptors with concommitant generation of intracellular second messengers, RNA and epigenetic processes, membrane transport through ion and solute channels, as well as metabolism, the immune response and protein folding.

TABLE 17
Pathways predicted for CD from the weakly significant set

| | Known | | | | | | | | | Ab initio | | | | | | | | |
|---|---|---|---|---|---|---|---|---|---|---|---|---|---|---|---|---|---|---|
| | Nearest | | | Adjacent | | | 1Mbp | | | Nearest | | | Adjacent | | | 1Mbp | | |
| Pathway | n | r | p | n | r | p | n | r | p | n | r | p | n | r | p | n | r | p |
| Cytokine-cytokine | 13 | 1 | 0.041 | 20 | 1 | 0.702 | 37 | 1 | 0.047 | 12 | 2 | 0.041 | 19 | 3 | 0.702 | 36 | 4 | 0.047 |
| receptor interaction | | | | | | | | | | | | | | | | | | |
| Jak-STAT signaling | 9 | 2 | 0.061 | 18 | 2 | 0.031 | 29 | 2 | 1.000 | 8 | 3 | 0.061 | 17 | 4 | 0.031 | 28 | 6 | 1.000 |
| pathway | | | | | | | | | | | | | | | | | | |
| Role of ERBB2 in Signal | 4 | 3 | 0.020 | 4 | 6 | 0.196 | 4 | 10 | 0.786 | 3 | 8 | 0.020 | 3 | 15 | 0.196 | 3 | 27 | 0.786 |
| Transduction and | | | | | | | | | | | | | | | | | | |
| Oncology | | | | | | | | | | | | | | | | | | |
| Regulation of | 3 | 4 | 0.080 | 5 | 5 | 0.025 | 9 | 5 | 0.009 | 2 | 9 | 0.080 | 4 | 14 | 0.025 | 8 | 22 | 0.009 |
| hematopoiesis by | | | | | | | | | | | | | | | | | | |
| cytokines | | | | | | | | | | | | | | | | | | |
| IL 6 signaling pathway | 3 | 4 | 0.108 | 3 | 7 | 0.654 | 4 | 10 | 0.783 | 2 | 9 | 0.108 | 2 | 16 | 0.654 | 3 | 27 | 0.783 |
| Erythrocyte | 2 | 5 | 0.305 | 4 | 6 | 0.052 | 8 | 6 | 0.006 | — | — | — | 3 | 15 | 0.052 | 7 | 23 | 0.006 |
| Differentiation Pathway | | | | | | | | | | | | | | | | | | |
| Neuroactive ligand- | — | — | — | — | — | — | — | — | — | 13 | 1 | — | 32 | 1 | 0.000 | 41 | 1 | 0.448 |
| receptor interaction | | | | | | | | | | | | | | | | | | |
| Calcium signaling | — | — | — | — | — | — | — | — | — | 7 | 4 | 0.217 | 20 | 2 | 0.019 | 37 | 3 | 0.314 |
| pathway | | | | | | | | | | | | | | | | | | |
| ECM-receptor interaction | — | — | — | — | — | — | — | — | — | 7 | 4 | 0.009 | 9 | 9 | 0.193 | 17 | 13 | 0.891 |
| Adipocytokine signaling | — | — | — | — | — | — | — | — | — | 6 | 5 | 0.011 | 8 | 10 | 0.152 | 17 | 13 | 0.282 |
| pathway | | | | | | | | | | | | | | | | | | |
| Cell Communication | — | — | — | — | — | — | — | — | — | 3 | 8 | 1.000 | 5 | 13 | 0.167 | 11 | 19 | 0.000 |
| Antigen processing and | — | — | — | — | — | — | — | — | — | — | — | — | — | — | — | 6 | 24 | 0.002 |
| presentation | | | | | | | | | | | | | | | | | | |
| The Role of Eosinophils | — | — | — | — | — | — | — | — | — | — | — | — | 3 | 15 | 0.024 | 5 | 25 | 0.017 |
| in the Chemokine | | | | | | | | | | | | | | | | | | |
| Network of Allergy | | | | | | | | | | | | | | | | | | |
| Metabolism of | — | — | — | — | — | — | — | — | — | — | — | — | — | — | — | 6 | 24 | 0.021 |
| xenobiotics by | | | | | | | | | | | | | | | | | | |
| cytochrome P450 | | | | | | | | | | | | | | | | | | |
| Histidine metabolism | — | — | — | — | — | — | — | — | — | — | — | — | — | — | — | 2 | 28 | 0.023 |
| Proteolysis and Signaling | — | — | — | — | — | — | — | — | — | — | — | — | — | — | — | 4 | 26 | 0.030 |
| Pathway of Notch | | | | | | | | | | | | | | | | | | |
| Aminoacyl-tRNA | — | — | — | — | — | — | — | — | — | — | — | — | 6 | 12 | 0.056 | 13 | 17 | 0.036 |
| biosynthesis | | | | | | | | | | | | | | | | | | |
| Natural killer cell | — | — | — | — | — | — | — | — | — | 5 | 6 | 0.259 | 9 | 9 | 0.857 | 16 | 14 | 0.042 |
| mediated cytotoxicity | | | | | | | | | | | | | | | | | | |
| Tyrosine metabolism | — | — | — | — | — | — | — | — | — | — | — | — | 2 | 16 | 0.433 | 5 | 25 | 0.042 |
| Selective expression of | — | — | — | — | — | — | — | — | — | 3 | 8 | 0.033 | 5 | 13 | 0.027 | 9 | 21 | 0.043 |
| chemokine receptors | | | | | | | | | | | | | | | | | | |
| during T-cell polarization | | | | | | | | | | | | | | | | | | |
| Phenylalanine, tyrosine | — | — | — | — | — | — | — | — | — | — | — | — | 4 | 14 | 0.003 | 4 | 26 | 0.077 |
| and tryptophan | | | | | | | | | | | | | | | | | | |
| biosynthesis | | | | | | | | | | | | | | | | | | |
| T cell receptor signaling | — | — | — | — | — | — | — | — | — | 3 | 8 | 0.737 | 12 | 6 | 0.034 | 21 | 9 | 0.346 |
| pathway | | | | | | | | | | | | | | | | | | |
| Actions of Nitric Oxide in | — | — | — | — | — | — | — | — | — | 2 | 9 | 0.080 | 4 | 14 | 0.038 | 7 | 23 | 0.064 |
| the Heart | | | | | | | | | | | | | | | | | | |
| IL 3 signaling pathway | — | — | — | — | — | — | — | — | — | — | — | — | 3 | 15 | 0.041 | 4 | 26 | 0.294 |
| Dendritic cells in | — | — | — | — | — | — | — | — | — | 2 | 9 | 0.099 | 4 | 14 | 0.046 | 5 | 25 | 0.568 |
| regulating TH1 and TH2 | | | | | | | | | | | | | | | | | | |
| Development | | | | | | | | | | | | | | | | | | |
| Basal cell carcinoma | — | — | — | — | — | — | — | — | — | 5 | 6 | 0.016 | 7 | 11 | 0.102 | 12 | 18 | 0.609 |
| Repression of Pain | — | — | — | — | — | — | — | — | — | 2 | 9 | 0.017 | 2 | 16 | 0.137 | 3 | 27 | 0.389 |
| Sensation by the | | | | | | | | | | | | | | | | | | |
| Transcriptional Regulator | | | | | | | | | | | | | | | | | | |
| DREAM | | | | | | | | | | | | | | | | | | |
| Hedgehog signaling | — | — | — | — | — | — | — | — | — | 5 | 6 | 0.020 | 8 | 10 | 0.057 | 10 | 20 | 1.000 |
| pathway | | | | | | | | | | | | | | | | | | |
| Th1/Th2 Differentiation | — | — | — | — | — | — | — | — | — | 3 | 8 | 0.020 | 3 | 15 | 0.177 | 6 | 24 | 0.253 |
| Regulation of | — | — | — | — | — | — | — | — | — | 2 | 9 | 0.022 | 2 | 16 | 0.112 | 3 | 27 | 0.189 |
| Spermatogenesis by | | | | | | | | | | | | | | | | | | |
| CREM | | | | | | | | | | | | | | | | | | |
| Neurodegenerative | — | — | — | — | — | — | — | — | — | 4 | 7 | 0.023 | 5 | 13 | 0.197 | 10 | 20 | 0.311 |
| Diseases | | | | | | | | | | | | | | | | | | |
| Deregulation of CDK5 in | — | — | — | — | — | — | — | — | — | 2 | 9 | 0.028 | 2 | 16 | 0.163 | 2 | 28 | 1.000 |
| Alzheimers Disease | | | | | | | | | | | | | | | | | | |
| Cyclins and Cell Cycle | — | — | — | — | — | — | — | — | — | 3 | 8 | 0.033 | 3 | 15 | 0.416 | 5 | 25 | 1.000 |
| Regulation | | | | | | | | | | | | | | | | | | |
| Regulation of p27 | — | — | — | — | — | — | — | — | — | 2 | 9 | 0.048 | 2 | 16 | 0.274 | 5 | 25 | 0.165 |
| Phosphorylation during | | | | | | | | | | | | | | | | | | |
| Cell Cycle Progression | | | | | | | | | | | | | | | | | | |

Involvement of multiple transcription factors was implicated in six phenotypes by CMP ab initio input mode. At the transcriptional level CAD stood out as the only phenotype where no transcription factors were predicted to be associated with the disease. Families of transcription factors associated with HT were markedly different to the other four phenotypes. Similar families of transcription factors were common to three phenotypes-RA, T1D, CD, and interestingly, BD also showed interesting similarities. RA, T1D and CD are all well known as autoimmune phenotypes. Interestingly, a member of one of these families, the ETS transcription factors, has previously been associated with autoimmunity. Thus at the transcriptional level, BD bears some resemblance to autoimmune diseases. A link between bipolar and autoimmune thyroiditis has been suggested, which is interesting in the light of prediction of the thyroid hormone3 binding nuclear hormone receptor THRB for BD. Not many families of transcription factors were predicted for T2D but multiple hormone receptors were associated with both the diabetic phenotypes, T2D and T1D. Nuclear hormone receptors integrate complex metabolic homeostasis and thus metabolic dysfunction is implicated in both diabetic phenotypes. Defects in the nuclear hormone receptor PPARG can lead to type 2 insulin resistant diabetes. The nuclear receptor PPARG/RXRA heterodimer regulates glucose and lipid homeostasis and is the target for the antidiabetic drugs G1262570 and the thiazolidinediones (TZDs) but have not previously been associated with T1D.

Protein folding and generation was implicated in four phenotypes but the genes were largely phenotype-specific. Heat shock proteins were predicted in CAD and RA. Genes involved in glycosylation were predicted in four phenotypes. For CAD and T2D, genes involved with O-glycosylation were predicted, whereas two genes involved in N-glycosylation were predicted in Crohn's. Two genes involved in GAG synthesis were implicated in BD by CMP ab initio. These were independently implicated by CPS ab initio for the BP phenotype along with a further three genes involved in heparan sulfate biosynthesis.

At the metabolic level, mitochondrial catabolism of amino or fatty acids is implicated in three phenotypes: CAD, T2D and BD. This is interesting in the light of the involvement of metabolic syndrome in these diseases. Metabolic syndrome is characterized by abdominal obesity, high triglycerides, low levels of high density lipoprotein cholesterol (HDLC), high blood pressure, and elevated fasting glucose levels. It is estimated that around 75% of patients with T2D and 50% of patients with CAD have metabolic syndrome and as many as 70% of patients with BP. Mitochondrial defects have previously been implicated in metabolic syndrome with a decrease of mitochondria in skeletal muscle suggested as an aetiology. Defects in metabolism may also contribute. The IVD and ACAD8 genes coding for proteins that catabolise the branched amino acids leucine and valine, respectively, were common to the CAD, BP and T2D phenotypes. In addition, fatty acid catabolism was implicated in T2D by ACAD9. Hypoglycemia is a component of the ACAD9 deficiency phenotype (MIM: 611103). The implication of Lys and Trp catabolism in BP by GCDH is significant because the mood-affecting neurotransmitter serotonin is derived from Trp. Metabolic dysfunction is implicated in both diabetic phenotypes by the involvement of nuclear hormone receptors, which integrate complex metabolic homeostasis.

Epigenetic processes were implicated in four of the phenotypes. Chromatin remodeling was implicated via helicase genes predicted in the vascular phenotypes CAD and HT, as well as in RA. Multiple potential epigenetic mechanisms were suggested in BP by genes disrupting the binding of chromatin to histones, or mediating binding of heterochromatin near centromeres. The PADI genes can irreversibly citrinillate arginine residues in histones, and two genes which methylate lysine residues, MLL2 and TBRG1 were implicated in BP. Multiple histone genes were implicated in T1D.

Control of cell division was implicated in three phenotypes: RA, CAD and CD. Premature atherosclerosis has been observed during the course of different systemic inflammatory diseases such as RA and sytemic lupus erythematosus.

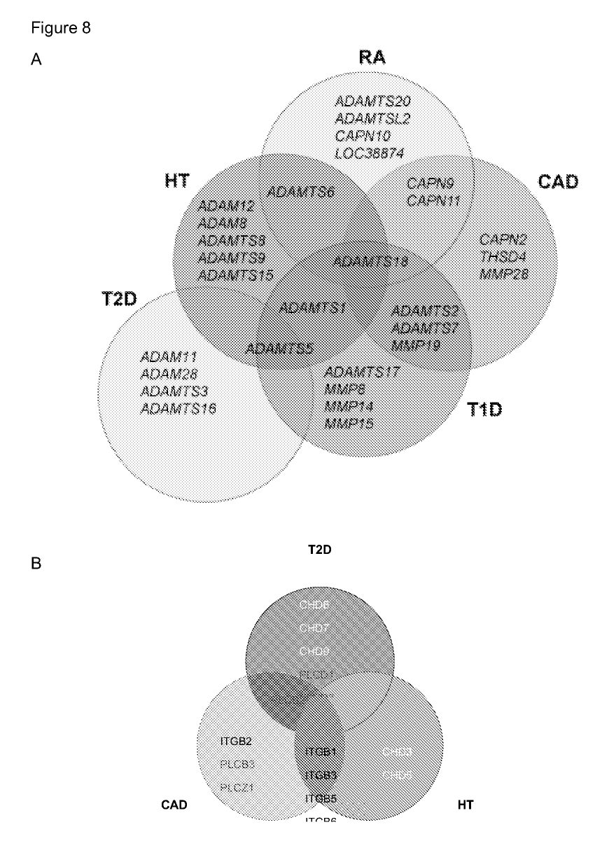

Interactions between integrins and 1 the extracellular matrix was implicated in RA, CAD and HT by integrin β chains and laminins. The involvement of thrombospondins which support the role of laminins, but do not act in dependently, was additionally implicated in HT and CAD. Maintenance of the actin cytoskeleton featured in CAD, Crohn's disease and RA. Proteins with FERM domains were predicted for all three phenotypes. In addition proteins involved with actin treadmilling were predicted for RA, while genes involved in stabilization of F-actin were implicated for CAD and transmembrane adaptor proteins mediating interaction with extracellular collagen were implicated in CD. Cell-cell adhesion was also a theme. The prediction of the tight junction protein PGM5 and the related PGM1 is interesting in the light of the proposed role of epithelial tight junctions in intestinal inflammation (Schulzke, 2009). With regard to cell-cell adhesion and cell-ECM adhesion there were interesting similarities between CAD and RA. Some overlap between genes underlying the phenotypes: zinc metalloproteases, in particular those with thrombospondin domains (ADAMTS) were implicated in all three phenotypes. However, with the exception of ADAMTS5 which was implicated in both T2D and HT, the particular genes involved were phenotype-specific (FIG. 8). ADAMs, which are homologous but lack the thrombospondin domain were implicated in HT and T2D but matrix metalloproteases were highlighted instead in CAD. Integrins were implicated in the HT and CAD phenotypes. Phospholipases and actin-binding cytoskeletal proteins featured in T2D and CAD. Ephrin receptors are implicated in both diabetes phenotypes and also in Crohn's disease: ephrin A recetors in diabetes-EPHA4 and EPHA5 in T2D and EPHA5, 7 & 10 in T1D, ephrin A4 and ephrin B5 are implicated in CD. Bi-directional signalling co-ordinates cell interactions through Ephrin receptors on one cell and Ephrin ligands on the other cell. Potential ephrin receptor interactors which are also predicted candidates are the NOTCH proteins (T1D), the P13 kinases (T1D) and ADAMTS proteases (T1D).

Proteolytic cleavage not only terminates the adhesive Eph-ephrin interaction and causes downregulation of the proteins, but it can also generate Eph/ephrin fragments with new activities (Pasquale, 2008). There is crosstalk between EPH and WNT signalling pathways in the intestinal epithelium and candidates from both pathways are implicated. There is also cross-talk between EPH and integrin pathways. Integrins, which mediate interactions with the ECM, are implicated in the CAD (Integrins B1-5), HT (Integrins B1,3,5-6), RA (Integrins B1,3). Matrix metalloproteases which remodel the ECM are implicated in CAD (MMP15 & 19) and HT (MMP 2, 15, 21, 24) and T1D (MMP8, 14, 19-20, 27, 28). E-cadherin-dependent intercellular adhesion can also regulate Eph receptor expression, cell-surface localization, and ephrin-dependent activation. The regulation is reciprocal, and EphB signaling drives E-cadherin to the cell surface thus promoting the formation of epithelial adherens junctions and enabling EphB/ephrin-B-dependent cell sorting. Cadherins are implicated five phenotypes: CAD (CDH4,7,13,19, DSC3), CD (CDH8,10), RA (CDH4,7,8,9,10,19), T2D (CDH4,5,8,9,10,11). Finally Adherens junctions are implicated in CD, by PGM5.

Secondary messengers were implicated in numerous phenotypes. G-coupled receptors are common to several phenotypes. Metatropic glutamate receptors are implicated in CD, RA and HT (GRM3,5,7,8). Adhesion G-couple receptors are implicated in CAD, T2D and CD (Frizzled).

At the phenotype level, Rheumatoid arthritis (RA) is an inflammatory disease associated with premature atherosclerosis. Predicted genes common to these two phenotypes included heat shock proteins, ATP-dependent chromatin remodelling helicases, multiple proteins involved in cell-cell and cell-ECM interactions including integrin β-chains, laminins, cadherins, actin cytoskeleton-interacting proteins and proteins that remodel these interactions including calpains and ADAMTS zinc metalloproteases. The two diabetic phenotypes had share various signalling proteins including RasGAP proteins, Ephrin receptor tyrosine kinases, and multiple nuclear hormone receptors. Adults with BD-I are at increased risk of CAD and HT123. Abnormal glutaminergic and Ca-activated ion channel control was suggested for the BD and HT phenotypes, as well as tyrosine kinase receptors controlling growth and proliferation, proteins of synaptic vesicles, scavenger receptors. There were fewer common predictions for bipolar and CAD but they included CUB/shear adhesion molecules which may play a role in cell-cell recognition and neuronal membrane signalling, and enzymes of mitochondrial metabolism.

### Known Disease Gene Input Mode Versus Ab Initio Input Mode

Using a known disease set assumes that the disease phenotype is a complete picture of the disease. This is compensated through the ab initio methodology. In the cases of diseases with Mendelian inheritance it would be advisable to try ab initio mode if only a small percentage of cases arise from existing pathways for the discovery of novel implications. CPS ab initio may have implicated novel pathways, but in most of the cases these pathways involved candidate genes predicted from the known pathways. In the case of CMP, known mode predicted few candidates and was dependent on the phenotype. Diseases such as BD and CD did not have many predictions (Table 18 and Table 19).

Most CMP ab initio results are those from the 1 Mbp and adjacent mapping approaches.

The present invention made multiple predictions which were not implicated by the WTCCC study.

Limitations of sole NN Approaches and Appraisal of by Mapping

The present inventors have shown that studies only using a nearest neighbor approach are essentially blind to around one quarter of the genome due to poor annotation that could be associated with a phenotype. Additionally, the search space has been limited by SNP to gene mapping before the evaluation has even begun. As a result, alternate approaches such as the bystander assumptions increase the gene coverage of the genome, but require stricter filtering as much more noise is introduced into the results.

TABLE 18
BD CMP known results

| | | | | | Nearest | | | | | | Adjacent | | | | | | 1Mbp | | | | | |
|---|---|---|---|---|---|---|---|---|---|---|---|---|---|---|---|---|---|---|---|---|---|---|
| | | Known | | Common | MHS | | MWS | | WS | | MHS | | MWS | | WS | | MHS | | MWS | | WS | |
| Locus | Gene | Gene | Score | Domains | S | C | S | C | S | C | S | C | S | C | S | C | S | C | S | C | S | C |
| 14q32.33a | KNS2 | FKBP5 | 0.35 | TPR_1 | 0 | 0 | 0 | 0 | 0 | 0 | 0 | 0 | 0 | 0 | 0 | 0 | 2 | 2 | 3 | 3 | 3 | 3 |
| 16q12.2c | SLC6A2 | SLC6A3 | 0.741 | SNF | 0 | 0 | 0 | 0 | 0 | 0 | 0 | 0 | 0 | 0 | 0 | 0 | 0 | 0 | 3 | 2 | 6 | 2 |
| 20p13b-p13a | ADRA1D | HTR2A | 0.256 | 7tm_1 | 0 | 0 | 0 | 0 | 1 | 1 | 0 | 0 | 0 | 0 | 1 | 1 | 0 | 0 | 2 | 1 | 3 | 2 |
| 20q13.12b | TOMM34 | FKBP5 | 0.546 | TPR_1 | 0 | 0 | 0 | 0 | 0 | 0 | 0 | 0 | 0 | 0 | 0 | 0 | 0 | 0 | 1 | 1 | 1 | 1 |
| 12q21.32a | TMTC3 | FKBP5 | 0.405 | TPR_1 | 0 | 0 | 0 | 0 | 1 | 1 | 0 | 0 | 0 | 0 | 5 | 2 | 0 | 0 | 0 | 0 | 5 | 2 |
| 3p25.3a | SLC6A11 | SLC6A3 | 0.462 | SNF | 0 | 0 | 0 | 0 | 1 | 1 | 0 | 0 | 0 | 0 | 1 | 1 | 0 | 0 | 0 | 0 | 1 | 1 |
| 2p24.1d | TTC32 | FKBP5 | 0.396 | TPR_1 | 0 | 0 | 0 | 0 | 0 | 0 | 0 | 0 | 0 | 0 | 3 | 1 | 0 | 0 | 0 | 0 | 3 | 1 |
| 14q31.3d | TTC8 | FKBP5 | 0.349 | TPR_1 | 0 | 0 | 0 | 0 | 0 | 0 | 0 | 0 | 0 | 0 | 1 | 1 | 0 | 0 | 0 | 0 | 1 | 1 |
| 13q12.11b | IFT88 | FKBP5 | 0.381 | TPR_1 | 0 | 0 | 0 | 0 | 0 | 0 | 0 | 0 | 0 | 0 | 1 | 1 | 0 | 0 | 0 | 0 | 1 | 1 |
| 17q21.32a | CDC27 | FKBP5 | 0.388 | TPR_1 | 0 | 0 | 0 | 0 | 0 | 0 | 0 | 0 | 0 | 0 | 1 | 1 | 0 | 0 | 0 | 0 | 1 | 1 |
| 15q24.1a | BBS4 | FKBP5 | 0.397 | TPR_1 | 0 | 0 | 0 | 0 | 0 | 0 | 0 | 0 | 0 | 0 | 1 | 1 | 0 | 0 | 0 | 0 | 1 | 1 |
| 3q22.1c | NPHP3 | FKBP5 | 0.361 | TPR_1 | 0 | 0 | 0 | 0 | 0 | 0 | 0 | 0 | 0 | 0 | 1 | 1 | 0 | 0 | 0 | 0 | 1 | 1 |
| 10q23.31d | HTR7 | HTR2A | 0.291 | 7tm_1 | 0 | 0 | 0 | 0 | 0 | 0 | 0 | 0 | 0 | 0 | 1 | 1 | 0 | 0 | 0 | 0 | 1 | 1 |
| 3p25.3a | SLC6A1 | SLC6A3 | 0.502 | SNF | 0 | 0 | 0 | 0 | 0 | 0 | 0 | 0 | 0 | 0 | 0 | 0 | 0 | 0 | 0 | 0 | 1 | 1 |
| 19p13.3g | SGTA | FKBP5 | 0.454 | TPR_1 | 0 | 0 | 0 | 0 | 0 | 0 | 0 | 0 | 0 | 0 | 0 | 0 | 0 | 0 | 0 | 0 | 1 | 1 |
| 22q12.1c | TTC28 | FKBP5 | 0.373 | TPR_1 | 0 | 0 | 0 | 0 | 0 | 0 | 0 | 0 | 0 | 0 | 0 | 0 | 0 | 0 | 0 | 0 | 1 | 1 |
| 22q11.23b | CABIN1 | FKBP5 | 0.333 | TPR_1 | 0 | 0 | 0 | 0 | 0 | 0 | 0 | 0 | 0 | 0 | 0 | 0 | 0 | 0 | 0 | 0 | 2 | 1 |
| 5q33.1b | ADRB2 | HTR2A | 0.277 | 7tm_1 | 0 | 0 | 0 | 0 | 0 | 0 | 0 | 0 | 0 | 0 | 0 | 0 | 0 | 0 | 0 | 0 | 8 | 1 |
| 12p11.22a | TMTC1 | FKBP5 | 0.354 | TPR_1 | 0 | 0 | 0 | 0 | 0 | 0 | 0 | 0 | 0 | 0 | 1 | 1 | 0 | 0 | 0 | 0 | 0 | 0 |

S - number of SNPs
C - number of clusters formed by SNPs
Genes in bold are those with SNPs within gene boundaries

TABLE 19
CD CMP known results

| | | | | | Nearest | | | | | | Adjacent | | | | | | 1Mbp | | | | | |
|---|---|---|---|---|---|---|---|---|---|---|---|---|---|---|---|---|---|---|---|---|---|---|
| | | Known | | Common | MHS | | MWS | | WS | | MHS | | MWS | | WS | | MHS | | MWS | | WS | |
| Locus | Gene | Gene | Score | Domains | S | C | S | C | S | C | S | C | S | C | S | C | S | C | S | C | S | C |
| 5q31.1a | RAPGEF6 | DLG5 | 0.336 | PDZ | 0 | 0 | 0 | 0 | 0 | 0 | 0 | 0 | 0 | 0 | 0 | 0 | 0 | 0 | 1 | 1 | 10 | 3 |
| 8q11.22a-q11.22c | SNTG1 | DLG5 | 0.26 | PDZ | 0 | 0 | 0 | 0 | 0 | 0 | 0 | 0 | 0 | 0 | 1 | 1 | 0 | 0 | 0 | 0 | 1 | 1 |
| 1q23.1b | ARHGEF11 | DLG5 | 0.255 | PDZ | 0 | 0 | 0 | 0 | 0 | 0 | 0 | 0 | 0 | 0 | 0 | 0 | 0 | 0 | 0 | 0 | 3 | 1 |
| 1q21.3a | SNX27 | DLG5 | 0.274 | PDZ | 0 | 0 | 0 | 0 | 0 | 0 | 0 | 0 | 0 | 0 | 0 | 0 | 0 | 0 | 0 | 0 | 1 | 1 |
| 19q13.33a | LIN7B | DLG5 | 0.323 | PDZ | 0 | 0 | 0 | 0 | 0 | 0 | 0 | 0 | 0 | 0 | 0 | 0 | 0 | 0 | 0 | 0 | 1 | 1 |
| 9q21.11a | TJP2 | DLG5 | 0.291 | PDZ\|SH3_2 | 0 | 0 | 0 | 0 | 0 | 0 | 0 | 0 | 0 | 0 | 0 | 0 | 0 | 0 | 0 | 0 | 1 | 1 |

S - number of SNPs
C - number of clusters formed by SNPs
Genes in bold are those with SNPs within gene boundaries

Transcription factor binding sites, promoters, enhancers, long range, cis and trans regulatory regions. Dispersed genetic architecture for example long range enhancers and regulators. Taking genes closest to the SNP may ignore a link to a gene further away that may be a more likely candidate.

More generous mappings did not unduly lower the performance of the system.

### Limitations of Annotations

Annotations and analyses are as accurate as underlying databases. Some pathways are actually groups of pathways, so random sampling of genes will yield significant results when these genes are found in the pathway group, but are not part of distinct paths.

Some pathways are actually groups of pathways, so random sampling of genes will yield significant results when these genes are found in the pathway group, but are not part of distinct paths.

In example 1, which used a dataset developed by Turner et al (2003), with more Mendelian diseases, CPS was more informative but on genome wide association data, CMP unexpectedly performed better. The modular domain-based CMP approach is unique. The metric calculated in CMP removes the need to rely on the current annotations of human proteins which are still lacking or on sequence-similarity which is less accurate.

It has been observed that the same pathways are involved in complex diseases as Mendelian diseases with similar phenotypes. In the case of Mendelian disease, a single rare mutation critical to the function of one gene can grossly disturb the function of the pathway or protein complex. Similar mutations in other genes in a pathway can lead to largely similar but often distinguishable Mendelian diseases. In a complex disease, multiple SNPs common in the population may contribute to less effective functioning of the pathway which may also be impaired or stressed by environmental factors. Mutations in the regulatory regions alter expression levels of proteins which may affect the dynamic range of signaling pathways. For most complex diseases a combination of one or more susceptibility alleles as well as environmental stimuli may be required to alter the dynamic range sufficiently to invoke the disease state.

### Drug Discovery Pipeline

Target identification and validation is a crucial first step in developing a drug against a given disease. Only 20-30 new chemical entities are approved as drugs in the US each year and only a quarter of these will act on targets not already hit by an existing drug. There is a real need to identify new targets to treat human disease. The present invention can be expanded into an informatics driven drug-discovery pipeline, which will utilise data from the human genome and disease databases to identify druggable-targets for all diseases.

A target is only of value if it can be related to a disease. This process can take many years as target validation is often a multi-step process involving studies in epidemiology, disease physiology and results from animal models. However, in Mendelian disorders, the inheritance of a mutation in a single gene can be linked directly to a phenotype. There are over 5000 phenotypes with a Mendelian pattern of inheritance, and the gene responsible has been identified in approximately 1200 of these (OMIM). The present invention can be used to identify the disease gene for a further 1500 disease loci for which the disease gene remains undetermined

In the past, pharmaceutical companies have not studied these diseases, either because the affected protein is not amenable to drug intervention, or more likely, the number of people affected is small and, therefore, drug discovery is not economically viable. Patients with uncommon disorders are often neglected and only receive medications that have come from treatments developed for other more common disorders. However, these neglected diseases may hold the key to therapies that could have multiple uses. A single gene in Mendelian disease may provide insight into complex diseases where the same gene accounts for part of the phenotype. For example, statin therapy was specifically developed to patients with a genomic predisposition to high levels of blood cholesterol, but is equally effective for patients with the same condition but from multiple causes.

### Mapping Diseases to the Human Genome

All disease genes and intervals will be extracted from OMIMs morbidmap (downloadable file), OMIM webpages and the literature. The invention can be used to make predictions for possible disease intervals with unknown disease genes. The minimal requirement for prediction is typically one disease gene or two characterized disease intervals with the same or similar phenotypes.

Benchmarking shows that the invention is already better than published candidate gene prediction systems. Currently our CMP method applies Pfam HMMs to annotate candidate proteins, however, Pfam only has coverage for about 65% of the proteins in the human genome. Domain coverage can be extended by using a combined method of domain prediction and threading. The scooby-domain algorithm (George R A, Lin K and Hering a J (2005) Scooby-domain: prediction of globular domains in protein sequence. Nucleic Acids Res 33, W160-W163) and DOMAINATION methodology (George R A, Hering a J. (2002) Protein domain identification and improved sequence similarity searching using PSI-BLAST. Proteins. 48,672-81) can be applied to identify putative domains in proteins without Pfam annotation. These domains will then be threaded against a database of domains with known structure and function. Each disease will have associated pathways extracted from Biocarta and KEGG as well as interaction data from OPHID. Complete domain (module) annotation, pathway data and interaction data will be used by CMP and CPS to identify disease genes.

### Efficient Target and Drug Identification

Most successful drugs achieve their activity by competing for a binding site on a protein with an endogenous small molecule. For a drug to be effective, it must bind to its molecular target with a reasonable degree of potency as well as having an increased likelihood of oral bioavailability (Lipinski's rule-of-five). These strict physiochemical requirements will limit the type of targets that are druggable. A protein target should favour interactions with drug-like compounds. Proteins lacking these features are unlikely to be amenable to therapeutics. The chance of identifying a good target will be increased by focusing on proteins that are known to bind with successfully commercialized drugs. Information on proteins known to be druggable is freely available from DrugBank (Wishart et al. 2006). Each module in a protein/gene sequence can be assigned a profile that associates drug-binding characteristics. Likely drug-targets in the human genome can be identified through homology searches with the assigned modules in DrugBank. Proteins do not work in isolation: while the disease gene may not be readily druggable, there might be more suitable targets found in its corresponding pathways or interaction partners. For example, inherited mutations in APC, a component of the Wnt pathway, can lead to colon cancer. APC is difficult to target, but compounds that block downstream interactions in this pathway are able to suppress growth of tumors arising from the APC mutations. By using interaction and pathway data from the BioCarta, KEGG and OPHID databases we can identify disease pathways and potential targets.

Potential drugs for both monogenic and complex diseases can be sourced from already available medications, most of which are now off patent, that can be repositioned to new uses. Detailed information related to dosing, in vivo pharmacokinetics and toxicity are already available for these drugs. Our pipeline will identify whether a current drug will be suitable and can potentially lead to immediate phase III clinical trials that can be performed sooner and more economically.

### Target Identification Through Opposing Phenotypes

Most drugs antagonize the gene product producing phenotypes that are analogous to loss-of-function mutations in human disease. Therefore, monogenic human disorders provide an ideal source of drug targets. Because mutations alter the level of activity of gene products, they can be thought of as surrogates for perfectly targeted drugs, to agonize or antagonize the gene product. An example is sulphonylureas. These drugs function antagonistically through the receptor SUR1 complex. Loss-of-function mutations in the genes that encode components of this complex cause the rare genomic disorder persistent hyperinsulinaemic hypoglycaemia of infancy (PHHI). The phenotype of PHHI is directly mimicked by the action of the sulphonylureas. Mutations that cause monogenic disorders have been identified in the genes that encode 12 out of the 43 protein targets of the top-selling 100 drugs in 2003.

Two methods for candidate disease gene prediction have been developed. CPS hypothesizes that novel disease genes reside in the same pathways as those of known disease genes and CMP assumes that novel disease-causing genes that produce the same phenotype as known disease genes are likely to have similar functions. The genes in the genomic interval of interest are then tested for relationships to known disease genes or genes in other disease intervals. Both CPS and CMP can effectively recover known disease genes from a broad array of diseases.

Many previous candidate gene prediction methods have relied on functional annotation, such as GO terms, which can be general or absent. Only 25% of human proteins have manually annotated GO terms. Many more human proteins have predicted annotations, but 35% have no annotation at all. Furthermore, these systems will be biased to well studied and well annotated diseases and may not be useful in the analysis of uncharacterized diseases.

The methods of the present invention are based directly on biological data, and differ from older candidate gene prediction techniques which use blanket systems based on descriptive keywords to cover all aspects of disease. Such methods include POCUS, G2D and SUSPECTS. New systems biology approaches to candidate gene predictions, which are based directly on biological data, mine PPI and pathway databases. Those described by Franke et al. 2006 as well as our own CPS fall into this category. Our CMP method is quite different to any other method previously described, in that it tries to associate particular protein modules with specific diseases. Not only does this technique represent a more powerful way of finding homologs than BLAST searches but it also has the potential to find otherwise unrelated proteins that engage in homophilic interactions (for example through EGF domains) or share a common functional unit but are otherwise unrelated, for example the protein kinase domains found in thyroid carcinoma.

Comparison with other methods is difficult as benchmark datasets are different and some methods merely rank candidates without applying a cut-off. In an attempt to fairly assess our methods compared to others in example 1, we have used the disease set as applied in the analysis of POCUS. Turner et al previously compared other methods against POCUS by calculating and comparing enrichment ratios: van Driel et al. studied eight diseases and reduced an average 163 genes to 22, producing a seven-fold enrichment. Freudenberg and Propping found two-thirds of disease genes in the top 15% of candidates, giving a seven-fold enrichment. Generally, these keyword methods have been shown to provide a seven to 10-fold enrichment. The updated G2D method is the most successful of these methods, correctly identifying disease genes for 47% of diseases within their ranked top eight predictions, which is below our performance. Using known disease genes as input, we correctly predicted disease genes for 69% of diseases with an average success rate of one in seven (14%) gene predictions and a 13-fold enrichment.

There are only two other methods, POCUS and PRIORITISER, that attempt the more ambitious task of ab initio predictions in the absence of known disease genes. While POCUS makes very few predictions, for the eight diseases that it does make predictions (28%), the quality of prediction is high with a one in four success rate and 23-fold enrichment. The PRIORITISER method by Franke et al. 2006 correctly identified disease genes for 64% of diseases with a success rate of one in eight predictions and a 2.8-fold enrichment. Our combined methods make correct predictions for all diseases with a 2.2-fold enrichment. Another consideration when comparing these results is the range of pseudo-interval sizes used in the benchmark. POCUS used pseudo-intervals based on keyword densities and sizes ranged from 2 to 19 Mb, which are small and more typical of monogenic diseases. Franke et al. 2006 used intervals of 50, 100 and 150-genes, but only included those genes that had predicted interactions. Our benchmark pseudo-intervals range from 50 genes (from 1 Mb) to 150 genes (up to 51 Mb). The larger interval sizes are realistic for complex diseases and include all genes.

Our side-by-side use of two prediction systems in example 1 based directly on independent biological data shows the value of this approach. Several prediction systems were benchmarked against each other using obesity and type 2 diabetes phenotypes. A meta-analysis was then used to choose the best candidates based on consensus. The complementarity of data predicted by our two systems (FIG. 5) show that a consensus method is not always appropriate. Had we used this approach far fewer disease genes would have been found. Clearly the independence of data sources needs to be considered before applying consensus approaches. On the other hand, the type of relationships flagged by CMP is clearly related to pathway data. Pathways may expand by gene duplication and subsequent specialization of the daughters, possibly in association with discrete tissue expression. Similarly, protein complexes consisting of homo-oligomers may differentiate by duplication and specialization of genes encoding similar subunits. If pathway and interaction data were comprehensive then the alternative predictions provided by CMP may not be necessary, but clearly this is not yet the case.

Given that several systems biology approaches have now been published, it is worthwhile examining the caveats associated with these methodologies. CPS with PPI data alone found the majority of disease genes in the benchmark tests. But, some of the interaction data is likely to be dubious, because high-throughput experiments such as yeast two-hybrid and TAP systems will associate proteins that would otherwise never be present in the same cell or subcellular compartment. Furthermore, the various PPIs curated from computational searches of the literature have limited overlap with each other, which may be indicative of a high false positive rate. While there is strong evidence to suggest that PPIs are conserved through evolution, errors in the source data will perpetuate through the databases. These caveats make predicted interactions, such as the Bayesian approach applied by Franke et al., inaccurate. As more evidence for PPIs are collected, the performance of CPS and other similar methods will improve. The results using PPI data alone are already very encouraging: the full OPHID dataset enriches the candidate list by 50-fold, far better than any other reported method.

Finally, although some of the predicted disease genes are not currently known to be involved in the disease, which are counted as false positives in this invention, it is possible that they may be uncharacterized disease-genes. Our methods are also available to identify potential disease genes in user-specified intervals.

A new era of genomics and bioinformatics has permitted a genome-scale perspective of disease and is enabling new technologies to identify disease-causing systems. The present invention should accelerate the disease gene discovery process by gathering and sifting through all knowledge of each candidate gene including its homologues and interaction partners. In addition, it should significantly reduce the cost of expensive experimental studies. Identification of the disease gene enables targeted research on how mutations in the gene contribute to disease and provides specific leads towards cures. The results using the present invention are better than other reported methods for disease gene prediction. Previous methods have relied on functional annotation alone, such as GO terms, which can be general or absent. CPS and CMP utilise information from protein sequence and interaction databases, enabling accurate disease gene identification. In the multiple interval input mode, the present invention does not require a priori knowledge of the disease or disease genes. The present invention should, therefore, be a powerful tool in candidate disease gene prediction for poorly characterised diseases.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A system for profiling a genomic sequence comprising:
(a.) assigning modules to a genome, wherein each module has a defined sequence characteristic and the genome is divided into modules;
(b.) assigning a value or weight to a module for a given profile, wherein the presence of one or more modules in a genomic sequence contributes to the profile of the genomic sequence relative to its value or weight;
(c.) analysing a genomic sequence to identify modules present; and
(d.) assigning a profile to the genomic sequence based on the presence of the modules and their respective value or weight.

2. The system according to claim 1 wherein the genomic sequence is an amino acid sequence of a protein and each module is a universal re-occurring unit found in protein sequences.

3. The system according to claim 1 wherein the genome forms the encoding region and the encoding region is divided into different modules.

4. The system according to claim 1 wherein the profile is selected from the group consisting of a gene or loci associated with a phenotype, disease, drug-binding characteristic, trait associated to pharmacogenomics, associated interacting genes, association with a phenotype, associated or interacting modules, and associated biochemical pathways, and associated modules within biochemical pathways or interacting models with profiles with characteristics described here.

5. The system according to claim 4 wherein the phenotype is a disease or a quantitative trait locus (QTL).

6. The system according to claim 4 wherein the profile is an association with a disease.

7. The system according to claim 4 wherein the profile is a drug-binding characteristic.

8. The system according to claim 1 wherein a given value or weight of a module assigned to a profile is obtained by identifying modules associated with a given phenotype (directly or indirectly through pathways or complexes) and assigning a score based on the similarity of a module to modules associated with a specific phenotype.

9. The system according to claim 1 wherein a given value or weight of a module assigned to a profile is obtained by identifying enrichment of those modules in loci (genomic regions) known to be associated with the phenotype.

10. The system according to claim 1 wherein a module is assigned a value or weight according to its presence in sequences associated with the profile.

11. A system for profiling an amino acid sequence to identify an associated profile, the system comprising:
(a.) assigning modules to the protein coding region of a genome to divide the genome into modules, wherein each module has a defined amino acid characteristic;
(b.) assigning a value or weight to a module for a given profile, wherein the presence of one or more modules in an amino acid sequence contributes to the profile of the sequence relatively to its value or weight;
(c.) analysing an amino acid sequence to identify modules present; and
(d.) assigning a profile to the amino acid sequence based on the presence of the modules and their respective value or weight.

12. The system according to claim 11 wherein the profile is selected from the group consisting of a gene or loci associated with a phenotype, disease, drug-binding characteristic, trait associated to pharmacogenomics, associated interacting genes, association with a phenotype, associated or interacting modules, and associated biochemical pathways, and associated modules within biochemical pathways or interacting models with profiles with characteristics described here.

13. The system according to claim 12 wherein the phenotype is a disease or a quantitative trait locus (QTL).

14. The system according to claim 12 wherein the profile is an association with a disease.

15. The system according to claim 12 wherein the profile is a drug-binding characteristic.

16. The system according to claim 11 wherein a given value or weight of a module assigned to a profile is obtained by identifying modules associated with a given phenotype (directly or indirectly through pathways or complexes) and assigning a score based on the similarity of a module to modules associated with a specific phenotype.

17. The system according to claim 11 wherein a given value or weight of a module assigned to a profile is obtained by identifying enrichment of those modules in loci (genomic regions) known to be associated with the phenotype.

18. The system according to claim 11 wherein a module is assigned a value or weight according to its presence in sequences associated with the profile.

19. A system in computer readable form containing modules with defined amino acid characteristics wherein each module having an assigned value or weight for one or more profiles.
